(19) United States
(12) Patent Application Publication
(10) Pub. No.: US 2020/0127210 A1
(43) Pub. Date: Apr. 23, 2020

(54) ORGANIC ELECTROLUMINESCENT ELEMENT MATERIAL, ORGANIC ELECTROLUMINESCENT ELEMENT, DISPLAY DEVICE, LIGHTING DEVICE, AND COMPOUND

(71) Applicant: Konica Minolta, Inc., Tokyo (JP)

(72) Inventors: Yukihiro MAKISHIMA, Hino-shi, Tokyo (JP); Noriko UEDA, Hachioji-shi, Tokyo (JP); Yasuo MIYATA, Yokohama-shi, Kanagawa (JP)

(21) Appl. No.: 16/477,742

(22) PCT Filed: Feb. 13, 2018
(86) PCT No.: PCT/JP2018/004784

§ 371 Date: Jul. 12, 2019

(30) Foreign Application Priority Data
Mar. 16, 2017 (JP) 2017-050723

(51) Int. Cl. H01L 51/00; C07D 403/10; C09K 11/06; C07D 491/147; C07D 487/14; C07D 405/14; C07D 491/048

(52) U.S. Cl. CPC H01L 51/0067; C07D 403/10; C09K 11/06; H01L 51/0072; C07D 491/147; H01L 51/5016; C07D 405/14; H01L 51/0073; C07D 491/048; C09K 2211/1018; C07D 487/14

(57) ABSTRACT

An organic electroluminescent element material containing a compound having a structure represented by Formula (1):

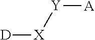

wherein D and A each represent a substituent; X and Y each represent a carbon atom, a nitrogen atom, an oxygen atom or a silicon atom which may have a hydrogen atom or a substituent; and at least one of X and Y is a carbon atom, provided that in the substituent represented by D, when a structure in which a linking portion to a linker (X—Y) is replaced with a hydrogen atom is D-H; and in the substituent represented by A, when a structure in which a linking portion to the linker (X—Y) is replaced with a hydrogen atom is A-H, D-H has a higher energy level of HOMO than A-H, and A-H has a lower energy level of LUMO than D-H.

### TECHNICAL FIELD

The present invention relates to an organic electroluminescent element material and compound having enhanced luminous efficiency, an organic electroluminescent element using this material, a display device and a lighting device equipped with the organic electroluminescent element.

### BACKGROUND

Organic electroluminescent elements (hereinafter, also referred to as “organic EL elements”), which are based on electroluminescence of organic materials, have already been put into practice as a new generation of light emitting systems capable of achieving planar light emission. Organic EL elements have recently been applied to electronic displays and also to lighting devices and display devices. Thus, further development of organic EL elements is expected.

As a light emission mode of an organic EL, there are two types. One is “a phosphorescence emission type” which emits light when a triplet excited state returns to a ground state, and another one is “a fluorescence emission type” which emits light when a singlet excited state returns to a ground state.

When an electric filed is applied to an organic EL element, a hole and an electron are respectively injected from an anode and a cathode, they are recombined in a light emitting layer to produce an exciton. At this moment, a singlet exciton and a triplet exciton are formed with a ratio of 25%:75%. Therefore, it is known that a phosphorescence emission type using a triplet exciton will produce theoretically high internal quantum efficiency compared with a fluorescence emission type.

However, in order to obtain high quantum efficiency in a phosphorescence emission type, it is required to use a complex compound having a rare metal of iridium or platinum in the center metal. This may induce industrial problems such as depletion of reserves of rare metals and increase in price of metals in the future.

On the other hand, in recent years, new techniques relevant to a fluorescence emission type have been proposed to improve luminous efficiency.

For example, Patent Document 1 discloses a technique which is focused on a phenomenon wherein singlet excitons are generated by collision of two triplet excitons (it is called as Triplet-Triplet Annihilation (TTA), or Triplet-Triplet Fusion (TTF)), and which improves luminous efficiency of a fluorescent element by allowing the TTA phenomenon to occur effectively. Although this technique may increase power efficiency of a fluorescence emission material (hereafter, it is called as a fluorescent emission material or fluorescent material) from two to three times larger than the power efficiency of a conventional fluorescent material, the luminous efficiency in TTA is not as high as that of the aforementioned phosphorescent material due to a theoretical limitation, because the rate of conversion of the excited triplet energy level to the excited singlet energy level will remain to about 40%.

Recent studies have disclosed a fluorescent material that employs a thermally activated delayed fluorescent mechanism (hereinafter also referred to as “TADF”) that causes reverse intersystem crossing (“RISC”) from a triplet exciton to a singlet exciton. It is reported that it may be applied to an organic EL element (for example, refer to Patent Document 2 and Non-patent Documents 1 to 2). By making use of this delayed fluorescence caused by the TADF mechanism, theoretically, it is possible to achieve an internal quantum efficiency of 100% in fluorescence emission, which is similar to the phosphorescent emission, in fluorescent emission by electric field excitation.

In order to make appear the TADF phenomenon, it is required that a reverse intersystem crossing (RISC) from the triplet state, which is produced with an amount of 75% by an electric field excitation, to the singlet state should be taken place at room temperature or at a light emitting layer temperature in the organic EL element. Further, by the mechanism that the singlet exciton produced by the reverse intersystem crossing emits fluorescence in the same way as the singlet exciton directly produced with an amount of 25%, it is theoretically possible to realize 100% internal quantum efficiency. In order to make appear this reverse intersystem crossing, it is necessary that the absolute value of the difference between the singlet excited level (S₁) and the triplet excited level (T₁) (hereafter, it is called as ΔE_{ST}) is very small.

Further, it is known that a method in which a compound having a TADF property is contained as a third component (assist-dopant) in a light emitting layer mainly containing a host compound and a light emitting compound is effective for exhibiting high luminous efficiency (for example, refer to None-patent Document 3). By producing 25% singlet excitons and 75% triplet excitons by electric field excitation on the assist-dopant, the triplet excitons may produce singlet excitons accompanied with reverse intersystem crossing (RISC). The energy of singlet excitons is transferred to the light emitting compound, and the light emitting compound may emits light by the energy transferred thereto. Therefore, it is possible to cause the light emitting compound to emit light using 100% of the exciton energy theoretically, and high luminous efficiency is expressed.

In order to express the TADF phenomenon, it is necessary to localize the highest occupied molecular orbital (HOMO) and the lowest unoccupied molecular orbital (LUMO) in the molecule without mixing them, and to minimize ΔE_{ST}.

Next, an example of molecular design for causing the TADF phenomenon to appear is described.

FIG. 1A, FIG. 1B, FIG. 2A and FIG. 2B are the schematic diagrams indicating the energy diagram of the compound (TADF compound) which expresses the TADF phenomenon, and a general fluorescent compound. For example, in the case of 2CzPN (4,5-bis(cathazol-9-yl)-1,2-dicyanobenzene) illustrated in FIG. 1A, HOMO is localized at carbazolyl groups at the 1 position and the 2 position of the benzene ring, and LUMO is localized at cyano groups at the 4 position and the 5 position. As a result, as indicated in FIG. 1B, the HOMO and the LUMO of 2CzPN may be separated, and ΔE_{ST} becomes very small. Thus a TADF phenomenon will be produced. On the other hand, in the case of 2CzXy (refer to FIG. 2A and FIG. 2B) which is produced by substituting cyano groups at the 4 position and the 5 position of 2CzPN with methyl groups, the HOMO and the LUMO are not clearly separated as is seen in 2CzPN. As a result, ΔE_{ST} may not be made small, and a TADF phenomenon will not be produced.

In order to separate HOMO and LUMO, as described above, there are methods such as twisting the junction of the HOMO and the LUMO with a strong electron donating group and electron withdrawing group, and increasing the distance between the HOMO and the LUMO. However, these methods have large molecular design limitations. Therefore, when designing a high-performance organic EL element, there are problems in performing molecular design with desired physical properties, such as HOMO level, LUMO level, light emission wavelength, and quantum yield in the TADF material.

In addition to TADF, exciplex luminescence is known as a technology for minimizing ΔE_{ST} and increasing luminous efficiency (for example, refer to Non-patent Document 4). According to this method, an exciplex can be formed in a thin film by co-evaporating an electron donating molecule and an electron withdrawing molecule. In the exciplex state, it is known that ΔE_{ST} is minimal, and it is possible to make emit EL light using 100% of the theoretical exciton energy, in the same manner as TADF. In order to form an exciplex between molecules, it is necessary to use strong electron donating molecules and electron withdrawing molecules. However, when such a configuration is adopted, an excited state having a strong intramolecular charge transfer (CT) property is formed, which causes the emission spectrum to be greatly elongated. Therefore, there is a problem that it is difficult to control the emission wavelength. Conversely, weakening the electron donating or electron withdrawing properties of each molecule in order to control the emission wavelength would interfere with exciplex emission. Therefore, development of a new method for forming an exciplex that achieves high luminous efficiency while controlling emission wavelength is desired.

### PRIOR ART DOCUMENTS

### Patent Documents

Patent Document 1: WO 2010/134350

Patent Document 2: JP-A 2013-116975

### Non-Patent Documents

Non-patent document 1: H. Uoyama, et al., Nature, 2012, 492, 234-238.

Non-patent document 2: Q. Zhang, et al., Nature, Photonics, 2014, 8, 326-332.

Non-patent document 3: H. Nakanotani, et al., Nature Communication, 2014, 5, 4016-4022.

Non-patent Document 4: K. Goushi, et al., Appl. Phys. Lett. 2012, 101, 023306.

### SUMMARY OF THE INVENTION

### Problems to be Solved by the Invention

The present invention has been made in view of the above problems and circumstances. An object of the present invention is to provide an organic electroluminescent element material and compound capable of forming a new exciplex enabling to enhance luminous efficiency. Another object of the present invention is to provide an organic electroluminescent element containing the organic electroluminescent element material, and a display device and a lighting device equipped with the organic electroluminescent element.

### Means to Solve the Problems

The present inventors have investigated the cause of the above-described problems in order to solve the problems. As a result, it was found to enhance luminous efficiency which is an aimed effect of the present invention by using an organic electroluminescent element material having a structure represented by Formula (1) capable of forming an intramolecular or intermolecular exciplex with one type of molecule.

That is, the above-described problems of the present invention are solved by the following embodiments.
1. An organic electroluminescent element material comprising a compound having a structure represented by Formula (1).

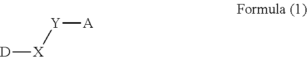

In the formula, D and A each represent a substituent; X and Y each represent a carbon atom, a nitrogen atom, an oxygen atom or a silicon atom which may have a hydrogen atom or a substituent; and at least one of X and Y is a carbon atom.

In the substituent represented by D, when a structure in which a linking portion to a linker (X—Y) is replaced with a hydrogen atom is D-H; and in the substituent represented by A, when a structure in which a linking portion to the linker (X—Y) is replaced with a hydrogen atom is A-H, D-H has a higher energy level of a highest occupied molecular orbital (HOMO) than A-H, and A-H has a lower energy level of a lowest unoccupied molecular orbital (LUMO) than D-H.

The substituent represented by D has a number of ring structures in the range of 3 to 15, and each of the ring structures may be bonded or condensed to each other.

The structure represented by Formula (1) may further have one or more substituents, and a plurality of the substituents may be bonded to each other to form a ring structure. In addition, one saturated ring containing X and Y as ring member atoms may be formed.
2. The organic electroluminescent element material described in the embodiment 1, wherein the ring structure represented by D in Formula (1) is a 5- or 6-membered aromatic hydrocarbon ring or heteroaromatic ring, and the compound contains three or more aforesaid ring structures.
3. The organic electroluminescent element material described in the embodiment 1 or 2, wherein the substituent represented by A in Formula (1) has a ring structure, the ring structure is a 5- or 6-membered aromatic hydrocarbon ring or heteroaromatic ring, and the compound contains at least one ring aforesaid structure.
4. The organic electroluminescent element material described in any one of the embodiments 1 to 3, wherein the substituent represented by D in Formula (1) has one selected from the group consisting of a carbazole ring, an indolocarbazole ring, a diindolocarbazole ring, an acridan ring, and an indoloindole ring.
5. The organic electroluminescent element material described in any one of the embodiments 1 to 4, wherein the substituent represented by A in Formula (1) is at least one selected from the group consisting of a pyridine ring, a pyrimidine ring, a triazine ring, a dibenzofuran ring, an azadibenzofuran ring, a diazadibenzofuran ring, a carboline ring, a diazacarbazole ring, and a benzene ring containing at least one selected from a cyano group, a trifluoromethyl group and a halogen atom.
6. The organic electroluminescent element material described in any one of the embodiments 1 to 5, wherein the substituent represented by A in Formula (1) has two or more hetero atoms.
7. The organic electroluminescent element material described in any one of the embodiments 1 to 6, wherein X and Y in Formula (1) forms an ethylene linker.
8. The organic electroluminescent element material described in any one of the embodiments 1 to 6, wherein in Formula (1), the ring formed by bonding the substituents on X and Y to each other is a cyclohexyl ring, and the substituent represented by D and the substituent represented by A are each bonded to the cyclohexyl ring by syn addition.
9. The organic electroluminescent element material described in any one of the embodiments 1 to 8, wherein the electroluminescent element material is a light emitting material.
10. The organic electroluminescent element material described in any one of the embodiments 1 to 8, wherein the electroluminescent element material is a charge transport material.
11. The organic electroluminescent element material described in any one of the embodiments 1 to 10, wherein the compound having a structure represented by Formula (1) is a compound that forms an intramolecular or intermolecular exciplex.
12. An organic electroluminescent element having an anode, a cathode, and a light emitting layer between the anode and the cathode, wherein at least one of the light emitting layers contains the electroluminescent element material described in any one of the embodiments 1 to 11.
13. The organic electroluminescent element described in the embodiment 12, wherein the light emitting layer further contains a host compound.
14. The organic electroluminescent element described in the embodiment 12 or 13, wherein the light emitting layer further contains at least one of a fluorescence emitting compound and a phosphorescence emitting compound.
15. The organic electroluminescent element described in any one of the embodiments 12 to 14, wherein the light emitting layer further contains a host compound and at least one of a fluorescence emitting compound and a phosphorescence emitting compound.
16. A display device provided with the organic electroluminescent element described in any one of the embodiments 12 to 15.
17. A lighting device provided with the organic electroluminescent element described in any one of the embodiments 12 to 15.
18. A compound having a structure represented by Formula (1).

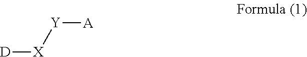

In the formula, D and A each represent a substituent; X and Y each represent a carbon atom, a nitrogen atom, an oxygen atom or a silicon atom which may have a hydrogen atom or a substituent; and at least one of X and Y is a carbon atom.

In the substituent represented by D, when a structure in which a linking portion to a linker (X—Y) is replaced with a hydrogen atom is D-H; and in the substituent represented by A, when a structure in which a linking portion to the linker (X—Y) is replaced with a hydrogen atom is A-H, D-H has a higher energy level of a highest occupied molecular orbital (HOMO) than A-H, and A-H has a lower energy level of a lowest unoccupied molecular orbital (LUMO) than D-H.

The substituent represented by D has a number of ring structures in the range of 3 to 15, and each of the ring structures may be bonded or condensed to each other.

The structure represented by Formula (1) may further have one or more substituents, and a plurality of the substituents may be bonded to each other to form a ring structure. In addition, one saturated ring containing X and Y as ring member atoms may be formed.
19. The compound described in the embodiment 18, wherein the ring structure resented by D in Formula (1) is a 5- or 6-membered aromatic hydrocarbon ring or heteroaromatic ring, and the compound contains three or more substituents represented by D.
20. The compound described in the embodiment 18 or 19, wherein the substituent represented by A in Formula (1) is a 5- or 6-membered aromatic hydrocarbon ring or heteroaromatic ring, and the compound contains at least one substituent represented by A.
21. The compound described in any one of the embodiments 18 or 20, wherein the substituent represented by D in Formula (1) has one selected from the group consisting of a carbazole ring, an indolocarbazole ring, a diindolocarbazole ring, an acridan ring, and an indoloindole ring.
22. The compound described in any one of the embodiments 18 to 21, wherein the substituent represented by A in Formula (1) is at least one selected from the group consisting of a pyridine ring, a pyrimidine ring, a triazine ring, a dibenzofuran ring, an azadibenzofuran ring, a diazadibenzofuran ring, a carboline ring, a diazacathazole ring, and a benzene ring containing at least one selected from a cyano group, a trifluoromethyl group and a halogen atom.
23. The compound described in any one of the embodiments 18 to 22, wherein the substituent represented by A in Formula (1) contains two or more hetero atoms.
24. The compound described in any one of the embodiments 18 to 23, wherein X and Y in Formula (1) forms an ethylene linker.
25. The compound described in any one of the embodiments 18 to 23, wherein in Formula (1), the ring formed by bonding the substituents on X and Y to each other is a cyclohexyl ring, and the substituent represented by D and the substituent represented by A are each bonded to the cyclohexyl ring by syn addition.

### Effects of the Invention

By the above-described embodiments of the present invention, it is possible to provide an organic electroluminescent material and compound capable of enhancing luminous efficiency. It is also possible to provide an organic electroluminescent element to which the organic electroluminescent material is applied, and a display device and a lighting device equipped with the organic electroluminescent element.

First, the intermolecular exciplex and the intramolecular exciplex in the present invention will be described.

In general, an exciplex (also referred to as an excited complex) refers to a species of an excited complex ABₙ* that is formed by a species A* in the excited electronic state with n species B in the ground state. In particular, the case where A and B are the same species and forms a 1:1 complex is called an excimer.

In the present invention, an excited complex including an excimer as described above is called an exciplex. An excited complex formed between the partial structures in the molecule is called an intramolecular exciplex, and an excited complex formed between identical species is called an intermolecular exciplex.

### <Intermolecular Exciplex>

The following compound is a representative compound of the present invention (exemplified compound E-22) which expresses an intermolecular exciplex.

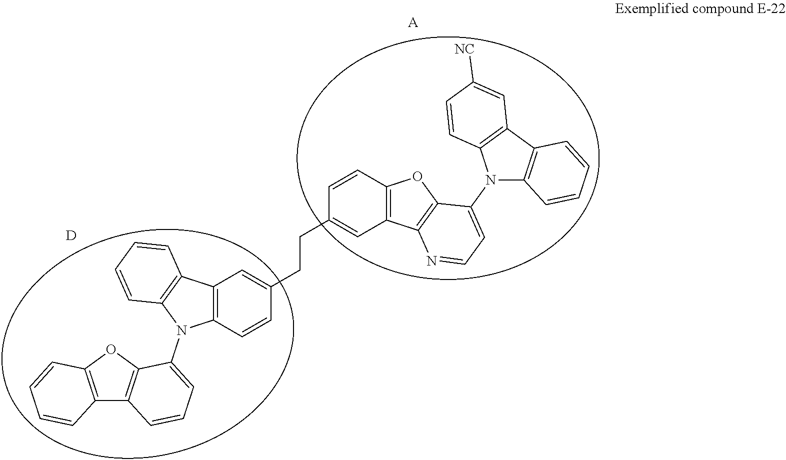

In the above-mentioned exemplified compound E-22, the substituent enclosed by the frame indicated by D corresponds to the substituent represented by D in Formula (1), and the substituent enclosed by the frame indicated by A corresponds to the substituent represented by A in Formula (1).

The above-described exemplified compound E-22 exerts an exciplex between two molecules to cause an effect as shown below, and this is referred to as an intermolecular exciplex.

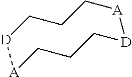

### <Intramolecular Exciplex>

The following compound is a representative compound of the present invention (exemplified compound E-77) which expresses an intramolecular exciplex.

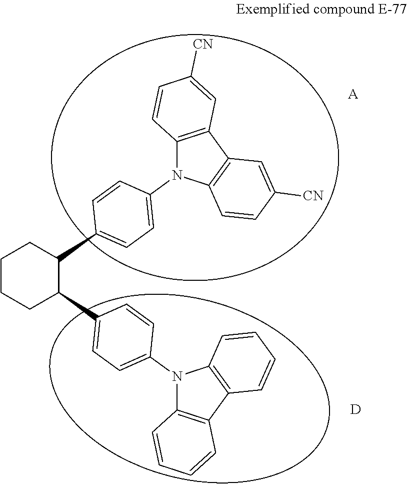

In the above exemplified compound E-77, the substituent enclosed by the frame indicated by D corresponds to the substituent represented by D in Formula (1), and the substituent enclosed by the frame indicated by A corresponds to the substituent represented by A in Formula (1). Further, X and Y in Formula (1) are constituent atoms (carbon atoms) of a cyclohexane ring.

The above-mentioned exemplified compound E-77 exerts an effect by forming an exciplex in one molecule, and this is called an intramolecular exciplex.

The mechanism for expressing the effects of the present invention and the mechanism of action are not all clear, but are presumed as follows.

In contrast to an organic EL element containing a compound that forms a conventional intermolecular exciplex between two kinds of molecules, an organic EL element containing the compound which has a structure represented by Formula (1) of the present invention can reduce the number of materials in a light emitting layer. As a result, it is possible to enhance the functionality of the organic EL element (increase the luminous efficiency) by forming a uniform film, and to reduce the process cost in the deposition process and the application process. In a thin film, since an exciplex is formed by the interaction of two sites, the effect may be maximized when the mixing ratio is 1:1. However, in forming a conventional mixed film of two kinds of molecules, it is difficult to perform film formation with a mixing ratio of 1:1 precisely because of process limitations. On the other hand, by using a compound having a structure represented by Formula (1) of the present invention, for example, a substituent represented by D (hereinafter, also referred to as “substituent D” or “electron donating group D”) a substituent represented by A (hereinafter “substituent A” or “the electron withdrawing group A” that form an exciplex may be completely controlled to 1:1. Therefore, the effect of the exciplex may be maximized, and luminous efficiency of the organic EL element may be enhanced

Further, an organic EL element containing the compound forming the intermolecular or intramolecular exciplex according to the present invention is capable of effectively suppress lengthening the emission wavelength more than the organic EL element containing the compound forming the conventional intermolecular exciplex.

This is because the intramolecular or intermolecular exciplex according to the present invention is easily formed at a close intramolecular distance, as described above, a strong electron donating group D and a strong electron withdrawing group A are not required, unlike an organic EL element containing a compound that forms an intermolecular exciplex from two types of molecules, which is a prior art. As a result, an organic EL element containing the compound forming the intramolecular or intermolecular exciplex of the present invention is capable of suppressing a longer emission wavelength than the organic EL element containing the compound forming the conventional intermolecular exciplex.

In the present invention, the substituent represented by D (electron donating group D) and the substituent represented by A (electron withdrawing group A) are linked by —X—Y—, for example, an ethylene group or a cyclohexyl group as a linker. By using this new method, it is possible to form an intramolecular or intermolecular exciplex and minimize ΔE_{ST}. Furthermore, the intramolecular or intermolecular exciplex according to the present invention is easily formed because the substituent D which is an electron donating group and the substituent A which is an electron withdrawing group may be present at a short distance in the molecule. As a result of these, it is presumed that high luminescence and high luminous efficiency can be achieved compared to the compounds forming the intermolecular exciplex of the prior art.

The structure of the substituent D which is an electron donating group and the substituent A which is an electron withdrawing group are usually too small in molecular weight even if it is liquid or solid when each group is a single molecule. There is a problem that it becomes difficult to form a thin film using a vapor deposition method, and the vapor deposition method is hardly applied to the formation of an organic electroluminescent element. In the present invention, the molecular weight is increased by connecting the substituent D which is an electron donating group and the substituent A which is an electron withdrawing group, which have hitherto been impossible to produce an organic EL element, thus, application of the thin film formation method was made possible. Thereby it is possible to realize the production of a new organic EL element.

### BRIEF DESCRIPTION OF THE DRAWINGS

FIG. 1A is a drawing illustrating a structure of 2CzPN as an example of the TADF compound in which HOMO and LUMO are separated.

FIG. 1B is a schematic drawing illustrating an example of the energy diagram of the TADF compound illustrated in FIG. 1A.

FIG. 2A is a drawing illustrating a structure of 2CzXy which is an example of a general fluorescence emitting compound in which HOMO and LUMO are not clearly separated.

FIG. 2B is a schematic drawing illustrating an example of an energy diagram of a general fluorescence emitting compound illustrated in FIG. 2A.

FIG. 3 is a schematic drawing illustrating an example of an energy diagram when a compound having a structure represented by Formula (1) functions as an assist-dopant.

FIG. 4 is a schematic drawing illustrating an example of the energy diagram when a compound having a structure represented by Formula (1) functions as a host compound.

FIG. 5 is a schematic drawing for explaining HOMO and LUMO of a substituent D and a substituent A in Formula (1).

FIG. 6 is a schematic perspective view illustrating an example of a display device equipped with an organic EL element.

FIG. 7 is a schematic perspective view illustrating an example of the configuration of a display device by an active matrix method.

FIG. 8 is a schematic wiring diagram illustrating an example of a circuit of a light emitting pixel.

FIG. 9 is a schematic perspective view illustrating an example of a configuration of a display device by a passive matrix method.

FIG. 10 is a schematic perspective view illustrating an example of a configuration of a lighting device.

FIG. 11 is a schematic cross-sectional drawing illustrating an example of a configuration of a lighting device.

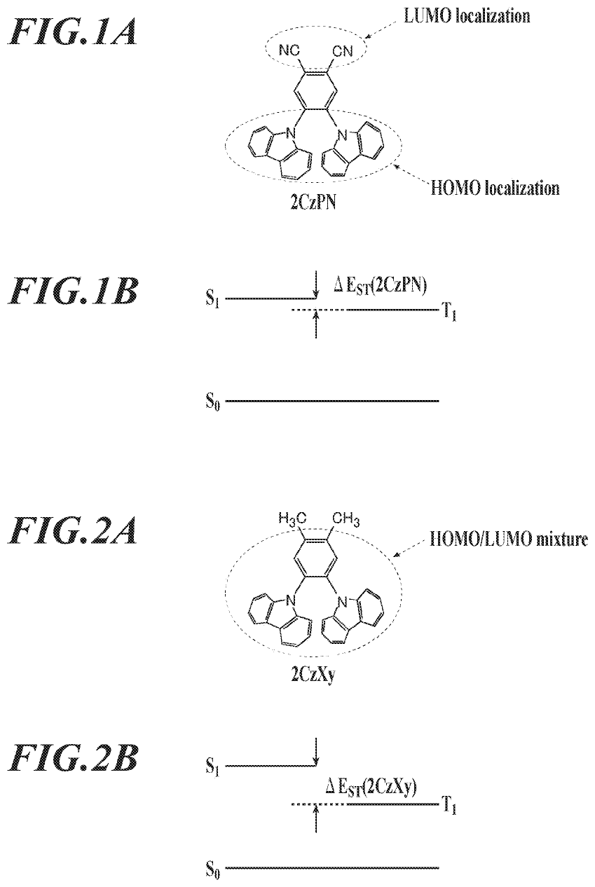

### EMBODIMENTS TO CARRY OUT THE INVENTION

The organic EL element material of the present invention contains a compound that forms an intramolecular or intermolecular exciplex with one type of molecule, that is, a compound having a structure represented by Formula (1) according to the present invention. This is a technical feature common to the inventions according to the respective claims

In the organic EL element material of the present invention, from the viewpoint of achieving the desired effects of the present invention, it is preferable that the ring structure represented by D in Formula (1) is a 5- or 6-membered aromatic hydrocarbon ring or heteroaromatic ring, and the compound contains three or more ring structures. It is also preferable that the substituent represented by A in Formula (1) has a ring structure, the ring structure is a 5- or 6-membered aromatic hydrocarbon ring or heteroaromatic ring, and the compound contains at least one ring structure. These are preferable from the viewpoint of obtaining an intramolecular or intermolecular exciplex with higher luminous efficiency.

It is preferable that the substituent represented by D (electron donating group D) in Formula (1) has one selected from the group consisting of a carbazole ring, an indolocarbazole ring, a diindolocathazole ring, an acridan ring, and an indoloindole ring from the viewpoint of obtaining an intramolecular or intermolecular exciplex with higher luminous efficiency.

It is preferable that the substituent represented by A (electron withdrawing group A) in Formula (1) is at least one selected from the group consisting of a pyridine ring, a pyrimidine ring, a triazine ring, a dibenzofuran ring, an azadibenzofuran ring, a diazadibenzofuran ring, a carboline ring, a diazacarbazole ring, and a benzene ring containing at least one selected from a cyano group, a trifluoromethyl group and a halogen atom from the viewpoint of obtaining an intramolecular or intermolecular exciplex with higher luminous efficiency.

It is preferable that the substituent represented by A (electron withdrawing group A) in Formula (1) has two or more hetero atoms from the viewpoint of obtaining an intramolecular or intermolecular exciplex with higher luminous efficiency.

It is preferable that X and Y in Formula (1) forms an ethylene linker from the viewpoint of obtaining an intramolecular or intermolecular exciplex with higher luminous efficiency.

It is preferable that the ring formed by bonding the substituents on X and Y to each other is a cyclohexyl ring, and the substituent represented by D and the substituent represented by A are each bonded to the cyclohexyl ring by syn addition from the viewpoint of obtaining an intramolecular or intermolecular exciplex with higher luminous efficiency.

In a preferred embodiment, the organic electroluminescent element material is a light emitting material or a charge transport material.

In addition, it is preferable that the organic electroluminescent element material is a compound which forms an intramolecular or intermolecular exciplex with one type of molecule from the viewpoint of achieving the intended effects of the present invention.

The present invention and the constitution elements thereof, as well as configurations and embodiments, will be detailed in the following. In the present description, when two figures are used to indicate a range of value before and after “to”, these figures are included in the range as a lowest limit value and an upper limit value.

### <<Organic Electroluminescent Element Material>>

An organic electroluminescent element material of the present invention is a compound having a structure represented by Formula (1).

In Formula (1), D and A each represent a substituent; X and Y each represent a carbon atom, a nitrogen atom, an oxygen atom or a silicon atom which may have a hydrogen atom or a substituent; and at least one of X and Y is a carbon atom.

### (HOMO and LUMO of Substituent D and Substituent A)

In a compound having a structure represented by Formula (1), the substituent D (electron donating group D) and the substituent A (electron withdrawing group A) each bonding to a linker group represented by X—Y satisfy the following relationship.

In the present invention, when a structure in which a linking portion to a linker (X—Y) is replaced with a hydrogen atom is D-H; and in the substituent represented by A, when a structure in which a linking portion to the linker (X—Y) is replaced with a hydrogen atom is A-H, D-H has a higher energy level of a highest occupied molecular orbital (HOMO) than A-H, and A-H has a lower energy level of a lowest unoccupied molecular orbital (LUMO) than D-H, as illustrated in FIG. 5.

When the respective LUMO and HOMO energy levels of D-H and A-H have the relationship as illustrated in FIG. 5, since the energy levels of the HOMO of D-H and the LUMO of A-H are close to each other, they are considered to be easy to form an exciplex.

That is, since the substituents D and A in the compound having a structure represented by Formula (1) may be in the same relationship as the above DH and AH, it is presumed that an intramolecular or an intermolecular exciplex is likely to be formed.

### (Substituents Represented by D)

The substituent represented by D in Formula (1) is not particularly limited as long as the above energy level relationship is satisfied. The substituent represented by D preferably has a number of ring structures in the range of 3 to 15, and each of the ring structures may be bonded or condensed to each other. Further, in a preferable embodiment, the ring structure represented by D in Formula (1) is a 5- or 6-membered aromatic hydrocarbon ring or heteroaromatic ring, and the compound contains three or more ring structures. The substituent represented by D preferably has one or two fused rings. In addition, D is preferably an electron donating group.

Examples of the substituent represented by D include: a diphenylamino group, a phenyl group substituted with a methoxy group, a pyrrole ring, an indole ring, a carbazole ring, an acridan ring, an indoloindole ring, a 9,10-dihydroacridine ring, a 10,11-dihydrodibenzazepine, a 5,10-dihydrodibenzoazacillin, a phenoxazine ring, a phenothiazine ring, a dibenzofuran ring, a dibenzothiophene ring, a benzofurylindole ring, a benzothienoindole ring, indolocarbazole ring, a diindolocarbazole ring, benzofurylcathazole ring, a benzothienocarbazole ring, a benzothienobenzothiophene ring, a benzocarbazole ring, and a dibenzocarbazole ring. Provided that two or more of the same or different substituents may be bonded to the above-described groups. In addition, the dibenzofuran ring functions as an electron donating group D as a whole when it is substituted by an electron donating substituent (for example, a carbazole group), however, when it is unsubstituted or substituted by an electron withdrawing substituent, it functions as an electron withdrawing group A (substituent A).

Further, in Formula (1), among the substituents D described above, it is preferable to have a carbazole ring, an indolocarbazole ring, a diindolocarbazole ring, an acridan ring, or an indoloindole ring.

### (Substituents Represented by A)

The substituent represented by A in Formula (1) is not particularly limited as long as the above energy level relationship is satisfied. The substituent represented by A preferably has a ring structure, and the ring structure is preferably a 5- or 6-membered aromatic hydrocarbon ring or heteroaromatic ring, and preferably has one or more of the ring structures. Moreover, it is a preferable embodiment that A is an electron withdrawing group.

Examples of the substituent represented by A include: a cyano group, a trifluoromethyl group, a halogen atom, a benzene ring which is substituted with a carbonyl group, a sulphonyl group, or a boryl group that may have a substituent, a dibenzofuran ring, an azadibenzofuran ring, a dibenzothiophene dioxide ring, a pyridine ring, a pyridazine ring, a pyrimidine ring, a pyrazine ring, a triazine ring, a quinoline ring, an isoquinoline ring, a quinazoline ring, a cinnoline ring, a quinoxaline ring, a phthalazine ring, a pteridin ring, a phenanthridine ring, a phenanthroline ring, an azacarbazole ring, a diazacarbazole ring, a dibenzofuran ring, a dibenzosilole ring, an azadibenzofuran ring, a diazadibenzofuran ring, a dibenzoborole ring, a dibenzophosphorous oxide ring, and a carboline ring. Provided that the same or different two or more of the above substituents may be bonded. It is also preferable that it is a structure which has a 2 or more hetero atoms among the said substituent.

In a preferable embodiment, the substituent represented by A is at least one selected from the group consisting of a pyridine ring, a pyrimidine ring, a triazine ring, a dibenzofuran ring, an azadibenzofuran ring, a diazadibenzofuran ring, a carboline ring, a diazacarbazole ring, and a benzene ring containing at least one selected from a cyano group, a trifluoromethyl group and a halogen atom.

### (X—Y Structure: Linker)

In Formula (1), X and Y each represent a carbon atom, a nitrogen atom, an oxygen atom or a silicon atom which may have a hydrogen atom or a substituent. Examples of a linker represented by —X—Y— include: —C—C—, —C—N—, —C—O—, and —C—Si—. More preferably, it is an ethylene linker (—CH₂—CH₂—).

In addition, one or two saturated rings containing X and Y as ring member atoms may be formed. Examples of the one or two saturated rings containing X and Y as ring member atoms are: a cyclopropane ring, a cyclobutane ring, a cyclopentane ring, a cyclohexane ring, a cycloheptane ring, a cyclooctane ring, a cyclononane ring, and a cyclodecane ring.

As the saturated ring, in particular, it is preferable that the ring formed by combining the substituents on X and Y with each other is a cyclohexyl ring, and it is preferable that the substituent represented by D and the substituent represented by A are each bonded to the cyclohexyl ring by syn addition.

Preferred specific examples of the compound having a structure represented by Formula (1) according to the present invention are listed below. These compounds may further have a substituent, or structural isomers may be present, and the present invention is not limited to the compounds exemplified below.

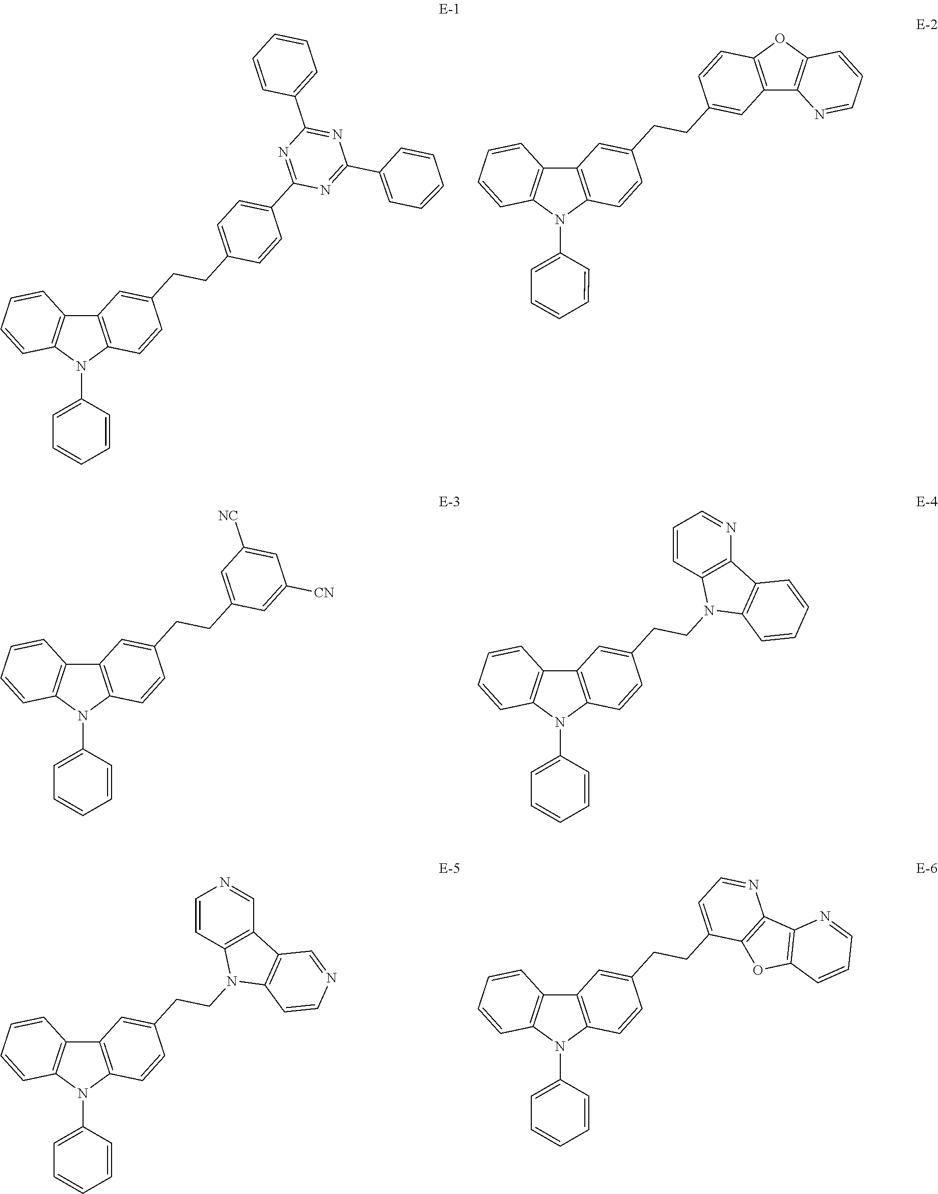

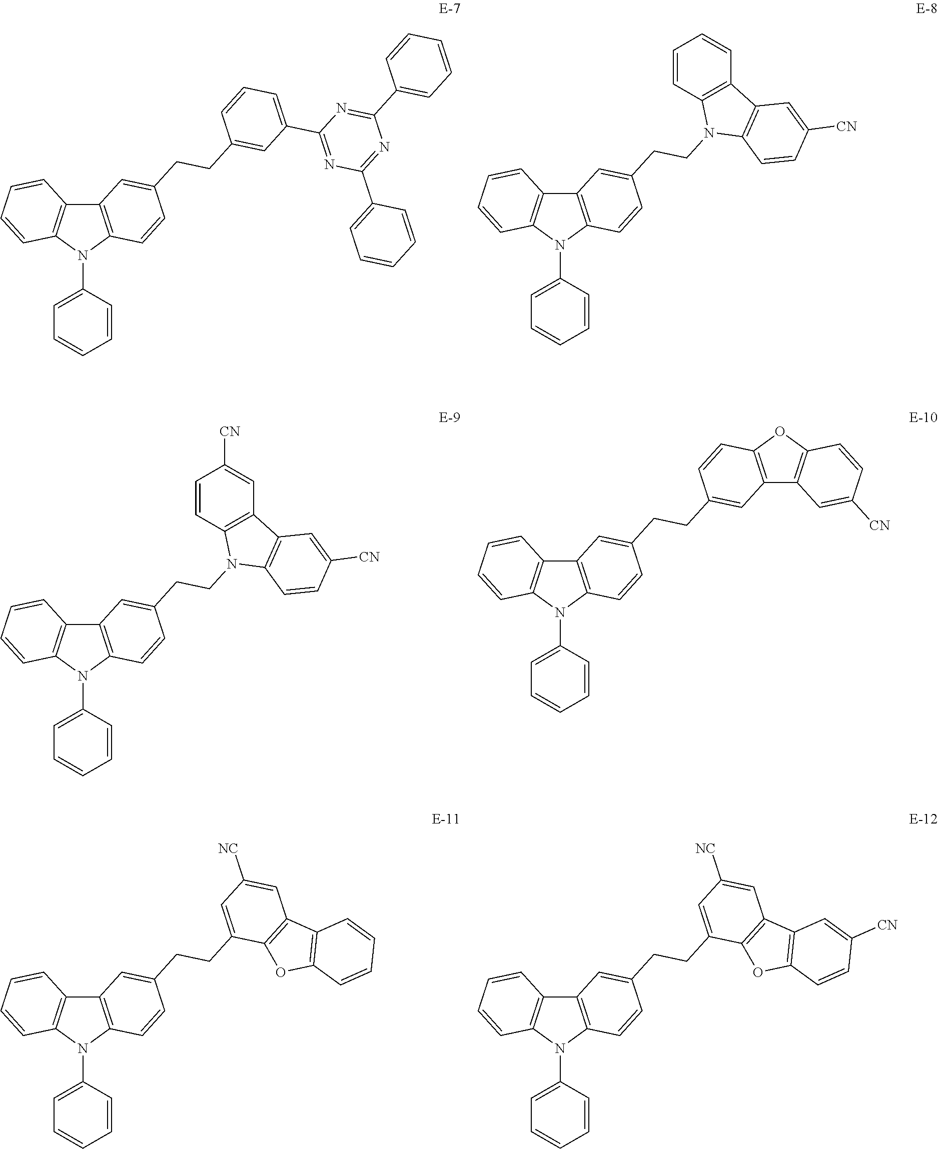 [SMILES: c1ccc(-c2nc(-c3ccccc3)nc(-c3cccc(CCc4ccc5c(c4)c4ccccc4n5-c4ccccc4)c3)n2)cc1 ; N#Cc1ccc2c(c1)c1ccccc1n2CCc1ccc2c(c1)c1ccccc1n2-c1ccccc1 ; N#Cc1ccc2c(c1)c1cc(C#N)ccc1n2CCc1ccc2c(c1)c1ccccc1n2-c1ccccc1 ; N#Cc1ccc2oc3ccc(CCc4ccc5c(c4)c4ccccc4n5-c4ccccc4)cc3c2c1 ; N#Cc1cc(CCc2ccc3c(c2)c2ccccc2n3-c2ccccc2)c2oc3ccccc3c2c1 ; N#Cc1ccc2oc3c(CCc4ccc5c(c4)c4ccccc4n5-c4ccccc4)cc(C#N)cc3c2c1]

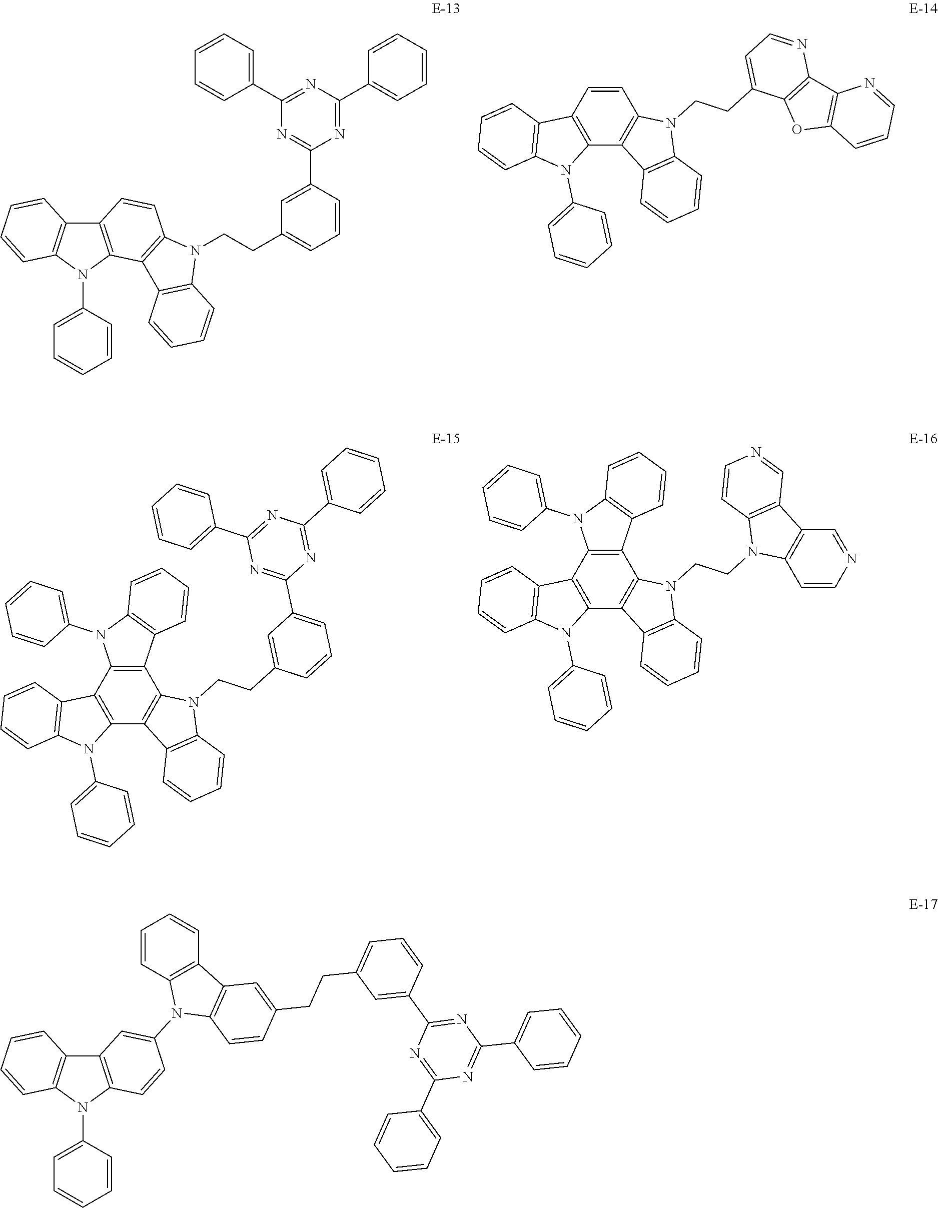 [SMILES: c1ccc(-c2nc(-c3ccccc3)nc(-c3cccc(CCn4c5ccccc5c5c4ccc4c6ccccc6n(-c6ccccc6)c45)c3)n2)cc1 ; c1ccc(-n2c3ccccc3c3ccc4c(c5ccccc5n4CCc4ccnc5c4oc4cccnc45)c32)cc1 ; c1ccc(-c2nc(-c3ccccc3)nc(-c3cccc(CCn4c5ccccc5c5c4c4c6ccccc6n(-c6ccccc6)c4c4c6ccccc6n(-c6ccccc6)c54)c3)n2)cc1 ; c1ccc(-n2c3ccccc3c3c4c(c5ccccc5n4CCn4c5ccncc5c5cnccc54)c4c(c5ccccc5n4-c4ccccc4)c32)cc1 ; c1ccc(-c2nc(-c3ccccc3)nc(-c3cccc(CCc4ccc5c(c4)c4ccccc4n5-c4ccc5c(c4)c4ccccc4n5-c4ccccc4)c3)n2)cc1]

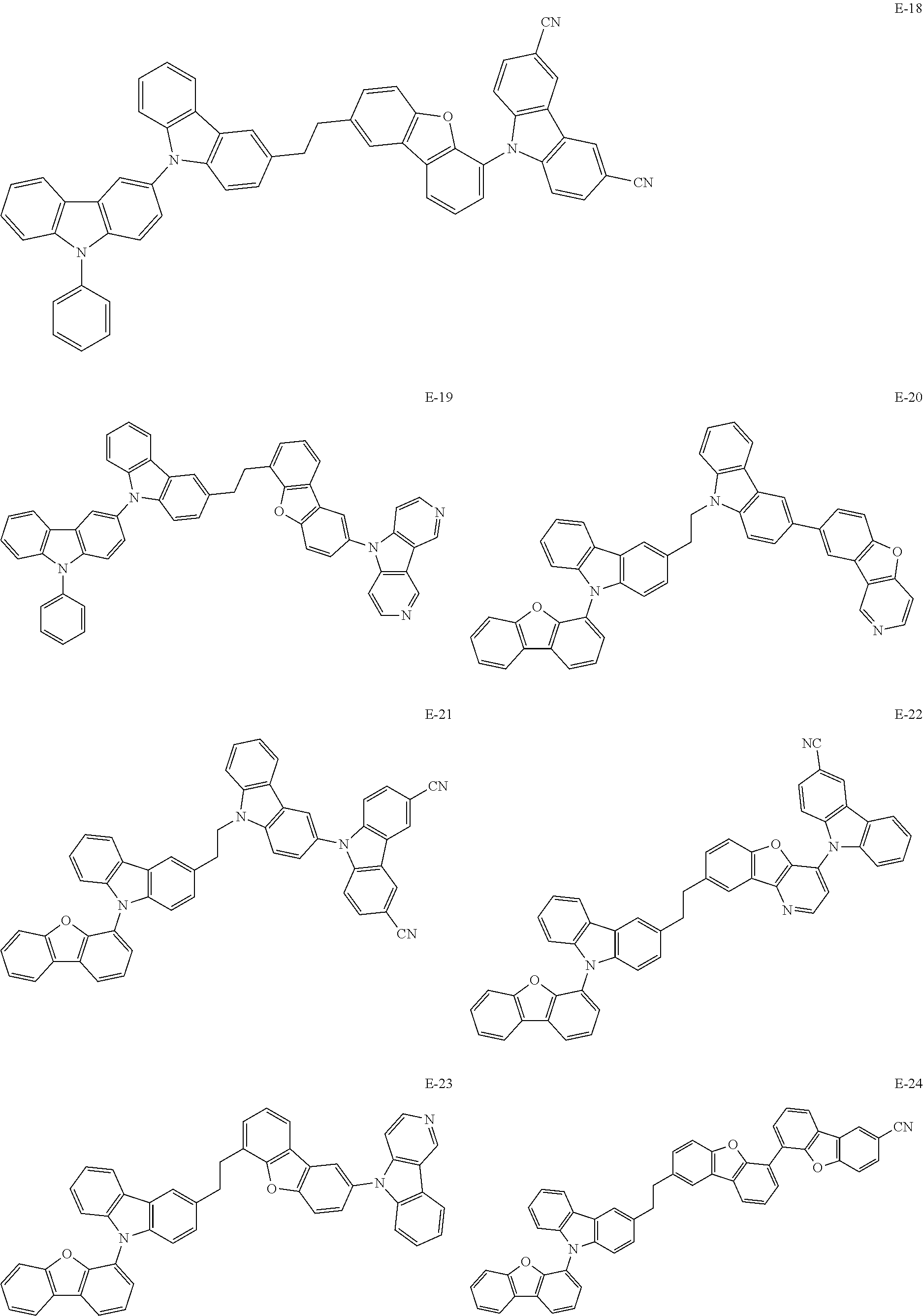

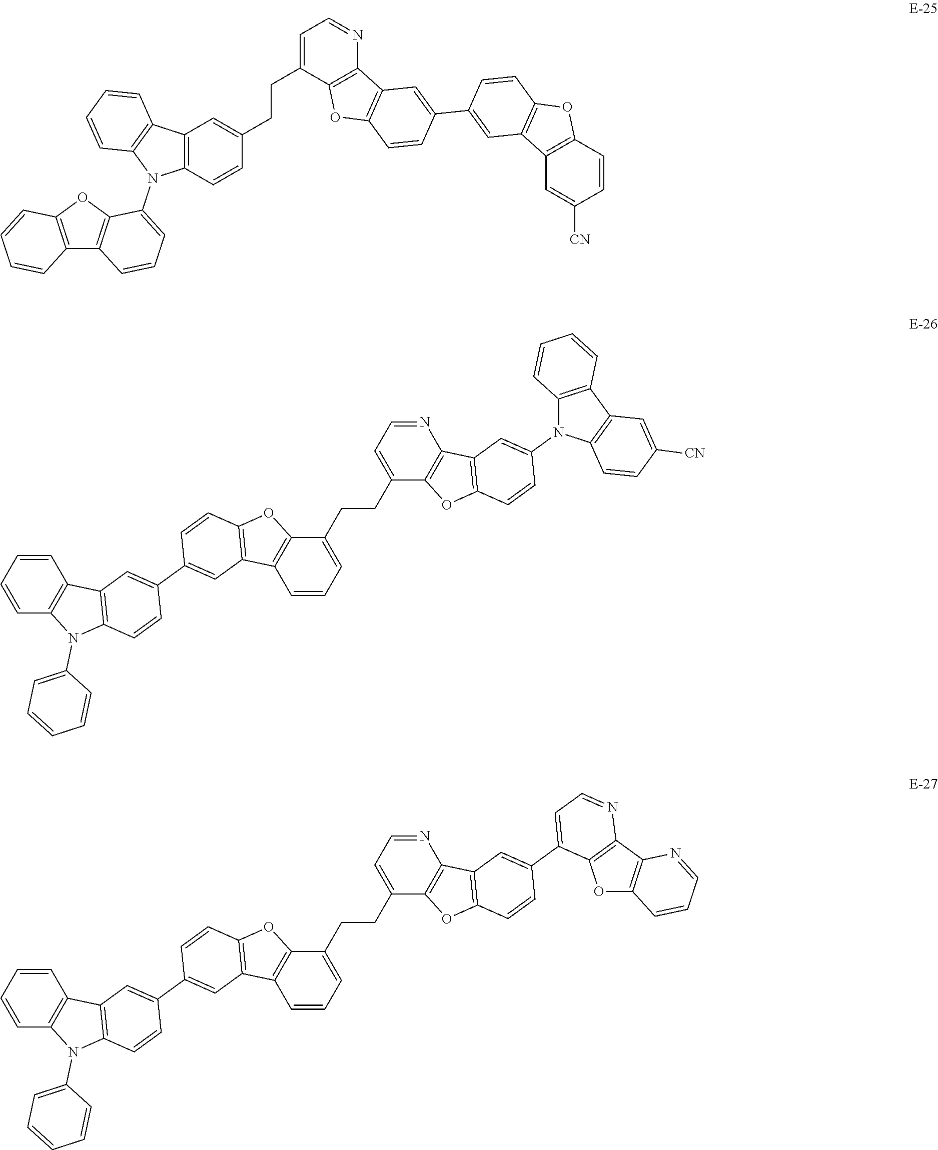 [SMILES: N#Cc1ccc2oc3ccc(-c4ccc5oc6c(CCc7ccc8c(c7)c7ccccc7n8-c7cccc8c7oc7ccccc78)ccnc6c5c4)cc3c2c1 ; N#Cc1ccc2c(c1)c1ccccc1n2-c1ccc2oc3c(CCc4cccc5c4oc4ccc(-c6ccc7c(c6)c6ccccc6n7-c6ccccc6)cc45)ccnc3c2c1 ; c1ccc(-n2c3ccccc3c3cc(-c4ccc5oc6c(CCc7ccnc8c7oc7ccc(-c9ccnc%10c9oc9cccnc9%10)cc78)cccc6c5c4)ccc32)cc1]

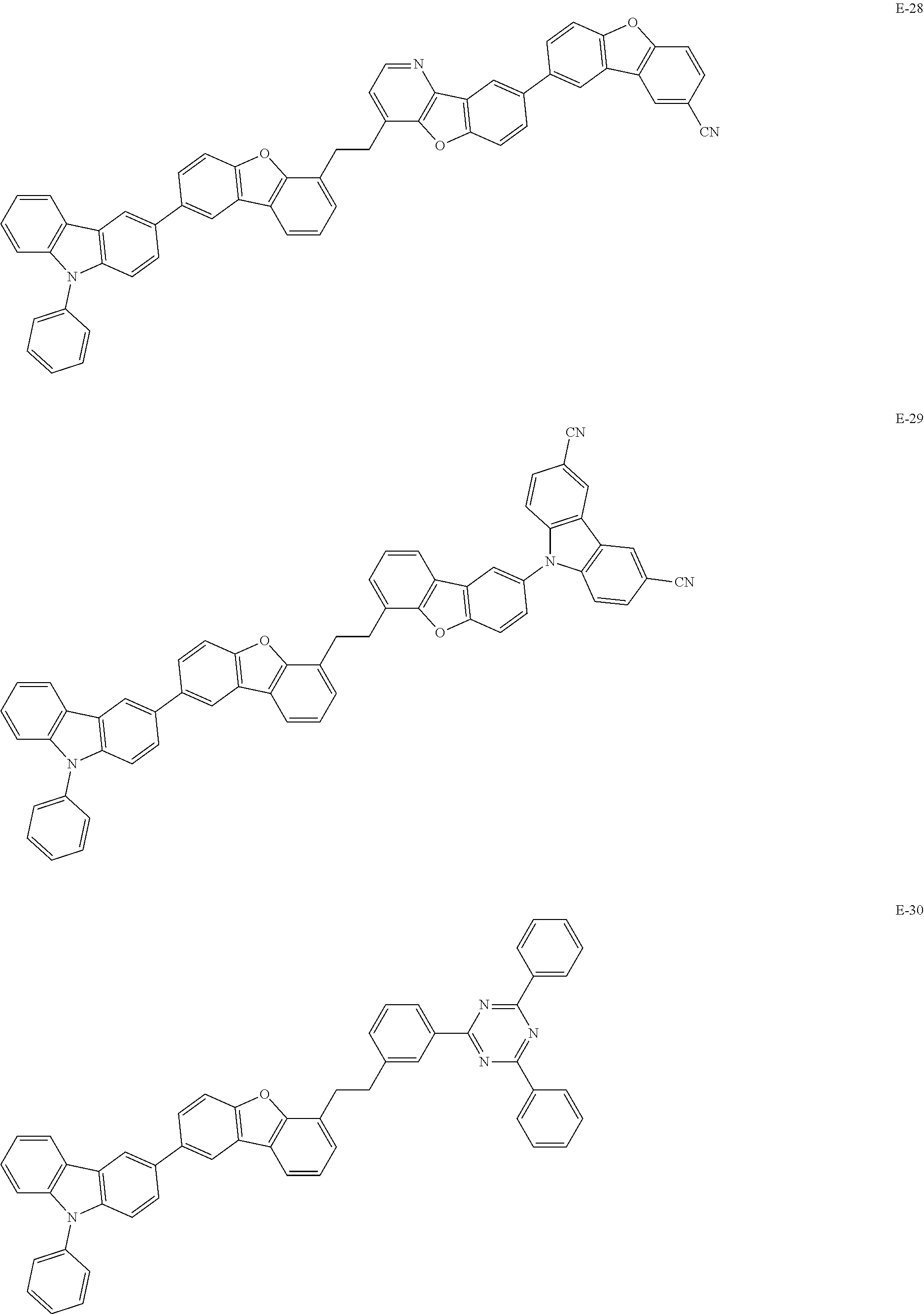 [SMILES: N#Cc1ccc2oc3ccc(-c4ccc5oc6c(CCc7cccc8c7oc7ccc(-c9ccc%10c(c9)c9ccccc9n%10-c9ccccc9)cc78)ccnc6c5c4)cc3c2c1 ; N#Cc1ccc2c(c1)c1cc(C#N)ccc1n2-c1ccc2oc3c(CCc4cccc5c4oc4ccc(-c6ccc7c(c6)c6ccccc6n7-c6ccccc6)cc45)cccc3c2c1 ; c1ccc(-c2nc(-c3ccccc3)nc(-c3cccc(CCc4cccc5c4oc4ccc(-c6ccc7c(c6)c6ccccc6n7-c6ccccc6)cc45)c3)n2)cc1]

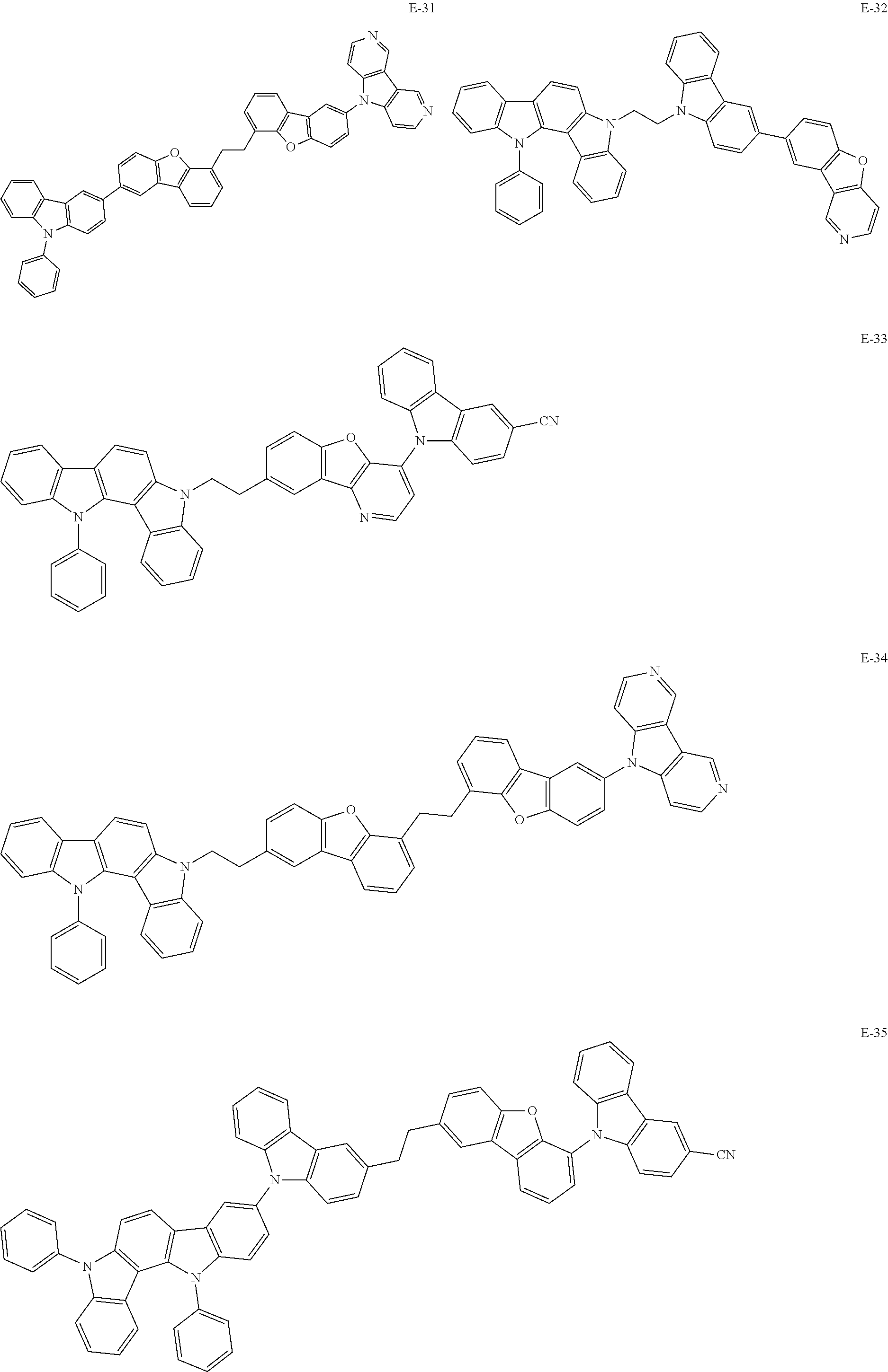

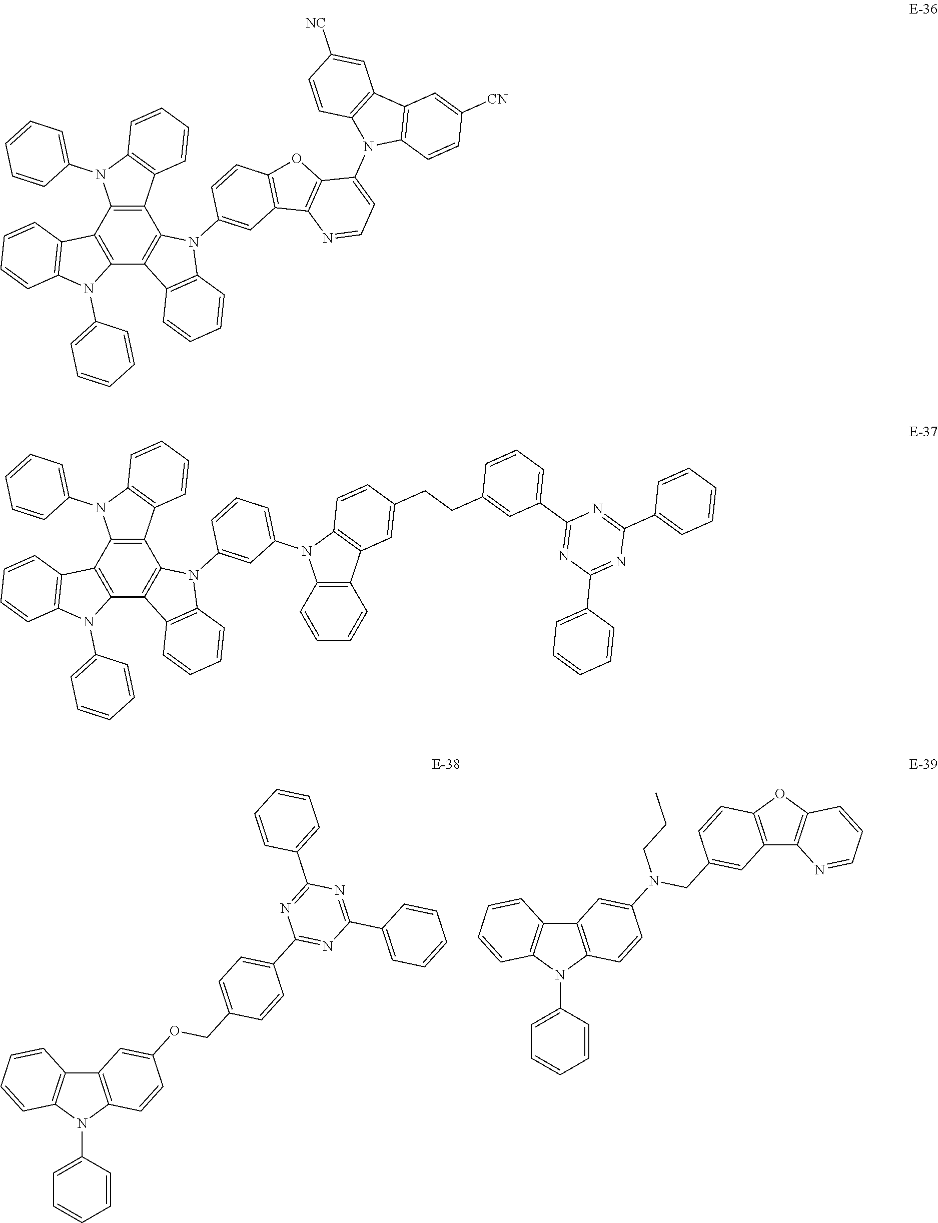

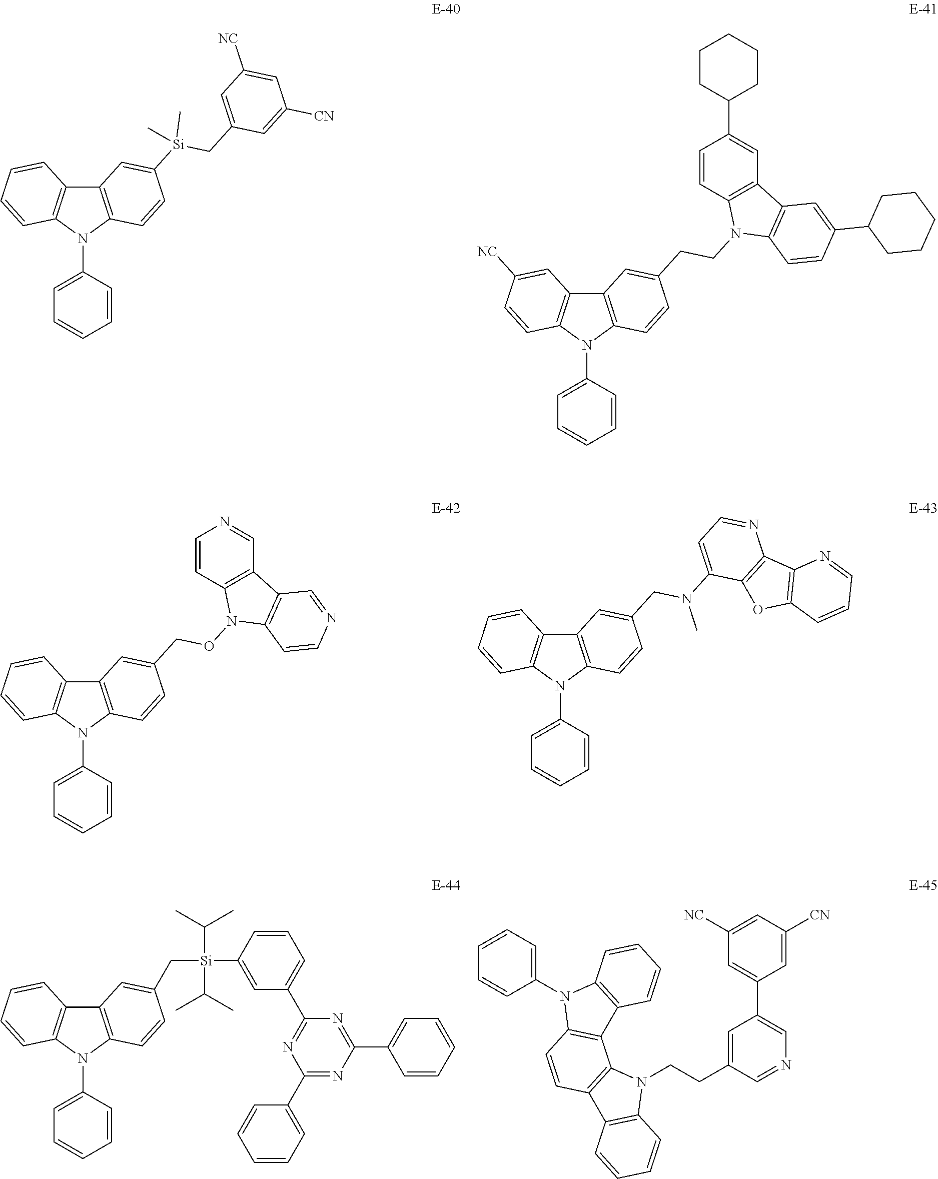 [SMILES: C[Si](C)(Cc1cc(C#N)cc(C#N)c1)c1ccc2c(c1)c1ccccc1n2-c1ccccc1 ; N#Cc1ccc2c(c1)c1cc(CCn3c4ccc(C5CCCCC5)cc4c4cc(C5CCCCC5)ccc43)ccc1n2-c1ccccc1 ; c1ccc(-n2c3ccccc3c3cc(COn4c5ccncc5c5cnccc54)ccc32)cc1 ; CN(Cc1ccc2c(c1)c1ccccc1n2-c1ccccc1)c1ccnc2c1oc1cccnc12 ; CC(C)[Si](Cc1ccc2c(c1)c1ccccc1n2-c1ccccc1)(c1cccc(-c2nc(-c3ccccc3)nc(-c3ccccc3)n2)c1)C(C)C ; N#Cc1cc(C#N)cc(-c2cncc(CCn3c4ccccc4c4ccc5c(c6ccccc6n5-c5ccccc5)c43)c2)c1]

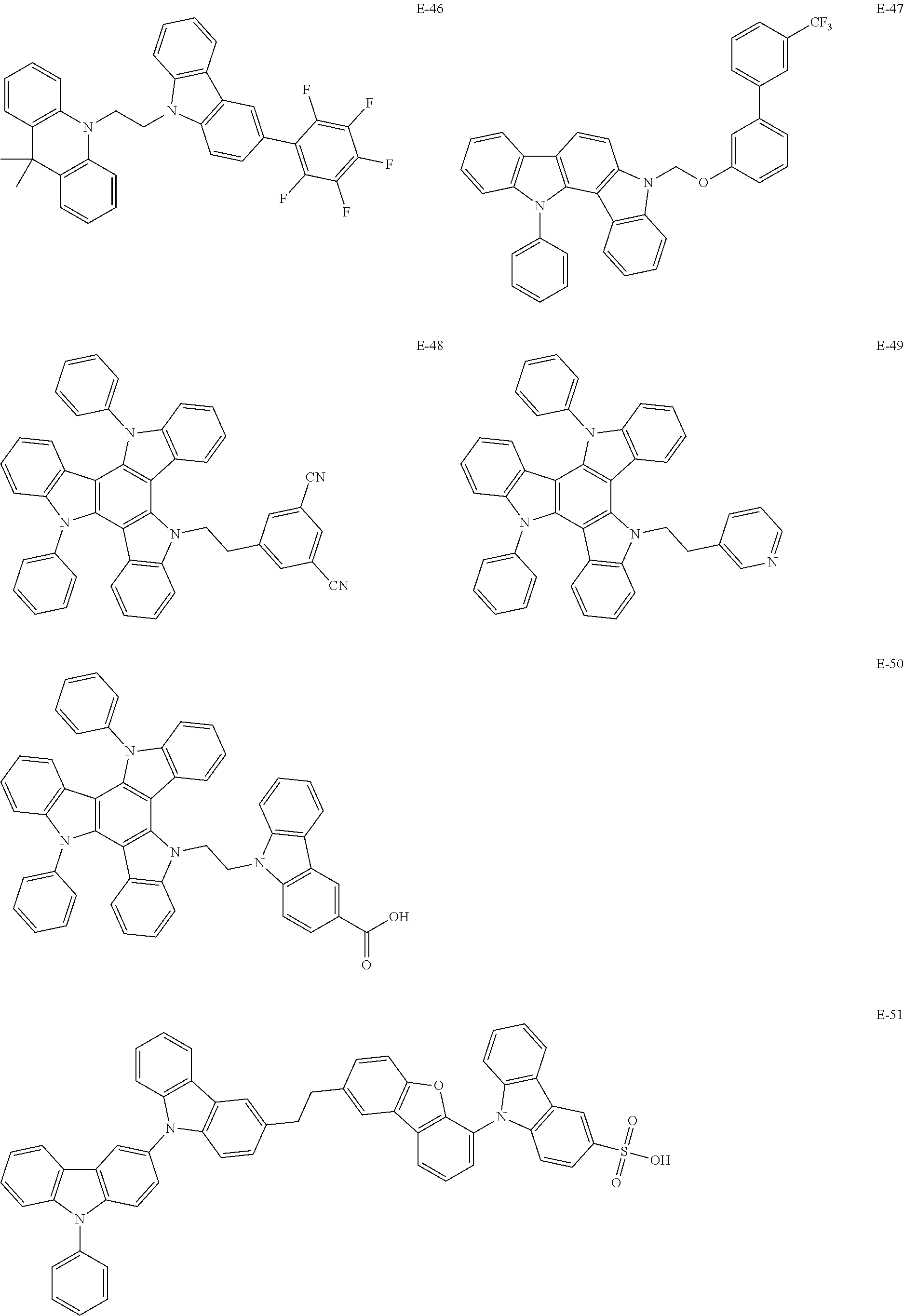

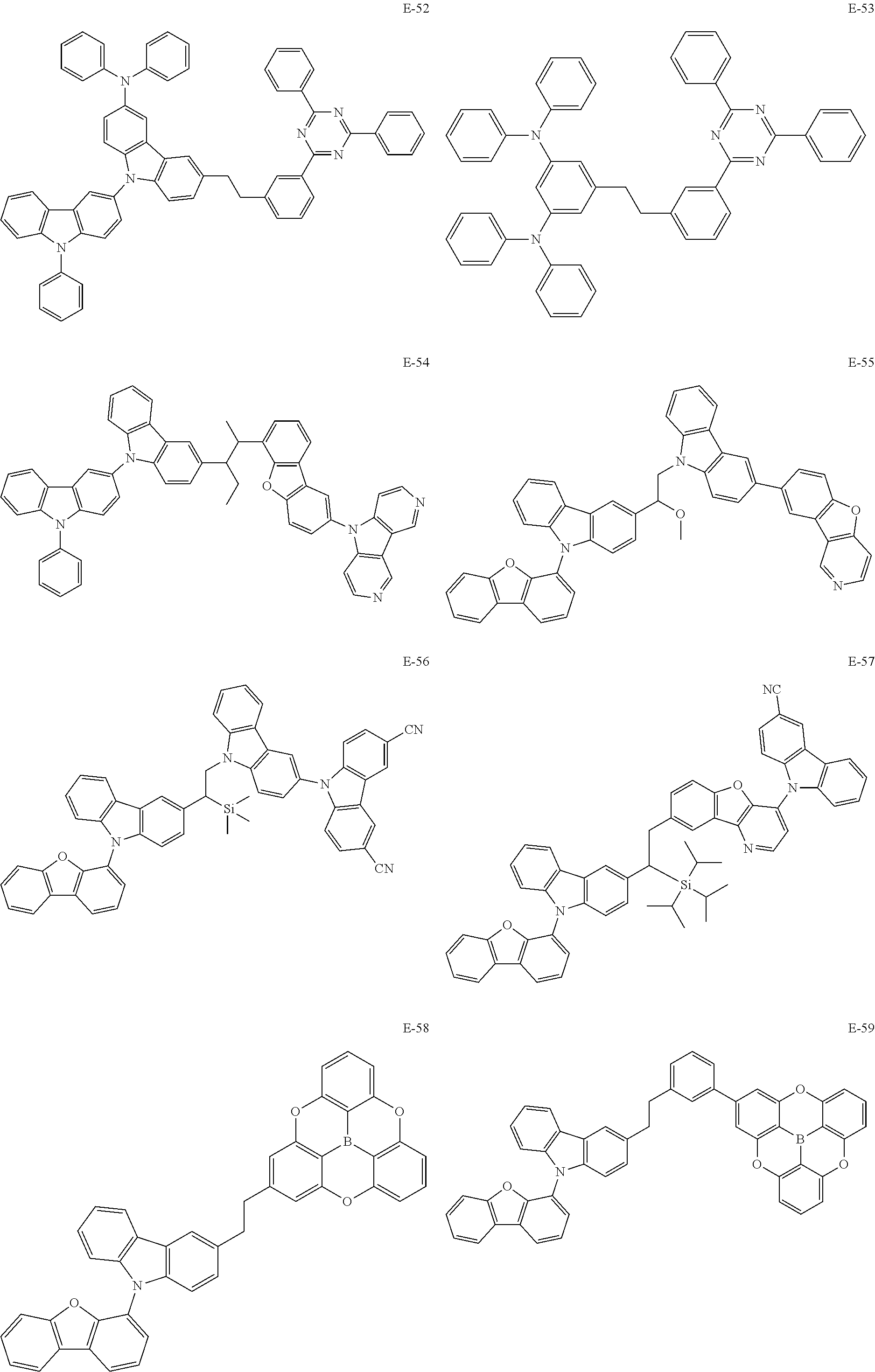

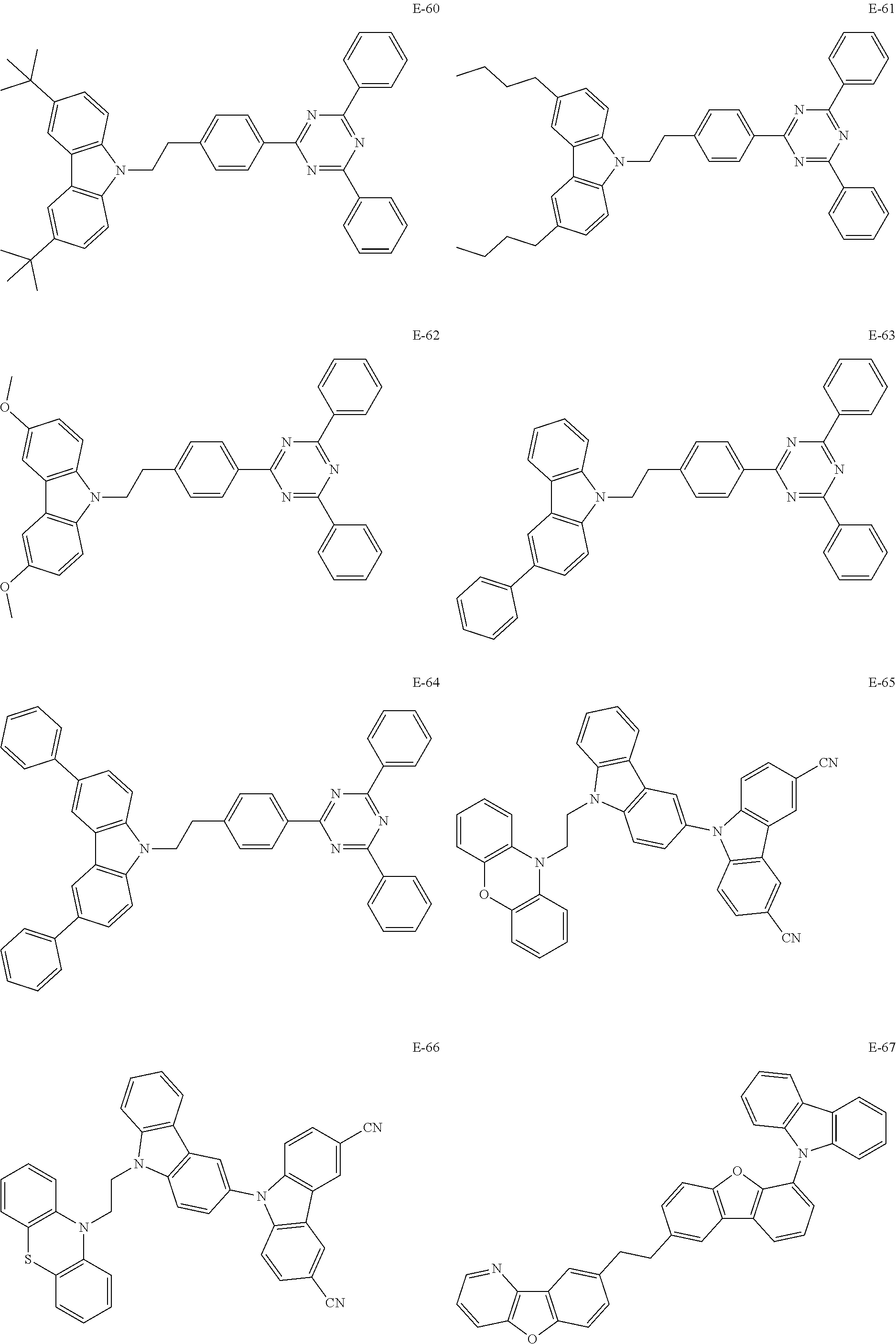

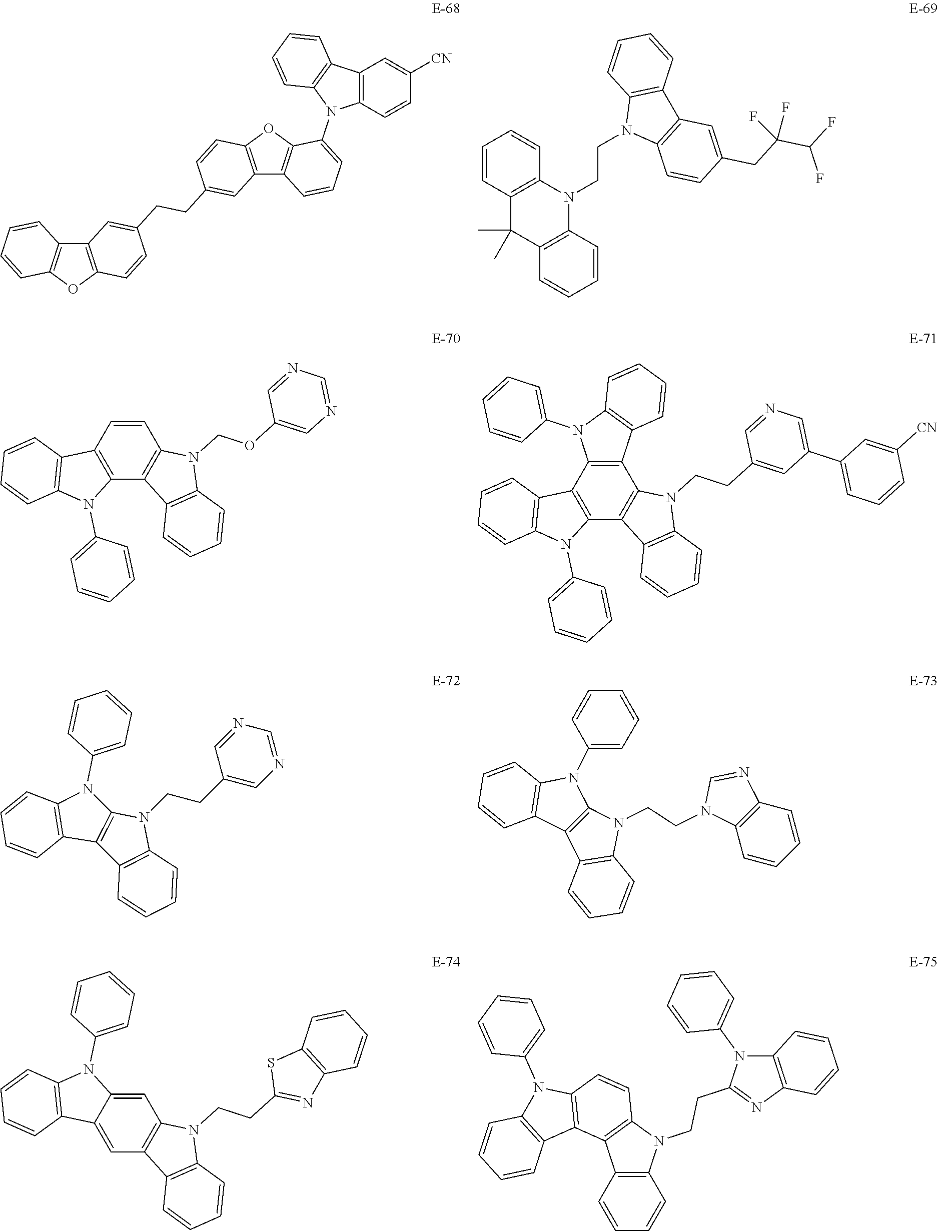

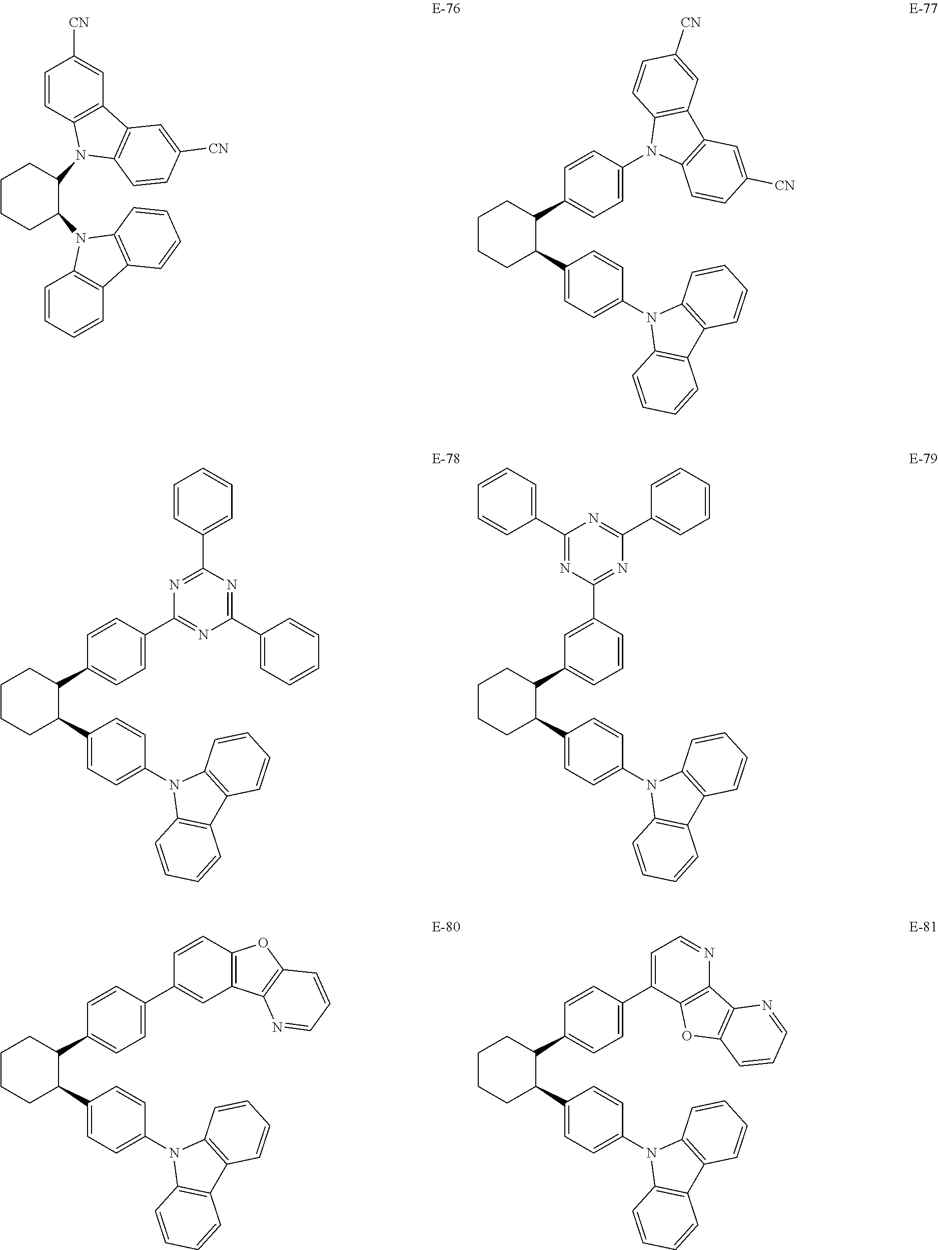

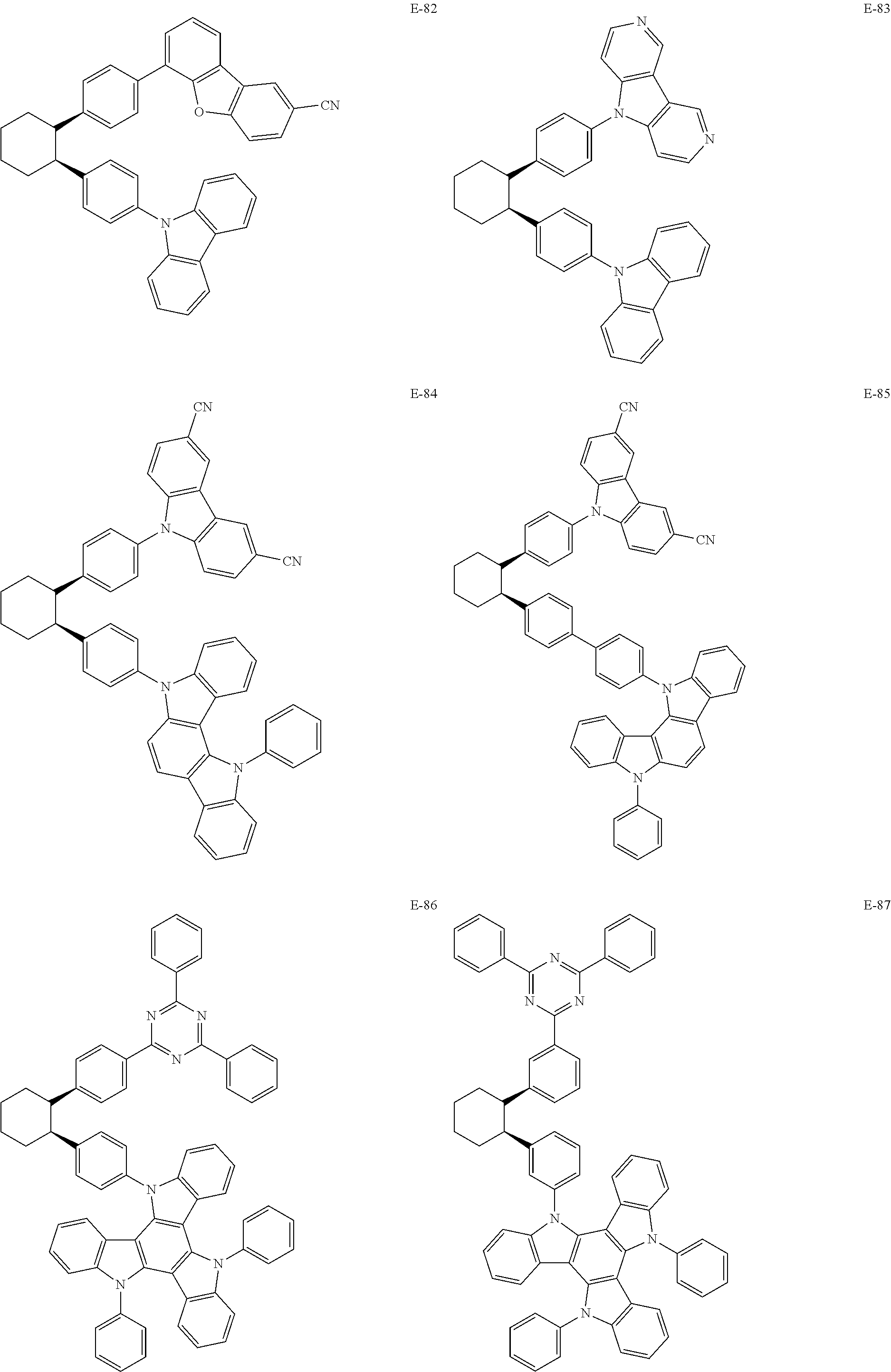

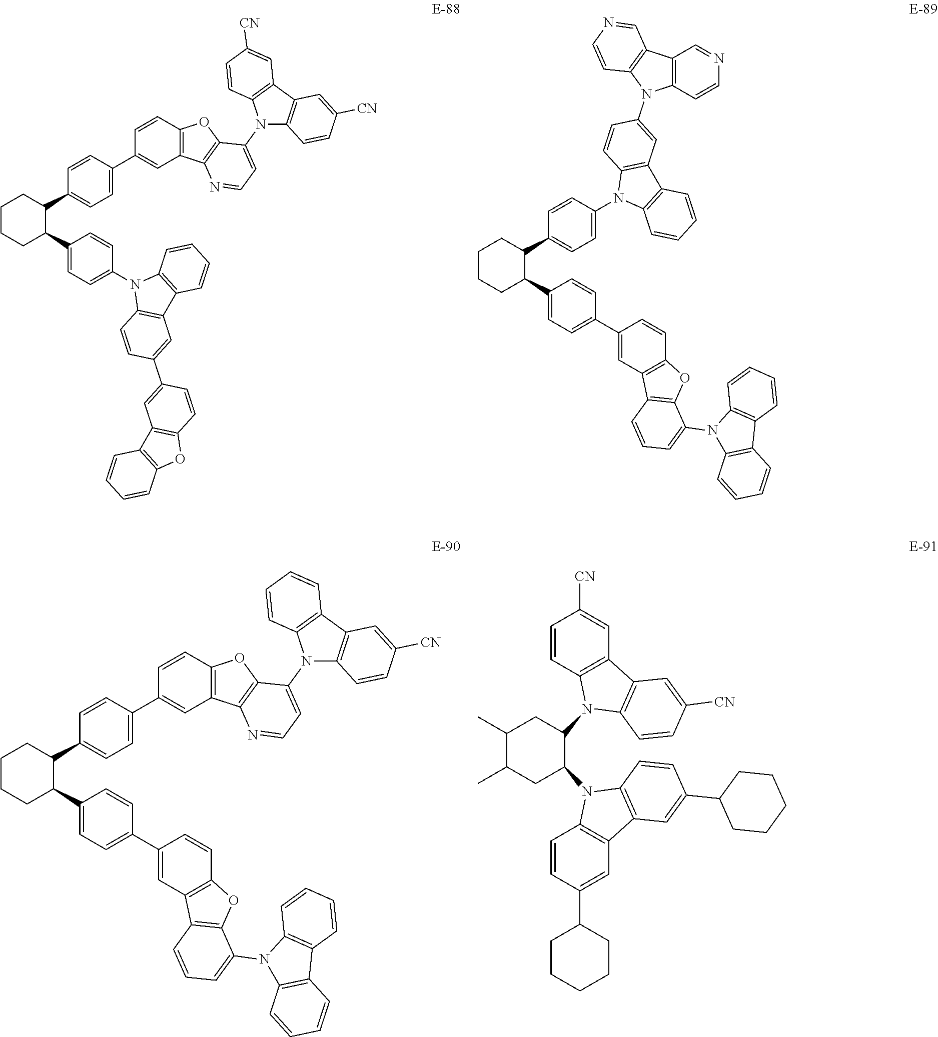

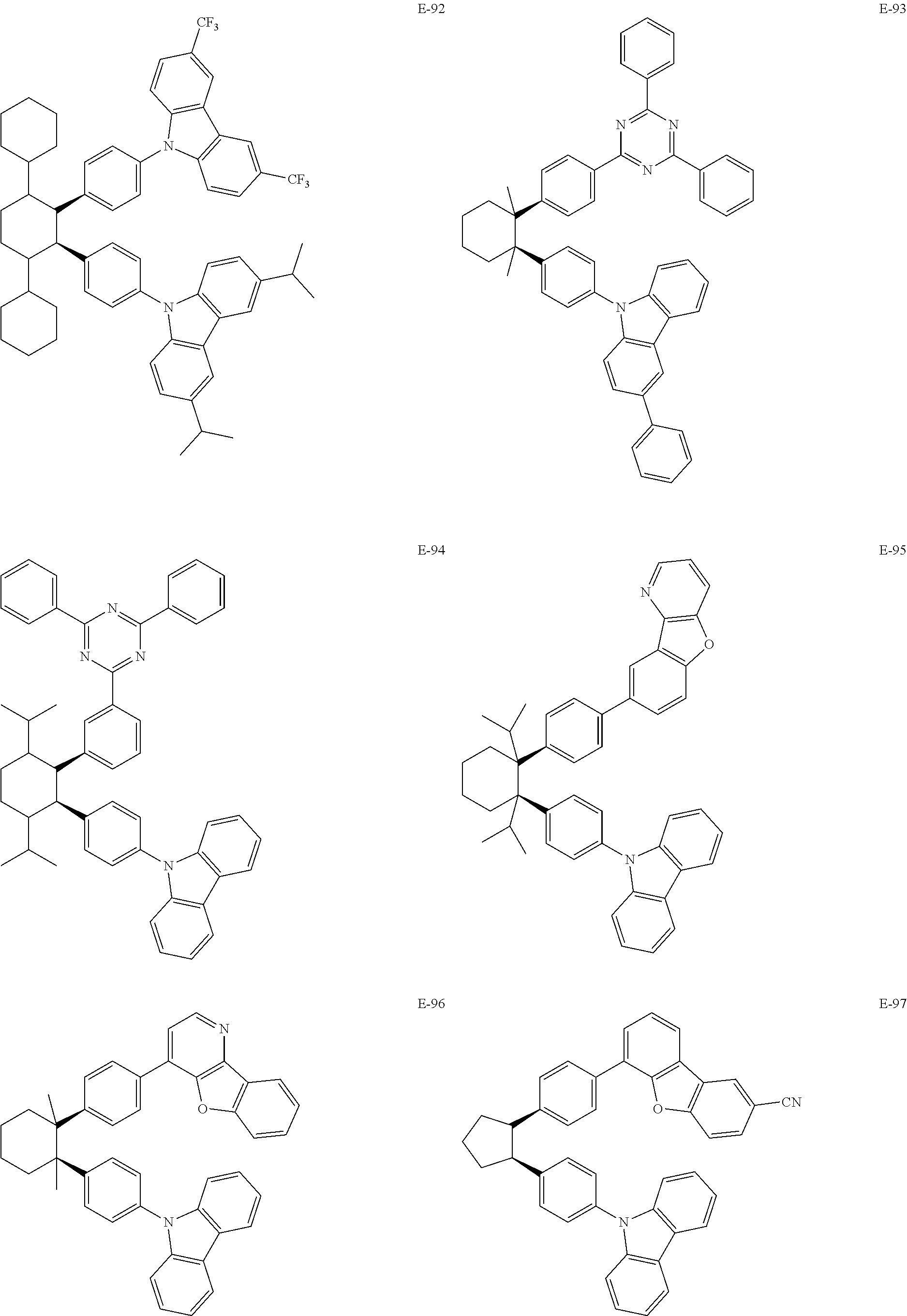

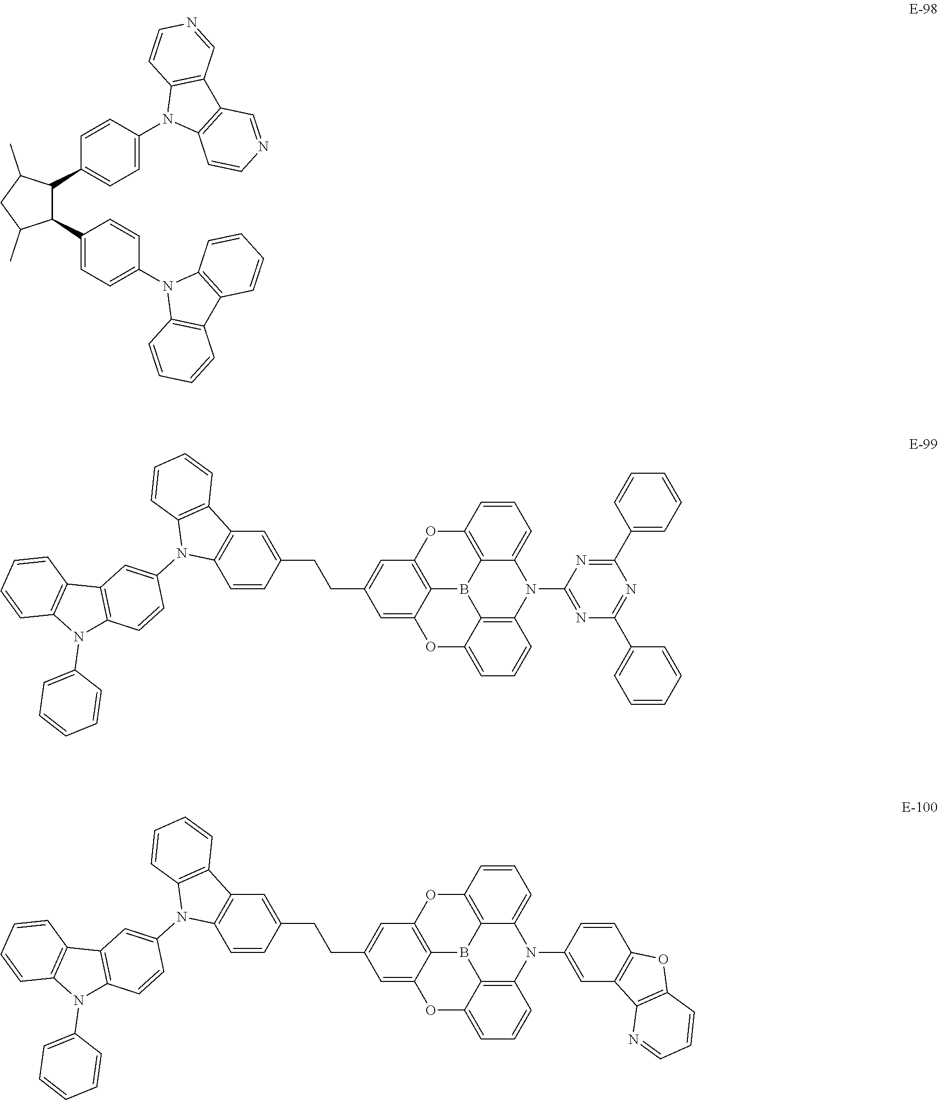

The molecular weight of the organic EL element material of the present invention is preferably in the range of 300 to 2000, and more preferably in the range of 400 to 900, from the viewpoint of enabling thin film formation.

By using the organic EL element material containing the compound having a structure represented by Formula (1) of the present invention, as described above, it is possible to form an exciton consisting of intramolecular or intermolecular exciplex while suppressing the increase in emission wavelength. Thus, an organic EL element containing an organic EL element material containing a compound having a structure represented by Formula (1) of the present invention exhibits a high luminous efficiency without greatly increasing the emission wavelength.

In Formula (1), a compound in which one is an electron donating group D represented by D and the other is an electron withdrawing group A represented by A forms an intramolecular or intermolecular exciplex. In such a compound that forms an intramolecular or intermolecular exciplex, an electron withdrawing group A interacts with an electron donating group D to form an exciton.

Whether a compound having a structure represented by Formula (1) of the present invention forms an intramolecular exciplex or does not form can be confirmed by the following method.
(1) The target compound is dissolved in 2-methyl-tetrahydrofuran to prepare a 1×10⁻⁵ M measuring solution. In the case of the compound represented by Formula (1) having both the electron withdrawing group A and the electron donating group D, the compound comprising the electron withdrawing group A, and the compound having the electron donating group D are dissolved in 2-methyl-tetrahydrofuran to prepare a 1×10⁻⁵ M comparative solution.
(2) The emission spectra of the measurement solution and the comparison solution are excited at their respective maximum absorption wavelengths and measured.
(3) The emission spectrum of the measurement solution and the emission spectrum of the comparison solution are superimposed.
(4) When the emission spectrum of the measurement solution and the emission spectrum of the comparison solution do not match, specifically, when the maximum emission wavelength of the emission spectrum of the measurement solution is shifted to the longer wavelength side than the maximum emission wavelength of the emission spectrum of the comparison solution, and the emission spectrum of the measurement solution is more than that of the comparison solution, and when the spectrum is also broad, it can be judged that the compound to be measured forms an intramolecular exciplex or an intramolecular excimer.

On the other hand, whether a compound having a structure represented by Formula (1) of the present invention forms an intermolecular exciplex or does not form can be confirmed by the following method.
(1) By using the above-described compound, a single layer film (hereinafter referred to as a single film) is produced by a deposition or coating process.
(2) The emission spectra of the measurement solution prepared by dissolving the above-mentioned target compound in 2-methyl-tetrahydrofuran and a single film are measured by exciting them at their respective maximum absorption wavelengths.
(3) The emission spectrum of the measurement solution and the emission spectrum of the single film are superimposed.
(4) When the emission spectrum of the measurement solution and the emission spectrum of the single film do not match, specifically, the maximum emission wavelength of the emission spectrum of the single film is shifted to the longer wavelength side than the emission wavelength of the emission spectrum of the measurement solution, and the emission spectrum of the single film is broader than the emission spectrum of the measurement solution, it can be judged that the compound to be measured forms an intermolecular exciplex.

Further, since these compounds have a bipolar property and they may be compatible with a variety of energy levels, they may be used as a light emitting compound and a host compound, and they may be suitably used as a hole transport compound or an electron transport compound. Namely, they may be used as a charge transport material. Consequently, the use of these compounds is not limited to a light emitting layer, they may be used in the hole injection layer, a hole transport layer, an electron blocking layer, a hole blocking layer, an electron transport layer, an electron injection layer, or an intermediate layer.

### [Synthesis of Compound Having a Structure Represented by Formula (1)]

The compound having a structure represented by Formula (1) may be synthesized, for example, by referring to the method described in WO 2014/022008 or the method described in the reference described in the same document.

An example of a synthesis example is indicated in the following.

### (Synthesis of Exemplified Compound E-1)

A synthetic flow of an exemplified compound E-1 having an ethylene linker (—CH₂—CH₂—) as XY is indicated below.

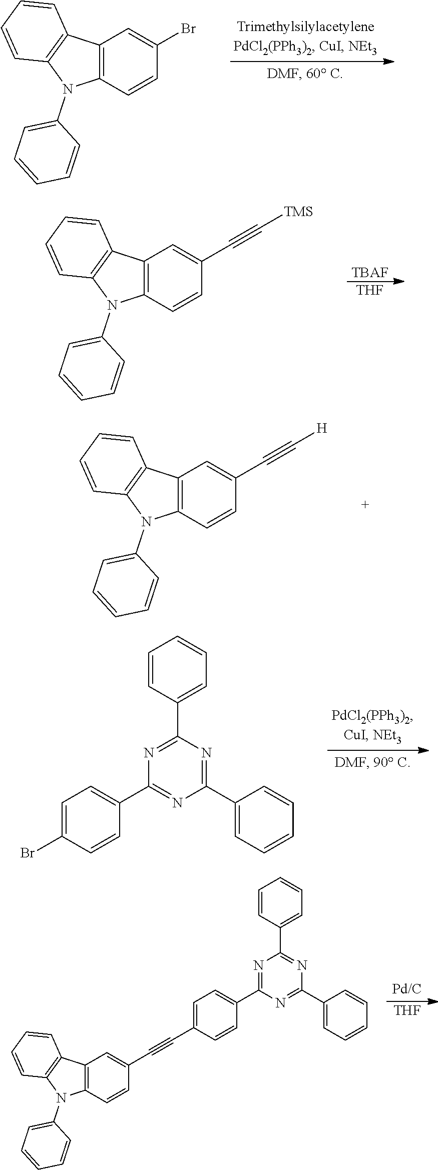

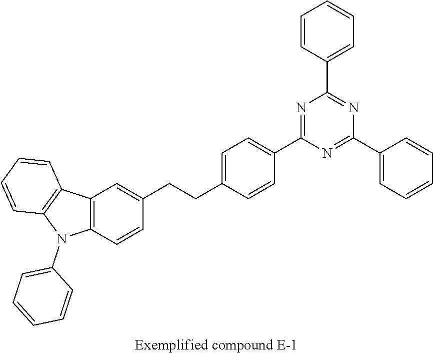 [SMILES: c1ccc(-c2nc(-c3ccccc3)nc(-c3ccc(CCc4ccc5c(c4)c4ccccc4n5-c4ccccc4)cc3)n2)cc1]

### (Synthesis of Exemplified Compound E-77)

A synthetic flow of an exemplified compound E-77 having a cyclohexyl linker as XY is indicated below.

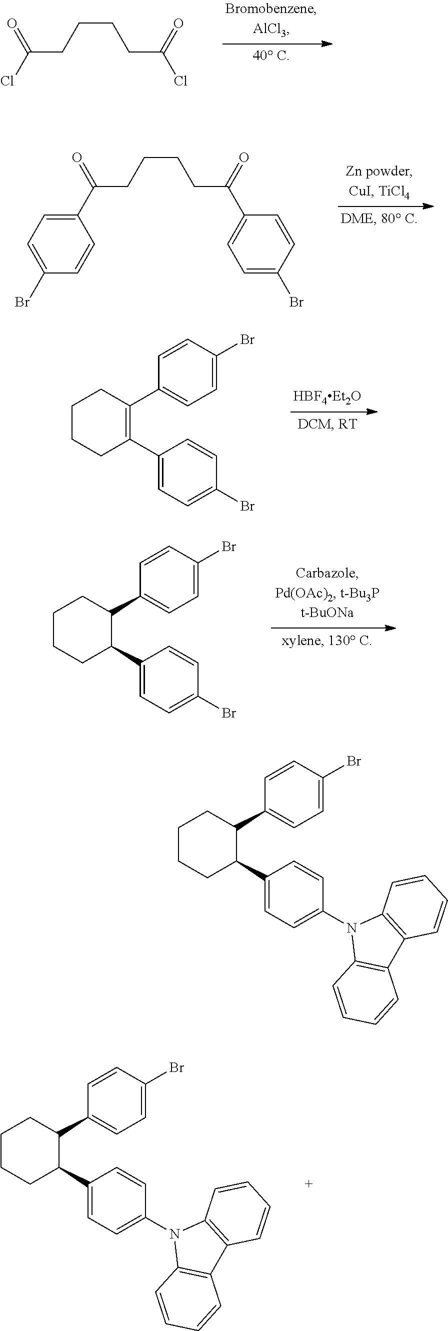

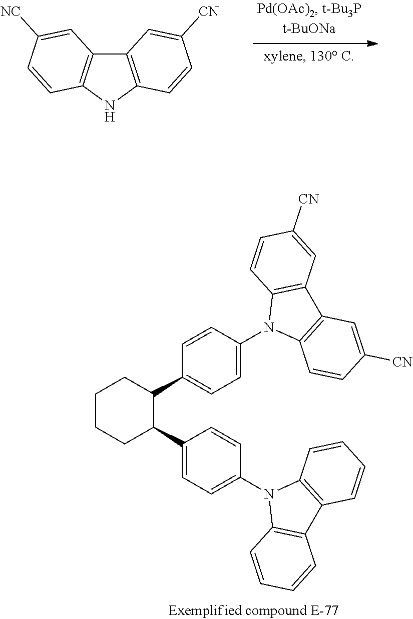

### <<Organic Electroluminescent Element>>

The compound having a structure represented by Formula (1) of the present invention forms an intramolecular or intermolecular exciplex with one type of molecule. This compound may be apply to an organic electroluminescent element (organic EL element). Specifically, the compound having a structure represented by Formula (1) according to the present invention may be used in applications such as a light emitting material, a host material, and an assist-dopant which constitute an organic EL element from the viewpoint of obtaining an organic EL element with high luminous efficiency.

### [Light Emitting Material]

### (Phosphorescence Emitting Compound)

As described above, although the phosphorescence emission is theoretically three times more advantageous than fluorescence emission as luminous efficiency, an energy deactivation (=phosphorescence emission) from the triplet excited state to the singlet ground state is a forbidden transition. In the same manner, the intersystem crossing from the singlet excited state to the triplet excited state is also a forbidden transition. Consequently, its rate constant is usually small. That is, since the transition takes place hardly, the lifetime of the exciton becomes long such as an order of millisecond or second. As a result, it is difficult to obtain a required light emission.

However, when a light emission occurs from a complex including a heavy atom of iridium or platinum, the rate constant of the above-described forbidden transition becomes larger by 3 orders due to the heavy metal effect of the center metal. It is possible to obtain a phosphorescence quantum efficiency of 100% when selection of the ligand is properly done.

However, in order to obtain an ideal emission, it is required to use a rare metal such as iridium or palladium, or a noble metal such as platinum. If a large amount of these metals are used, the reserves and the price of these metal will become problem.

### (Fluorescence Emitting Compound)

On the other hand, a common fluorescence emitting compound is not required to be a heavy metal complex as in the case of a phosphorescence emitting compound. It may be applied a so-called organic compound composed of a combination of elements such as carbon, oxygen, nitrogen and hydrogen. Further, a non-metallic element such as phosphor, sulfur, and silicon may be used. And a complex of typical element such as aluminum or zinc may be used. The variation of the materials is almost without limitation.

However, the conventional fluorescence emission material will use only 25% of the excitons to light emission as described above. Therefore, it cannot be expected high luminous efficiency as achieved in phosphorescence emission.

### <<Organic EL Element>>

The organic EL element of the present invention is configured to have at least a light emitting layer between an anode and a cathode. At least one layer of the light emitting layers contains a compound having a structure represented by Formula (1) which forms an intramolecular or intermolecular exciplex with one type of molecule.

Representative element configurations used for an organic EL element of the present invention are as follows, however, the present invention is not limited to these.
(1) Anode/light emitting layer/cathode
(2) Anode/light emitting layer/electron transport layer/cathode
(3) Anode/hole transport layer/light emitting layer/cathode
(4) Anode/hole transport layer/light emitting layer/electron transport layer/cathode
(5) Anode/hole transport layer/light emitting layer/electron transport layer/electron injection layer/cathode
(6) Anode/hole injection layer/hole transport layer/light emitting layer/electron transport layer/cathode
(7) Anode/hole injection layer/hole transport layer/(electron blocking layer)/light emitting layer/(hole blocking layer)/electron transport layer/electron injection layer/cathode

Among the above, the configuration shown in (7) is preferably used.

The light emitting layer constituting the organic EL element of the present invention is composed of one or a plurality of layers. When a plurality of layers are employed, a non-light emitting intermediate layer may be placed between the light emitting layers.

According to necessity, it may be provided with a hole blocking layer (it is also called as a hole barrier layer) or an electron injection layer (it is also called as a cathode buffer layer) between the light emitting layer and the cathode. Further, it may be provided with an electron blocking layer (it is also called as an electron barrier layer) or an hole injection layer (it is also called as an anode buffer layer) between the light emitting layer and the anode.

An electron transport layer used in the present invention is a layer having a function of transporting an electron. An electron transport layer includes an electron injection layer, and a hole blocking layer in a broad sense. Further, an electron transport layer unit may be composed of plural layers.

A hole transport layer used in the present invention is a layer having a function of transporting a hole. A hole transport layer includes a hole injection layer, and an electron blocking layer in a broad sense. Further, a hole transport layer unit may be composed of plural layers.

In the above-described representative configurations of the organic EL element, the layers eliminating an anode and a cathode are also called as “organic layers”, “organic functional layers” or “an organic functional layer group”.

### [Tandem Structure]

An organic EL element of the present invention may be so-called a tandem structure element in which plural light emitting units each containing at least one light emitting layer are laminated.

A representative example of an element configuration having a tandem structure is as follows.

Tandem Structure:

Anode/first light emitting unit/intermediate layer/second light emitting unit/intermediate layer/third light emitting unit/cathode.

Here, the above-described first light emitting unit, second light emitting unit, and third light emitting unit may be the same or different. It is possible that two light emitting units are the same and the remaining one light emitting unit is different.

The plural light emitting units each may be laminated directly or they may be laminated through an intermediate layer. Examples of an intermediate layer are: an intermediate electrode, an intermediate conductive layer, a charge generating layer, an electron extraction layer, a connecting layer, and an intermediate insulating layer. Known composing materials may be used as long as it can form a layer which has a function of supplying an electron to an adjacent layer to the anode, and a hole to an adjacent layer to the cathode.

Examples of a material used in an intermediate layer are: conductive inorganic compounds such as ITO (indium tin oxide), IZO (indium zinc oxide), ZnO₂, TiN, ZrN, HfN, TiO_{X}, VO_{X}, CuI, InN, GaN, CuAlO₂, CuGaO₂, SrCu₂O₂, LaB₆, RuO₂, and Al; a two-layer film such as Au/Bi₂O₃; a multi-layer film such as SnO₂/Ag/SnO₂, ZnO/Ag/ZnO, Bi₂O₃/Au/Bi₂O₃, TiO₂/TiN/TiO₂, and TiO₂/ZrN/TiO₂; fullerene such as C₆₀; and a conductive organic layer such as oligothiophene, metal phthalocyanine, metal-free phthalocyanine, metal porphyrin, and metal-free porphyrin. The present invention is not limited to them.

Examples of a preferable constitution in the light emitting unit are the configurations of the above-described (1) to (7) from which an anode and a cathode are removed. However, the present invention is not limited to them.

Examples of the configuration of the tandem type organic EL element are described in: U.S. Pat. Nos. 6,337,492, 7,420,203, 7,473,923, 6,872,472, 6,107,734, 6,337,492, WO 2005/009087, JP-A 2006-228712, JP-A 2006-24791, JP-A 2006-49393, JP-A 2006-49394, JP-A 2006-49396, JP-A 2011-96679, JP-A 2005-340187, JP Patent 4711424, JP Patent 3496681, JP Patent 3884564, JP Patent 4213169, JP-A 2010-192719, JP-A 2009-076929, JP-A 2008-078414, JP-A 2007-059848, JP-A 2003-272860, JP-A 2003-045676, and WO 2005/094130. The configurations of the element and the composing materials are described in these documents, however, the present invention is not limited to them.

### [Configuration of Organic EL Element]

Each layer that constitutes an organic EL element of the present invention will be described in the following.

### (Light Emitting Layer)

A light emitting layer constituting an organic EL element of the present invention is a layer which provide a place of emitting light via an exciton produce by recombination of electrons and holes injected from an electrode or an adjacent layer. The light emitting portion may be either within the light emitting layer or at an interface between the light emitting layer and an adjacent layer thereof. The configuration of the light emitting layer applied to the present invention is not particularly limited as long as the requirements specified in the present invention are satisfied.

The total thickness of the light emitting layer is not particularly limited, but from the viewpoint of achieving homogeneity of the film to be formed and preventing application of unnecessary high voltage at the time of light emission and improvement of stability of luminescent color with respect to driving current, it is preferable to adjust in the range of 2 nm to 5 μm, more preferably in the range of 2 nm to 500 nm, and further preferably in the range of 5 to 200 nm.

The thickness of each light emitting layer constituting an organic EL element of the present invention is preferably adjusted in the range of 2 nm to 1 μm, more preferably adjusted in the range of 2 to 200 nm, further preferably in the range of 3 to 150 nm.

The light emitting layer which forms the organic EL element of the present invention may be composed of one layer, and may be composed of a several layers. When the compound having a structure represented by Formula (1) of the present invention is applied to the light emitting layer, it may be used alone or mixed with a host material, a fluorescence emitting material, and a phosphorescence emitting material. It is preferable that at least one layer of the light emitting layers contains a light emitting dopant (a light emitting compound, a light emitting dopant, also simply referred to as a dopant), and further a host compound (a matrix material, a light emitting host compound, a host material, also simply referred to as a host). In the present invention, it is preferable that at least one layer of the light emitting layers contains a compound having a structure represented by Formula (1) of the present invention and a host compound, from the viewpoint of improving luminous efficiency. When at least one layer of the light emitting layers contains a compound having a structure represented by Formula (1) of the present invention, and at least one of a fluorescent compound and a phosphorescent compound, it is preferable in term of improving the luminous efficiency. Further, it is preferable that at least one layer of the light emitting layers contains a compound having a structure represented by Formula (1) of the present invention, at least one of a fluorescence emitting compound and a phosphorescent emitting compound, and a host compound from the viewpoint of improving the luminous efficiency.

### (1: Light Emitting Dopant)

As a light emitting dopant (hereafter, also referred to “a light emitting compound”), it is preferable to employ a fluorescence emitting dopant (also referred to as a fluorescence emitting compound and a fluorescent dopant) and a phosphorescence emitting dopant (also referred to as a phosphorescent emitting compound and a phosphorescent dopant). In the present invention, it is preferable that one of the light emitting layers contains a compound having a structure represented by Formula (1) of the present invention as an light emitting compound or an assist-dopant in an amount of 0.1 to 50 mass %, more preferably in an amount of 1 to 30 mass %

A concentration of a light emitting compound in a light emitting layer may be arbitrarily decided based on the specific compound employed and the required conditions of the device. A concentration of a light emitting compound may be uniform in a thickness direction of the light emitting layer, or it may have any concentration distribution.

The light emitting compound used in the present invention may be used in combination of two or more kinds A combination of fluorescence emitting compounds each having a different structure, a combination of phosphorescence emitting compounds each having a different structure, or a combination of a fluorescence emitting compound and a phosphorescence emitting compound may be used. Any required emission color will be obtained by this.

When the light emitting compound and the host compound are contained in the light emitting layer together with the compound having a structure represented by Formula (1) of the present invention, the compound having a structure represented by Formula (1) acts as an assist-dopant. On the other hand, when the light emitting layer contains the compound having a structure represented by Formula (1) of the present invention, and the light emitting compound, and does not contain the host compound, the compound having a structure represented by Formula (1) acts as a host compound. When the light emitting layer contains only the compound having a structure represented by Formula (1) of the present invention, the compound having a structure represented by Formula (1) acts as a host compound and a light emitting compound.

As a mechanism which expresses an effect, it is the same in either case. The absolute value (ΔE_{ST}) of the difference between the lowest excited singlet energy level and the lowest excited triplet energy level of the compound having a structure represented by Formula (1) of the present invention is at a minimum

By this, all energy of the excitons produced on the compound having a structure represented by Formula (1) of the present invention is theoretically transferred to the light emitting compound as fluorescence resonance energy transfer (FRET), as a result, it is possible to achieve high luminous efficiency.

Consequently, when the light emitting layer contains 3 components of a compound having a structure represented by Formula (1) of the present invention, a light emitting compound, and a host compound, it is preferable that the energy levels of S₁ and T₁ of the compound having a structure represented by Formula (1) are lower than the energy levels of S₁ and T₁ of the host compound, and higher than the energy levels of S₁ and T₁ of the light emitting compound.

In the same manner, when the light emitting layer contains 2 components of a compound having a structure represented by Formula (1) of the present invention and a light emitting compound, it is preferable that the energy levels of S₁ and T₁ of the compound having a structure represented by Formula (1) are higher than the energy levels of S₁ and T₁ of the light emitting compound.

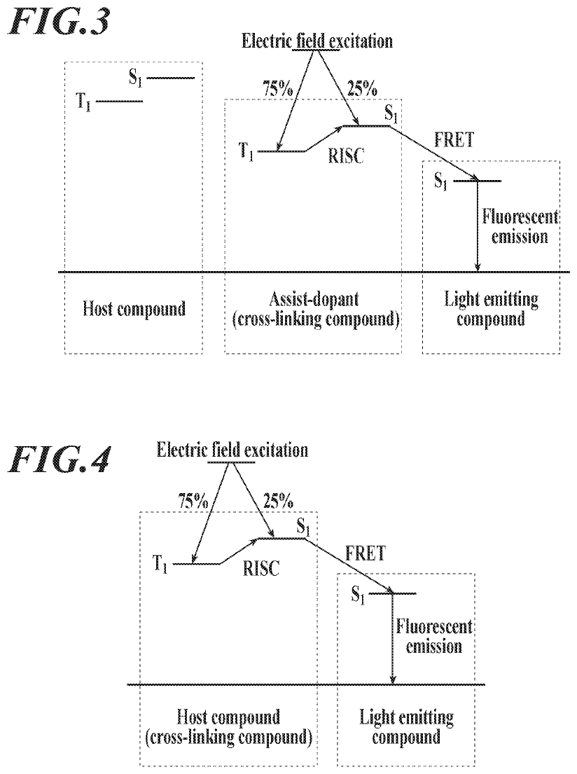

FIG. 3 and FIG. 4 illustrate a schematic diagram of the case in which the compound having a structure represented by Formula (1) of the present invention acts as an assist-dopant or a host compound.

FIG. 3 is a schematic view indicating an example of an energy diagram when the compound having a structure represented by Formula (1) functions as an assist-dopant. FIG. 4 is a schematic view indicating an example of an energy diagram when the compound having a structure represented by Formula (1) functions as a host compound.

FIG. 3 and FIG. 4 are only an example, the production process of the triplet exciton on the compound having a structure represented by Formula (1) of the present invention is not limited to the electric field excitation, the production process includes the cases of an energy transfer or an electron transfer in the light emitting layer or from the surrounding interface.

Further, FIG. 3 and FIG. 4 illustrate the diagram using a fluorescence emitting compound as a light emitting compound, however, the present invention is not limited to it, a phosphorescence emitting compound may be used, and both of a fluorescence emitting compound and a phosphorescence emitting compound may be used.

When the compound having a structure represented by Formula (1) of the present invention is used as an assist-dopant, it is preferable that the light emitting layer contains a host compound in an amount of 100 mass % or more with respect to 100 mass % of the compound having a structure represented by Formula (1), and that it contains a fluorescence emitting compound or a phosphorescence emitting compound in an amount of 0.1 to 50 mass % with respect to 100 mass % of the compound having a structure represented by Formula (1).

When the compound having a structure represented by Formula (1) of the present invention is used as a host compound, it is preferable that the light emitting layer contains a fluorescence emitting compound or a phosphorescence emitting compound in an amount of 0.1 to 50 mass % with respect to 100 mass % of the compound having a structure represented by Formula (1).

When the compound having a structure represented by Formula (1) of the present invention is used as an assist-dopant or a host compound, it is preferable that an emission spectrum of the compound having a structure represented by Formula (1) of the present invention and an absorption spectrum of the light emitting compound are overlapped.

Colors of light emitted by an organic EL element of the present invention or a compound used in the present invention are specified as follows. In FIG. 3.16 on page 108 of “Shinpen Shikisai Kagaku Handbook (New Edition Color Science Handbook)” (edited by The Color Science Association of Japan, Tokyo Daigaku Shuppan Kai, 1985), values determined via Spectroradiometer CS-1000 (produced by Konica Minolta, Inc.) are applied to the CIE chromaticity coordinate, whereby the color is specified.

In the present invention, it is also a preferable embodiment that one or more light emitting layers contain a plurality of light emitting dopants of different light emitting colors and exhibit white light emission.

The combination of luminescent dopants that produces white is not specifically limited. It may be cited, for example, combinations of: blue and orange; and blue, green and red.

It is preferable that “white” in the organic EL element of the present invention exhibits chromaticity in the CIE 1931 Color Specification System at 1,000 cd/m² in the region of x=0.39±0.09 and y=0.38±0.08, when measurement is done to 2-degree viewing angle front luminance via the aforesaid method.

### <1.1: Fluorescence Emitting Dopant>

As the fluorescence emitting dopant (hereinafter, also referred to as “fluorescent dopant”), a compound having a structure represented by Formula (1) of the present invention may be used. It may be appropriately selected and used from known fluorescent dopants and delayed fluorescent dopants used for forming the light emitting layer of the organic EL element.

Specific examples of known fluorescence emitting dopants applicable to the present invention are: an anthracene derivative, a pyrene derivative, a chrysene derivative, a fluoranthene derivative, a perylene derivative, a fluorene derivative, an arylacetylene derivative, a styrylarylene derivative, a styrylamine derivative, an arylamine derivative, a boron complex, a coumarin derivative, a pyran derivative, a cyanine derivative, a croconium derivative, a squarylium derivative, an oxobenzanthracene derivative, a fluorescein derivative, a rhodamine derivative, a pyrylium derivative, a perylene derivative, a polythiophene derivative, and a rare earth complex compound. In recent years, light emitting dopants utilizing delayed fluorescence were developed. These dopants may be used. Specific examples of a light emitting dopant utilizing delayed fluorescence are compounds described in: WO 2011/156793, JP-A 2011-213643, JP-A 2010-93181, Japanese Registered Patent 5,366,106. However, the present invention is not limited to them.

### <1.2: Phosphorescence Emitting Dopant>

A phosphorescence emitting applicable to the present invention will be described. Hereafter, it may be called as “a phosphorescent dopant”.

The phosphorescence emitting dopant is a compound which is observed emission from an excited triplet state thereof. Specifically, it is a compound which emits phosphorescence at a room temperature (25° C.) and exhibits a phosphorescence quantum yield of at least 0.01 at 25° C. The phosphorescence quantum yield is preferably at least 0.1.

The phosphorescence quantum yield will be determined via a method described in page 398 of Bunko II of Dai 4 Han Jikken Kagaku Koza 7 (Spectroscopy II of 4th Edition Lecture of Experimental Chemistry 7) (1992, published by Maruzen Co. Ltd.). The phosphorescence quantum yield in a solution will be determined using appropriate solvents. However, it is only necessary for the phosphorescent dopant of the present invention to exhibit the above phosphorescence quantum yield (0.01 or more) using any of the appropriate solvents.

A phosphorescent dopant may be suitably selected and employed from the known materials used for a light emitting layer for an organic EL element. Specific examples of a known phosphorescent dopant that may be used in the present invention are compounds described in the following publications.

Nature 395, 151 (1998), Appl. Phys. Lett. 78, 1622 (2001), Adv. Mater. 19, 739 (2007), Chem. Mater. 17, 3532 (2005), Adv. Mater. 17, 1059 (2005), WO 2009/100991, WO 2008/101842, WO 2003/040257, US 2006/835469, US 2006/0202194, US 2007/0087321, US 2005/0244673, Inorg. Chem. 40, 1704 (2001), Chem. Mater. 16, 2480 (2004), Adv. Mater. 16, 2003 (2004), Angew. Chem. Int. Ed. 2006, 45, 7800, Appl. Phys. Lett. 86, 153505 (2005), Chem. Lett. 34, 592 (2005), Chem. Commun. 2906 (2005), Inorg. Chem. 42, 1248 (2003), WO 2009/050290, WO 2002/015645, WO 2009/000673, US 2002/0034656, U.S. Pat. No. 7,332,232, US 2009/0108737, US 2009/0039776, U.S. Pat. Nos. 6,921,915, 6,687,266, US 2007/0190359, US 2006/0008670, US 2009/0165846, US 2008/0015355, U.S. Pat. Nos. 7,250,226, 7,396,598, US 2006/0263635, US 2003/0138657, US 2003/0152802, U.S. Pat. No. 7,090,928, Angew. Chem. Int. Ed. 47, 1 (2008), Chem. Mater. 18, 5119 (2006), Inorg. Chem. 46, 4308 (2007), Organometallics 23, 3745 (2004), Appl. Phys. Lett. 74, 1361 (1999), WO 2002/002714, WO 2006/009024, WO 2006/056418, WO 2005/019373, WO 2005/123873, WO 2005/123873, WO 2007/004380, WO 2006/082742, US 2006/0251923, US 2005/0260441, U.S. Pat. Nos. 7,393,599, 7,534,505, 7,445,855, US 2007/0190359, US 2008/0297033, U.S. Pat. No. 7,338,722, US 2002/0134984, and U.S. Pat. No. 7,279,704, US 2006/098120, US 2006/103874, WO 2005/076380, WO 2010/032663, WO 2008/140115, WO 2007/052431, WO 2011/134013, WO 2011/157339, WO 2010/086089, WO 2009/113646, WO 2012/020327, WO 2011/051404, WO 2011/004639, WO 2011/073149, JP-A 2012-069737, JP-A 2011-181303, JP-A 2009-114086, JP-A 2003-81988, JP-A 2002-302671 and JP-A 2002-363552.

Among them, preferable phosphorescent dopants are organic metal complexes containing Ir as a center metal. More preferable are complexes containing at least one coordination mode selected from a metal-carbon bond, a metal-nitrogen bond, a metal-oxygen bond and a metal-sulfur bond.

### (2: Host Compound)

A host compound used in the present invention is a compound which mainly plays a role of injecting or transporting a charge in a light emitting layer. In an organic EL element, an emission from the host compound itself is substantially not observed.

Among the compounds incorporated in the light emitting layer, a mass ratio of the host compound in the aforesaid layer is preferably 20 mass % or more.

A host compound may be used singly or may be used in combination of two or more compounds. By using plural host compounds, it is possible to adjust transfer of charge, thereby efficiency of an organic EL element may be improved.

In the following, preferable host compounds used in the present invention will be described.

A compound having a structure represented by Formula (1) of the present invention may be used as a host compound. However a known host compound may be used, and it is not specifically limited. From the viewpoint of a reverse energy transfer, it is preferable that the host compound has a larger excited energy level than an excited singlet energy level of the dopant compound. It is more preferable that the host compound has a larger excited triplet energy level than an excited triplet energy level of the dopant.

A host compound bears the function of transfer of the carrier and generation of an exciton in the light emitting layer. Therefore, it is preferable that the host compound will exist in all of the active species of a cation radical state, an anion radial state and an excited state, and that it will not make chemical reactions such as decomposition and addition. Further, it is preferable that the host molecule will not move in the layer with an Angstrom level when an electric current is applied.

In addition, in particular, when the light emitting dopant to be used in combination is a compound having a structure represented by Formula (1) of the present invention exhibiting exciplex emission, since the lifetime of the triplet excited state of the light emitting dopant is long, it is required an appropriate design of a molecular structure to prevent the host compound from having a lower T₁ level such as: the host compound has a high T₁ energy; the host compounds will not form a low T₁ state when aggregated each other; the light emitting dopant and the host compound will not form an exciplex; and the host compound will not form an electromer by applying an electric field.

In order to satisfy the above-described requirements, it is required that: the host compound itself has a high hopping mobility; the host compound has high hole hopping mobility; and the host compound has small structural change when it becomes a triplet excited state. As a representative host compound satisfying these requirements, preferable compounds are: a compound having a high T₁ energy such as a carbazole structure, an azacarbazole structure, a dibenzofuran structure, a dibenzothiophene structure and an azadibenzofuran structure.

A host compound is required to have a hole transporting ability or an electron transporting ability, and to prevent elongation of an emission wavelength. In addition, from the viewpoint of stably driving an organic EL element at high temperature, it is preferable that a host compound has a high glass transition temperature (Tg) of 90° C. or more, more preferably, has a Tg of 120° C. or more.

Here, a glass transition temperature (Tg) is a value obtained using DSC (Differential Scanning Colorimetry) based on the method in conformity to RS K 7121-2012.

Moreover, as a host compound used for the present invention, it is also suitable to use the compound having a structure represented Formula (1) of this invention. The compound having a structure represented Formula (1) according to the present invention has a high T₁ and it is suitably used for a light-emitting material having a short emission wavelength (that is, high energy levels of T₁ and S₁).

When a known host compound is used in the organic EL element of the present invention, specific examples thereof include compounds described in the following documents, but the present invention is not limited thereto.

Japanese patent application publication (JP-A) Nos. 2001-257076, 2002-308855, 2001-313179, 2002-319491, 2001-357977, 2002-334786, 2002-8860, 2002-334787, 2002-15871, 2002-334788, 2002-43056, 2002-334789, 2002-75645, 2002-338579, 2002-105445, 2002-343568, 2002-141173, 2002-352957, 2002-203683, 2002-363227, 2002-231453, 2003-3165, 2002-234888, 2003-27048, 2002-255934, 2002-260861, 2002-280183, 2002-299060, 2002-302516, 2002-305083, 2002-305084 and 2002-308837; US Patent Application Publication (US) Nos. 2003/0175553, 2006/0280965, 2005/0112407, 2009/0017330, 2009/0030202, 2005/0238919; WO 2001/039234, WO 2009/021126, WO 2008/056746, WO 2004/093 207, WO 2005/089025, WO 2007/063796, WO 2007/063754, WO 2004/107822, WO 2005/030900, WO 2006/114966, WO 2009/086028, WO 2009/003898, WO 2012/023947, JP-A 2008-074939, JP-A 2007-254297, and EP 2034538. Further, the compounds H-1 to H-230 described in paragraphs [0255] to [0293] of JP-A 2015-38941, and H-231 to H-234 described in the following may also be suitably used.

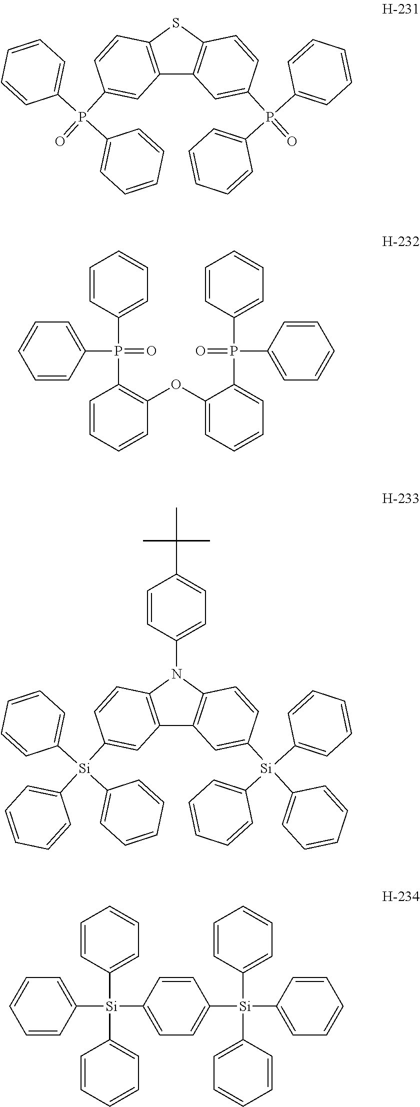 [SMILES: O=P(c1ccccc1)(c1ccccc1)c1ccc2sc3ccc(P(=O)(c4ccccc4)c4ccccc4)cc3c2c1 ; O=P(c1ccccc1)(c1ccccc1)c1ccccc1Oc1ccccc1P(=O)(c1ccccc1)c1ccccc1 ; CC(C)(C)c1ccc(-n2c3ccc([Si](c4ccccc4)(c4ccccc4)c4ccccc4)cc3c3cc([Si](c4ccccc4)(c4ccccc4)c4ccccc4)ccc32)cc1 ; c1ccc([Si](c2ccccc2)(c2ccccc2)c2ccc([Si](c3ccccc3)(c3ccccc3)c3ccccc3)cc2)cc1]

### (Electron Transport Layer)

An electron transport layer of the present invention is composed of a material having a function of transferring an electron. It is only required to have a function of transporting an injected electron from a cathode to a light emitting layer.

A total layer thickness of the electron transport layer according to the present invention is not specifically limited, however, it is generally in the range of 2 nm to 5 μm, and preferably, it is in the range of 2 to 500 nm, and more preferably, it is in the range of 5 to 200 nm.

In an organic EL element, it is known that there occurs interference between the light directly taken from the light emitting layer and the light reflected at the electrode located at the opposite side of the electrode from which the light is taken out at the moment of taking out the light which is produced in the light emitting layer. When the light is reflected at the cathode, it is possible to use effectively this interference effect by suitably adjusting the total thickness of the electron transport layer in the range of several nm to several μm.

On the other hand, the voltage will be increased when the layer thickness of the electron transport layer is made thick. Therefore, especially when the layer thickness is large, it is preferable that the electron mobility in the electron transport layer is 1×10⁻⁵ cm²/Vs or more.

As a material used for an electron transport layer (hereinafter, it is called as “an electron transport material”), it is only required to have either a property of ejection or transport of electrons, or a barrier to holes. Any of the conventionally known compounds may be selected and they may be employed.

Representative examples the electron transport material include: a nitrogen-containing aromatic heterocyclic derivative (a carbazole derivative, an azacarbazole derivative (a compound in which one or more carbon atoms constituting the carbazole ring are substitute with nitrogen atoms), a pyridine derivative, a pyrimidine derivative, a pyrazine derivative, a pyridazine derivative, a triazine derivative, a quinoline derivative, a quinoxaline derivative, a phenanthroline derivative, an azatriphenylene derivative, an oxazole derivative, a thiazole derivative, an oxadiazole derivative, a thiadiazole derivative, a triazole derivative, a benzimidazole derivative, a benzoxazole derivative, and a benzothiazole derivative); a dibenzofuran derivative, a dibenzothiophene derivative, a silole derivative; and an aromatic hydrocarbon ring derivative (a naphthalene derivative, an anthracene derivative and a triphenylene derivative).

Further, metal complexes having a ligand of a 8-quinolinol structure or dibnenzoquinolinol structure such as tris(8-quinolinol)aluminum (Alq₃), tris(5,7-dichloro-8-quinolinol)aluminum, tris(5,7-dibromo-8-quinolinol)aluminum, tris(2-methyl-8-quinolinol)aluminum, tris(5-methyl-8-quinolinol)aluminum and bis(8-quinolinol)zinc (Znq); and metal complexes in which a central metal of the aforesaid metal complexes is substituted by In, Mg, Cu, Ca, Sn, Ga or Pb, may be also utilized as an electron transport material.

Further, a metal-free or metal phthalocyanine, or a compound whose terminal is substituted by an alkyl group or a sulfonic acid group, may be preferably utilized as an electron transport material. A distyrylpyrazine derivative, which is exemplified as a material for a light emitting layer, may be used as an electron transport material. Further, in the same manner as used for a hole injection layer and a hole transport layer, an inorganic semiconductor such as an n-type Si and an n-type SiC may be also utilized as an electron transport material.

A polymer material which is introduced these compounds in the polymer side-chain or a polymer main chain may be used.

In an electron transport layer according to the present invention, it is possible to employ an electron transport layer of a higher n property (electron rich) which is doped with impurities as a guest material. As examples of a dope material, listed are those described in each of JP-A Nos. 4-297076, 10-270172, 2000-196140, 2001-102175, as well as in J. Appl. Phys., 95, 5773 (2004).

Although the present invention is not limited thereto, preferable examples of a known electron transport material used in an organic EL element of the present invention are compounds described in the following publications.

U.S. Pat. Nos. 6,528,187, 7,230,107, US 2005/0025993, US 2004/0036077, US 2009/0115316, US 2009/0101870, US 2009/0179554, WO 2003/060956, WO 2008/132085, Appl. Phys. Lett. 75, 4 (1999), Appl. Phys. Lett. 79, 449 (2001), Appl. Phys. Lett. 81, 162 (2002), Appl. Phys. Lett. 81, 162 (2002), Appl. Phys. Lett. 79, 156 (2001), U.S. Pat. No. 7,964,293, US 2009/030202, WO 2004/080975, WO 2004/063159, WO 2005/085387, WO 2006/067931, WO 2007/086552, WO 2008/114690, WO 2009/069442, WO 2009/066779, WO 2009/054253, WO 2011/086935, WO 2010/150593, WO 2010/047707, EP 2311826, JP-A 2010-251675, JP-A 2009-209133, JP-A 2009-124114, JP-A 2008-277810, JP-A 2006-156445, JP-A 2005-340122, JP-A 2003-45662, JP-A 2003-31367, JP-A 2003-282270, and WO 2012/115034.

In the present invention, more preferable known electron transport materials include aromatic heterocyclic compounds containing at least one nitrogen atom, and compounds containing a phosphorus atom. Examples thereof are: a pyridine derivative, a pyrimidine derivative, a pyrazine derivative, a triazine derivative, a dibenzofuran derivative, a dibenzothiophene derivative, an azadibenzofuran derivative, an azadibenzothiophene derivative, a carbazole derivative, an azacarbazole derivative, a benzimidazole derivative, and an aryl phosphine oxide derivative.

An electron transport material may be used singly, or may be used in combination of plural kinds of compounds.

### (Hole Blocking Layer)

A hole blocking layer is a layer provided with a function of an electron transport layer in a broad meaning. Preferably, it contains a material having a function of transporting an electron, and having very small ability of transporting a hole. It will improve the recombination probability of an electron and a hole by blocking a hole while transporting an electron.

Further, a composition of an electron transport layer described above may be appropriately utilized as a hole blocking layer of the present invention when needed.

A hole blocking layer placed in an organic EL element according to the present invention is preferably arranged at a location in the light emitting layer adjacent to the cathode side.

A thickness of a hole blocking layer according to the present invention is preferably in the range of 3 to 100 nm, and more preferably, in the range of 5 to 30 nm.

With respect to a material used for a hole blocking layer, the material used in the aforesaid electron transport layer is suitably used, and further, the material used as the aforesaid host compound is also suitably used for a hole blocking layer.

### (Electron Injection Layer)

An electron injection layer (it is also called as “a cathode buffer layer”) according to the present invention is a layer which is arranged between a cathode and a light emitting layer to decrease an operating voltage and to improve a light emission luminance An example of an electron injection layer is detailed in volume 2, chapter 2 “Electrode materials” (pp. 123-166) of “Organic EL Elements and Industrialization Front thereof (Nov. 30, 1998, published by N.T.S. Co. Ltd.)”.

In an organic EL element of the present invention, an electron injection layer is provided according to necessity, and as described above, it is placed between a cathode and a light emitting layer, or between a cathode and an electron transport layer.

An electron injection layer is preferably a very thin layer. The layer thickness is preferably in the range of 0.1 to 5 nm, depending on the material used for the formation. In addition, it may be a non-uniform island layer (film) in which constituent materials are discontinuously present.

An election injection layer is detailed in JP-A Nos. 6-325871, 9-17574, and 10-74586. Examples of a material preferably used in an election injection layer include: a metal such as strontium and aluminum; an alkaline metal compound such as lithium fluoride, sodium fluoride, or potassium fluoride; an alkaline earth metal compound such as magnesium fluoride; a metal oxide such as aluminum oxide; and a metal complex such as lithium 8-hydroxyquinolate (abbreviation: Liq). It is possible to use the aforesaid electron transport materials.

The above-described materials may be used singly or plural kinds may be used together in an election injection layer.

### (Hole Transport Layer)

A hole transport layer in an organic EL element of the present invention contains a material having a function of transporting a hole. A hole transport layer is only required to have a function of transporting a hole injected from an anode to a light emitting layer.

In the present invention, the total layer thickness of a hole transport layer is not specifically limited, however, it is generally in the range of 5 nm to 5 μm, preferably in the range of 2 to 500 nm, and more preferably in the range of 5 nm to 200 nm.

A material used in a hole transport layer (hereinafter, it is called as “a hole transport material”) is only required to have any one of properties of injecting and transporting a hole, and a barrier property to an electron. A hole transport material may be suitably selected from the conventionally known compounds.

Examples of the hole transport material include: a porphyrin derivative, a phthalocyanine derivative, an oxazole derivative, an oxadiazole derivative, a triazole derivative, an imidazole derivative, a pyrazoline derivative, a pyrazolone derivative, a phenylenediamine derivative, a hydrazone derivative, a stilbene derivative, a polyarylalkane derivative, a triarylamine derivative, a carbazole derivative, an indolocarbazole derivative, an isoindole derivative, an acene derivative of anthracene or naphthalene, a fluorene derivative, a fluorenone derivative, polyvinyl carbazole, a polymer or an oligomer containing an aromatic amine in a side chain or a main chain, polysilane, and a conductive polymer or an oligomer (e.g., PEDOT/PSS (poly 3,4-ethylene dioxythiophene/polystyrene sulfonic acid), an aniline type copolymer, polyaniline and polythiophene).

Examples of a triarylamine derivative include: a benzidine type represented by (4,4′-Bis[N-(1-naphthyl)-N-phenylamino]biphenyl) (abbreviation: α-NPD), a star burst type represented (4,4′,4″-Tris[N-(3-methylphenyl)-N-phenylamino]triphenylamine) (abbreviation: MTDATA,), and a compound having fluorenone or anthracene in a triarylamine bonding core.

A hexaazatriphenylene derivative described in JP-A Nos. 2003-519432 and 2006-135145 may be also used as a hole transport material.

In addition, it is possible to employ an electron transport layer of a higher p property which is doped with impurities. As its example, listed are those described in each of JP-A Nos. 4-297076, 2000-196140, and 2001-102175, as well as in J. Appl. Phys., 95, 5773 (2004).

Further, it is possible to employ so-called p-type hole transport materials, and inorganic compounds such as p-type Si and p-type SiC, as described in JP-A No. 11-251067, and J. Huang et al. reference (Applied Physics Letters 80 (2002), p. 139). Moreover, an orthometal compounds having Ir or Pt as a center metal represented by Ir(ppy)₃ are also preferably used.

Although the above-described compounds may be used as a hole transport material, preferably used are: a triarylamine derivative, a carbazole derivative, an indolocarbazole derivative, an azatriphenylene derivative, an organic metal complex, a polymer or an oligomer incorporated an aromatic amine in a main chain or in a side chain.

Specific examples of a known hole transport material used in an organic EL element of the present invention are compounds in the aforesaid publications and in the following publications. However, the present invention is not limited to them.

Examples of the publication are: Appl. Phys. Lett. 69, 2160(1996), J. Lumin. 72-74, 985(1997), Appl. Phys. Lett. 78, 673(2001), Appl. Phys. Lett. 90, 183503(2007), Appl. Phys. Lett. 51, 913(1987), Synth. Met. 87, 171(1997), Synth. Met. 91, 209(1997), Synth. Met. 111, 421(2000), SID Symposium Digest, 37, 923(2006), J. Mater. Chem. 3, 319(1993), Adv. Mater. 6, 677(1994), Chem. Mater. 15, 3148(2003), US 2003/0162053, US 2002/0158242, US 2006/0240279, US 2008/0220265, U.S. Pat. No. 5,061,569, WO 2007/002683, WO 2009/018009, EP 650955, US 2008/0124572, US 2007/0278938, US 2008/0106190, US 2008/0018221, WO 2012/115034, JP-A 2003-519432, JP-A 2006-135145, and U.S. patent application Ser. No. 13/585,981.

A hole transport material may be used singly or may be used in combination of plural kinds of compounds.

### (Electron Blocking Layer)

An electron blocking layer is a layer provided with a function of a hole transport layer in a broad meaning Preferably, it contains a material having a function of transporting a hole, and having very small ability of transporting an electron. It will improve the recombination probability of an electron and a hole by blocking an electron while transporting a hole.

Further, a composition of a hole transport layer described above may be appropriately utilized as an electron blocking layer of an organic EL element when needed.

In the organic EL element of the present invention, the electron blocking layer is preferably provided adjacent to the anode surface side of the light emitting layer.

In the present invention, a thickness of an electron blocking layer is preferably in the range of 3 to 100 nm, and more preferably, it is in the range of 5 to 30 nm.

With respect to a material used for an electron blocking layer, the material used in the aforesaid hole transport layer is suitably used, and further, the material used as the aforesaid host compound is also suitably used for an electron blocking layer.

### (Hole Injection Layer)

A hole injection layer (it is also called as “an anode buffer layer”) according to the present invention is a layer which is arranged between an anode and a light emitting layer to decrease an operating voltage and to improve a light emission luminance An example of a hole injection layer is detailed in volume 2, chapter 2 “Electrode materials” (pp. 123-166) of “Organic EL Elements and Industrialization Front thereof (Nov. 30, 1998, published by N.T.S. Co. Ltd.)”.

In an organic EL element of the present invention, a hole injection layer is provided according to necessity, and as described above, it is placed between an anode and a light emitting layer, or between an anode and a hole transport layer.

A hole injection layer is also detailed in JP-A Nos. 9-45479, 9-260062 and 8-288069. As materials used in the hole injection layer, it is cited the same materials used in the aforesaid hole transport layer.

Among them, preferable materials are: a phthalocyanine derivative represented by copper phthalocyanine; a hexaazatriphenylene derivative described in JP-A Nos. 2003-519432 and 2006-135145; a metal oxide represented by vanadium oxide; a conductive polymer such as amorphous carbon, polyaniline (or called as emeraldine) and polythiophene; an orthometalated complex represented by tris(2-phenylpyridine) iridium complex; and a triarylamine derivative.

The above-described materials used in a hole injection layer may be used singly or plural kinds may be co-used.

### (Additives in Organic EL Element)

Various additives may be contained in each organic functional layer group constituting the organic EL element of the present invention as required.

Examples of an additive are: halogen elements such as bromine, iodine and chlorine, and a halide compound; and a compound, a complex and a salt of an alkali metal, an alkaline earth metal and a transition metal such as Pd, Ca and Na.

Although a content of the additive may be arbitrarily decided, preferably, it is 1,000 ppm or less based on the total mass of the layer containing the ingredient, more preferably, it is 500 ppm or less, and still more preferably, it is 50 ppm or less.

In order to improve a transporting property of an electron or a hole, or to facilitate energy transport of an exciton, the content of the additive is not necessarily within these range, and other range of content may be used.

### (Method of Forming Each Organic Functional Layer)

Each organic functional layer (for example, a hole injection layer, a hole transport layer, a light emitting layer, a hole blocking layer, an electron transport layer, and an electron injection layer, and an intermediate layer) constituting the organic EL element of the present invention will be described.

The forming method of each organic functional layer constituting the organic EL element used in the present invention is not particularly limited. They may be formed by using a known method such as a vacuum vapor deposition method and a wet method (it may be called as a wet process).

Examples of a wet process include: a spin coating method, a cast method, an inkjet method, a printing method, a die coating method, a blade coating method, a roll coating method, a spray coating method, a curtain coating method, and a LB method (Langmuir Blodgett method). From the viewpoint of getting a uniform thin layer with high productivity, preferable are method highly appropriate to a roll-to-roll method such as a die coating method, a roll coating method, an inkjet method, and a spray coating method.

In the wet method, when preparing a coating solution for forming an organic functional layer, examples of a medium for dissolving or dispersing the organic EL material are: ketones such as methyl ethyl ketone and cyclohexanone; aliphatic esters such as ethyl acetate; halogenated hydrocarbons such as dichlorobenzene; aromatic hydrocarbons such as toluene, xylene, mesitylene, and cyclohexylbenzene; aliphatic hydrocarbons such as cyclohexane, decalin, and dodecane; organic solvents such as DMF (N,N-dimethylformamide) and DMSO (dimethyl sulfoxide).

These will be dispersed with a dispersion method such as an ultrasonic dispersion method, a high shearing dispersion method and a media dispersion method.

On the other hand, when a vapor deposition method is adopted for forming each layer, the vapor deposition conditions may be changed depending on the compounds used. Generally, the following ranges are suitably selected for the conditions, heating temperature of boat: 50 to 450° C., level of vacuum: 1×10⁻⁶ to 1×10⁻² Pa, vapor deposition rate: 0.01 to 50 nm/sec, temperature of substrate: −50 to 300° C., and layer thickness: 0.1 nm to 5 μm, preferably 5 to 200 nm.

Formation of organic functional layers according to the present invention is preferably continuously carried out from a hole injection layer to a cathode with one time vacuuming. It may be taken out on the way, and a different layer forming method may be employed. In that case, the operation is preferably done under a dry inert gas atmosphere.

### (Anode)

As an anode of an organic EL element, a metal having a large work function (4 eV or more, preferably, 4.5 eV or more), an alloy, and a conductive compound and a mixture thereof are utilized as an electrode substance. Specific examples of an electrode substance are: metals such as Au, and an alloy thereof; transparent conductive materials such as CuI, indium tin oxide (ITO), SnO₂, and ZnO. Further, a material such as IDIXO (In₂O₃—ZnO), which may form an amorphous and transparent electrode, may also be used.

As for an anode, these electrode substances may be made into a thin layer by a method such as a vapor deposition method or a sputtering method; followed by making a pattern of a desired form by a photolithography method. Otherwise, when the requirement of pattern precision is not so severe (about 100 μm or more), a pattern may be formed through a mask of a desired form at the time of layer formation with a vapor deposition method or a sputtering method using the above-described material.

Alternatively, when a coatable substance such as an organic conductive compound is employed, it is possible to employ a wet film forming method such as a printing method or a coating method. When emitted light is taken out from the anode, the transmittance is preferably set to be 10% or more. A sheet resistance of the anode is preferably a few hundred Ω/sq or less.

Although a layer thickness of the anode depends on a material, it is generally selected in the range of 10 nm to 1 μm, and preferably in the range of 10 to 200 nm.

### (Cathode)

As a cathode, a metal having a small work function (4 eV or less) (it is called as an electron injective metal), an alloy, a conductive compound and a mixture thereof are utilized as an electrode substance. Specific examples of the aforesaid electrode substance includes: sodium, sodium-potassium alloy, magnesium, lithium, a magnesium/copper mixture, a magnesium/silver mixture, a magnesium/aluminum mixture, a magnesium/indium mixture, an aluminum/aluminum oxide (Al₂O₃) mixture, indium, a lithium/aluminum mixture, aluminum, and a rare earth metal. Among them, with respect to an electron injection property and durability against oxidation, preferable are: a mixture of election injecting metal with a second metal which is stable metal having a work function larger than the electron injecting metal. Examples thereof are: a magnesium/silver mixture, a magnesium/aluminum mixture, a magnesium/indium mixture, an aluminum/aluminum oxide (Al₂O₃) mixture, a lithium/aluminum mixture and aluminum.

A cathode may be made by using these electrode substances with a method such as a vapor deposition method or a sputtering method to form a thin film. A sheet resistance of the cathode is preferably a few hundred Ω/sq or less. A layer thickness of the cathode is generally selected in the range of 10 nm to 5 μm, and preferably in the range of 50 to 200 nm.

In order to transmit emitted light, it is preferable that one of an anode and a cathode of an organic EL element is transparent or translucent for achieving an improved luminescence.

Further, after forming a layer of the aforesaid metal having a thickness of 1 to 20 nm on the cathode, it is possible to prepare a transparent or translucent cathode by providing with a conductive transparent material described in the description for the anode thereon. By applying this process, it is possible to produce an element in which both an anode and a cathode are transparent.

### [Support Substrate]

A support substrate which may be used for an organic EL element of the present invention is not specifically limited with respect to types such as glass and plastics. Hereinafter, the support substrate may be also called as substrate body, substrate, substrate substance, or support. They may be transparent or opaque. However, a transparent support substrate is preferable when the emitting light is taken from the side of the support substrate. Support substrates preferably utilized includes such as glass, quartz and transparent resin film. A specifically preferable support substrate is a resin film capable of providing an organic EL element with a flexible property.

Examples of a resin film include: polyesters such as polyethylene terephthalate (PET) and polyethylene naphthalate (PEN), polyethylene, polypropylene, cellophane, cellulose esters and their derivatives such as cellulose diacetate, cellulose triacetate (TAC), cellulose acetate butyrate, cellulose acetate propionate (CAP), cellulose acetate phthalate, and cellulose nitrate, polyvinylidene chloride, polyvinyl alcohol, polyethylene vinyl alcohol, syndiotactic polystyrene, polycarbonate, norbornene resin, polymethyl pentene, polyether ketone, polyimide, polyether sulfone (PES), polyphenylene sulfide, polysulfones, polyether imide, polyether ketone imide, polyamide, fluororesin, Nylon, polymethyl methacrylate, acrylic resin, polyallylates and cycloolefin resins such as ARTON (trade name, made by JSR Co. Ltd.) and APEL (trade name, made by Mitsui Chemicals, Inc.).

On the surface of a resin film, it may be formed a barrier film incorporating an inorganic or an organic compound or a hybrid film incorporating both compounds. Barrier films are preferred with a water vapor permeability of 0.01 g/(m²·24 h) or less (at 25±0.5° C., and 90±2% RH) determined based on JIS K 7129-1992. Further, high barrier films are preferred to have an oxygen permeability of 1×10⁻³ mL/(m²·24 h atm) or less determined based on JIS K 7126-1987, and a water vapor permeability 1 of 1×10⁻⁵ g/(m²·24 h) or less.

As materials that form a barrier film, employed may be those which retard penetration of moisture and oxygen, which deteriorate the element. For example, it is possible to employ silicon oxide, silicon dioxide, and silicon nitride. Further, in order to improve the brittleness of the aforesaid film, it is more preferable to achieve a laminated layer structure of inorganic layers and organic layers. The laminating order of the inorganic layer and the organic layer is not particularly limited, but it is preferable that both are alternatively laminated a plurality of times.

Barrier film forming methods are not particularly limited. Examples of employable methods include a vacuum deposition method, a sputtering method, a reactive sputtering method, a molecular beam epitaxy method, a cluster ion beam method, an ion plating method, a plasma polymerization method, a plasma. CVD method, a laser CVD method, a thermal CVD method, and a coating method. Of these, specifically preferred is a method employing an atmospheric pressure plasma polymerization method, described in JP-A No. 2004-68143. The “CVD method” as used herein refers to chemical vapor deposition (Chemical Vapor Deposition).

Examples of opaque support substrates include metal plates such aluminum or stainless steel films, opaque resin substrates, and ceramic substrates.

An external extraction quantum efficiency of light emitted by the organic EL element of the present invention is preferably 1.0% or more at a room temperature (25° C.), but is more preferably 5.0% or more.

The external extraction quantum efficiency mentioned here is as indicated by the following equation.

External extraction quantum efficiency (%)=(Number of photons emitted by the organic EL element to the exterior/Number of electrons fed to organic EL element)×100.

Further, it may be used simultaneously a color hue improving filter such as a color filter, or it may be used simultaneously a color conversion filter which convert emitted light color from the organic EL element to multicolor by employing fluorescent materials.

### [Sealing]

As sealing means employed in the present invention, listed may be, for example, a method in which sealing members, electrodes, and a support substrate are subjected to adhesion via adhesives. The sealing members may be arranged to cover the display region of an organic EL element, and may be a concave plate or a flat plate. Neither transparency nor electrical insulation is limited.

Examples of the sealing member include a glass plate, a polymer plate (film), and a metal plate (film). Specifically, it is possible to list, as glass plates, soda-lime glass, barium-strontium containing glass, lead glass, aluminosilicate glass, borosilicate glass, barium borosilicate glass, and quartz. Further, listed as polymer plates may be polycarbonate, acryl, polyethylene terephthalate, polyether sulfide, and polysulfone. As a metal plate, listed are those composed of at least one metal selected from the group consisting of stainless steel, iron, copper, aluminum magnesium, nickel, zinc, chromium, titanium, molybdenum, silicon, germanium, and tantalum, or alloys thereof.

In the present invention, since it is possible to achieve a thin organic EL element, it is preferable to employ a polymer film or a metal film. Further, it is preferable that the polymer film has an oxygen permeability of 1×10⁻³ mL/(m²·day·atm)or less determined by the method based on JIS K 7126-1987, and a water vapor permeability of 1×10⁻³ g/(m²·24 h) or less (at 25±0.5° C., and 90±2% RH) determined based on JIS K 7129-1992.

Conversion of the sealing member into concave is carried out by employing a sand blast process or a chemical etching process.

Examples of an adhesive for sealing are photo-curing and heat-curing types having a reactive vinyl group of acrylic acid based oligomers and methacrylic acid, as well as moisture curing types such as 2-cyanoacrylates. Further listed may be thermal and chemical curing types (mixtures of two liquids) such as epoxy based ones. Still further listed may be hot-melt type polyamides, polyesters, and polyolefins. Yet further listed may be cationic curable type UV curable epoxy resin adhesives.

In addition, since an organic EL element is occasionally deteriorated via a thermal process, preferred are those which enable adhesion and curing between a room temperature and 80° C. Further, desiccating agents may be dispersed into the aforesaid adhesives. Adhesives may be applied onto sealing portions via a commercial dispenser or printed on the same in the same manner as screen printing.

Further, it is appropriate that on the outside of the aforesaid electrode which interposes the organic layer and faces the support substrate, the aforesaid electrode and organic layer are covered, and in the form of contact with the support substrate, inorganic and organic material layers are formed as a sealing film. In this case, as materials that form the aforesaid film may be those which exhibit functions to retard penetration of moisture or oxygen which results in deterioration. For example, it is possible to employ silicon oxide, silicon dioxide, and silicon nitride.

Still further, in order to improve brittleness of the aforesaid film, it is preferable that a laminated layer structure is formed, which is composed of these inorganic layers and layers composed of organic materials. Methods to form these films are not particularly limited. It is possible to employ, for example, a vacuum deposition method, a sputtering method, a reactive sputtering method, a molecular beam epitaxy method, a cluster ion beam method, an ion plating method, a plasma polymerization method, an atmospheric pressure plasma polymerization method, a plasma. CVD method, a thermal CVD method, and a coating method.

It is possible to provide a gas phase structure and a liquid phase structure in the space between the sealing member and the display area of the organic EL element. For example, an inert gas such as nitrogen or argon or an inert liquid such as fluorinated hydrocarbon or silicone oil may be injected. It is also possible to use a vacuum in the space.

Further, it is possible to enclose hygroscopic compounds in the interior of the space.

Examples of a hygroscopic compound include: metal oxides (for example, sodium oxide, potassium oxide, calcium oxide, barium oxide, magnesium oxide, and aluminum oxide); sulfates (for example, sodium sulfate, calcium sulfate, magnesium sulfate, and cobalt sulfate); metal halides (for example, calcium chloride, magnesium chloride, cesium fluoride, tantalum fluoride, cerium bromide, magnesium bromide, barium iodide, and magnesium iodide); perchlorates (for example, barium perchlorate and magnesium perchlorate). In sulfates, metal halides, and perchlorates, suitably employed are anhydrides. For sulfate salts, metal halides and perchlorates, suitably used are anhydrous salts.

### [Protective Film and Protective Plate]

On the aforesaid sealing film which interposes the organic layer and faces the support substrate or on the outside of the aforesaid sealing film, a protective or a protective plate may be arranged to enhance the mechanical strength of the element. Specifically, when sealing is achieved via the aforesaid sealing film, the resulting mechanical strength is not always high enough, therefore it is preferable to arrange the protective film or the protective plate described above.

Usable materials for these include glass plates, polymer plate-films, and metal plate-films which are similar to those employed for the aforesaid sealing. However, from the viewpoint of reducing weight and thickness, it is preferable to employ a polymer film.

### [Improving Method of Light Extraction]

It is generally known that an organic EL element emits light in the interior of the light emitting layer exhibiting the refractive index (refractive index: 1.6 to 2.1) which is greater than that of air, whereby only about 15% to 20% of light generated in the light emitting layer is extracted. This is due to the fact that light incident to an interface (an interlace of a transparent substrate to air) at an angle of θ which is at least critical angle is not extracted to the exterior of the element due to the resulting total reflection, or light is totally reflected between the transparent electrode or the light emitting layer and the transparent substrate, and light is guided via the transparent electrode or the light emitting layer, whereby light escapes in the direction of the organic EL element side surface.

Means to enhance the efficiency of the aforesaid light extraction include, for example: a method in which roughness is formed on a surface of a transparent substrate, whereby total reflection is minimized at the interface of the transparent substrate to air (U.S. Pat. No. 4,774,435), a method in which efficiency is enhanced in such a manner that a substrate results in light collection (JP-A 63-314795), a method in which a reflection surface is formed on the side of the element (JP-A 1-220394), a method in which a flat layer of a middle refractive index is introduced between the substrate and the light emitting body and an antireflection film is formed (JP-A 62-172691), a method in which a flat layer of a refractive index which is equal to or less than the substrate is introduced between the substrate and the light emitting body (JP-A 2001-202827), and a method in which a diffraction grating is formed between the substrate and any of the layers such as the transparent electrode layer or the light emitting layer (including between the substrate and the outside) (JP-A 11-283751).

In the present invention, it is possible to employ these methods while combined with the organic EL element of the present invention. Of these, it is possible to appropriately employ the method in which a flat layer of a refractive index which is equal to or less than the substrate is introduced between the substrate and the light emitting body and the method in which a diffraction grating is formed between any layers of a substrate, and a transparent electrode layer and a light emitting layer (including between the substrate and the outside space).

In the present invention, by combining these means, it is possible to obtain an organic EL element having higher luminance and excellent durability.

When a low refractive index medium having a thickness of greater than the wavelength of light is formed between the transparent electrode and the transparent substrate, the extraction efficiency of light emitted from the transparent electrode to the exterior increases as the refractive index of the medium decreases.

As materials of the low refractive index layer, listed are, for example, aerogel, porous silica, magnesium fluoride, and fluorine based polymers. Since the refractive index of the transparent substrate is commonly about 1.5 to 1.7, the refractive index of the low refractive index layer is preferably approximately 1.5 or less. More preferably, it is 1.35 or less.

Further, thickness of the low refractive index medium is preferably at least two times of the wavelength in the medium. The reason is that, when the thickness of the low refractive index medium reaches nearly the wavelength of light so that electromagnetic waves escaped via evanescent enter into the substrate, effects of the low refractive index layer are lowered.

The method in which the interface which results in total reflection or a diffraction grating is introduced in any of the media is characterized in that light extraction efficiency is significantly enhanced. The above method works as follows. By utilizing properties of the diffraction grating capable of changing the light direction to the specific direction different from diffraction via so-called Bragg diffraction such as primary diffraction or secondary diffraction of the diffraction grating, of light emitted from the light entitling layer, light, which is not emitted to the exterior due to total reflection between layers, is diffracted via introduction of a diffraction grating between any layers or in a medium (in the transparent substrate and the transparent electrode) so that light is extracted to the exterior.

It is preferable that the introduced diffraction grating exhibits a two-dimensional periodic refractive index. The reason is as follows. Since light emitted in the light emitting layer is randomly generated to all directions, in a common one-dimensional diffraction grating exhibiting a periodic refractive index distribution only in a certain direction, light which travels to the specific direction is only diffracted, whereby light extraction efficiency is not sufficiently enhanced

However, by changing the refractive index distribution to a two-dimensional one, light traveling in all directions is diffracted, and the light extraction efficiency is increased.

A position to introduce a diffraction grating may be between any layers or in a medium (in a transparent substrate or a transparent electrode). However, a position near the organic light emitting layer, where light is generated, is preferable. In this case, the cycle of the diffraction grating is preferably from about ½ to 3 times of the wavelength of light in the medium. The preferable arrangement of the diffraction grating is such that the arrangement is two-dimensionally repeated in the form of a square lattice, a triangular lattice, or a honeycomb lattice.

### [Light Collection Sheet]

In the organic EL element of the present invention, via a process to arrange a structure such as a micro-lens array shape on the light extraction side of the support substrate (substrate) or via combination with a so-called light collection sheet, light is collected in the specific direction such as the front direction with respect to the light emitting element surface of the organic EL element, whereby it is possible to enhance luminance in the specific direction.

In an example of the micro-lens array, square pyramids to realize a side length of 30 μm and an apex angle of 90 degrees are two-dimensionally arranged on the light extraction side of the substrate. The side length is preferably 10 to 100 μm. When it is less than the lower limit, coloration occurs due to generation of diffraction effects, while when it exceeds the upper limit, the thickness increases undesirably.

It is possible to employ, as a light collection sheet, for example, one which is put into practical use in the LED backlight of liquid crystal display devices. It is possible to employ, as such a sheet, for example, the luminance enhancing film (BEF), produced by Sumitomo 3M Limited. As shapes of a prism sheet employed may be, for example, A shaped stripes of an apex angle of 90 degrees and a pitch of 50 μm formed on a substrate, a shape in which the apex angle is rounded, a shape in which the pitch is randomly changed, and other shapes.

Further, in order to control the light radiation angle from the light emitting element, simultaneously employed may be a light diffusion plate-film. For example, it is possible to employ the diffusion film (LIGHT-UP), produced by Kimoto Co., Ltd.

### [Applications]

The organic EL element of the present invention may be used as an electronic device, for example, a display device, a display, and various light emitting devices.

Examples of the light emitting device include: lighting apparatuses (for example, home lighting and car lighting), backlights for clocks and liquid crystals, sign advertisements, signals, light sources of light memory media, light sources of electrophotographic copiers, light sources of light communication processors, and light sources of light sensors. The present invention is not limited to them. It is especially effectively employed as a backlight of a liquid crystal display device and a lighting source.

As needed, the organic EL element of the present, invention may undergo patterning via a metal mask or an ink-jet printing method during film formation. When the patterning is carried out, only an electrode may undergo patterning, an electrode and a light emitting layer may undergo patterning, or all of the organic EL element layers may undergo patterning. During preparation of the organic EL element, it is possible to employ conventional methods.

### <<Display Device>>

A display device provided with an organic EL element of the present invention may emit a single color or multiple colors. Here, it will be described a multiple color display device.

In case of a multiple color display device, a shadow mask is placed during the formation of a light emitting layer, and a layer is formed as a whole with a vapor deposition method, a cast method, a spin coating method, an inkjet method, and a printing method.

When patterning is done only to the light emitting layer, although the coating method is not limited in particular, preferable methods are a vapor deposition method, an inkjet method, a spin coating method, and a printing method.

A configuration of an organic EL element provided for a display device is selected from the above-described examples of an organic EL element according to the necessity.

The production method of an organic EL element is described as an embodiment of a production method of the above-described organic EL element.

When a direct-current voltage is applied to the produced multiple color display device, light emission may be observed by applying voltage of 2 o 40 V by setting the anode to have a plus (+) polarity, and the cathode to have a minus (−) polarity. When the voltage is applied to the device with reverse polarities, an electric current does not pass and light emission does not occur. Further, when an alternating-current voltage is applied to the device, light emission occurs only when the anode has a plus (+) polarity and the cathode has a minus (−) polarity. In addition, an arbitrary wave shape may be used for applying alternating-current.

The multiple color display device may be used for a display device, a display, and a variety of light emitting sources. In a display device or a display, a full color display is possible by using 3 kinds of organic EL elements emitting blue, red and green.

Examples of a display device or a display are: a television set, a personal computer, a mobile device, an AV device, a character broadcast display, and an information display in a car. Specifically, it may be used for a display device reproducing a still image or a moving image. When it is used for a display device reproducing a moving image, the driving mode may be any one of a simple matrix (a passive-matrix) mode and an active-matrix mode.

Examples of a light emitting device include: home lighting, car lighting, backlights for clocks and liquid crystals, sign advertisements, signals, light sources of light memory media, light sources of electrophotographic copiers, light sources of light communication processors, and light sources of light sensors. The present invention is not limited to them.

In the following, an example of a display device provided with an organic EL element of the present invention will be described by referring to drawings.

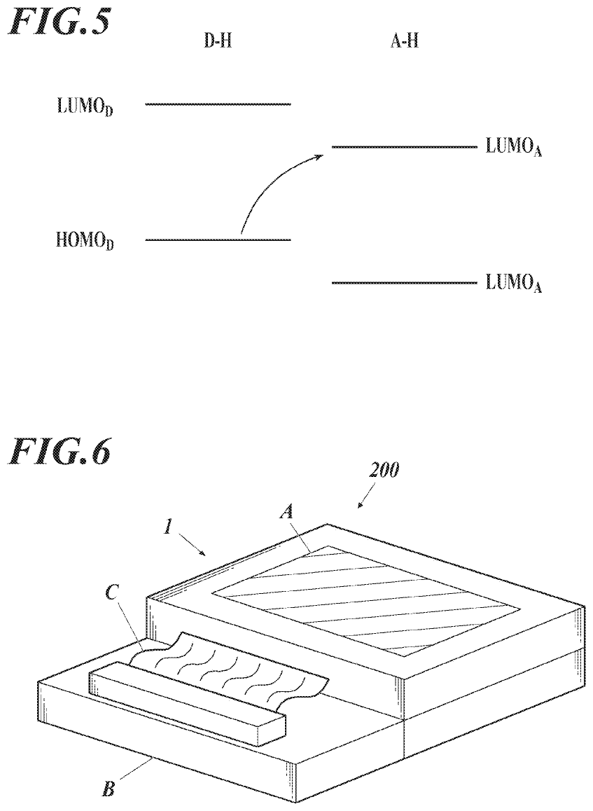

FIG. 6 is a schematic perspective view illustrating an example of a display device composed of an organic EL element. Display of image information is carried out by light emission of an organic EL element. For example, it is a schematic drawing of a display of a cell-phone.

In a display device (200) indicated in FIG. 6, a display (1) is constituted of a display section (A) having plural number of pixels, a control section (B) which performs image scanning of the display section (A) based on image information, and a wiring section (C) electrically connecting the display section (A) and the control section (B).

The control section (B), which is electrically connected to the display section (A) via the wiring section (C), sends a scanning signal and an image data signal to plural number of pixels based on image information from the outside and pixels of each scanning line successively emit depending on the image data signal by a scanning signal to perform image scanning, whereby image information is displayed on the display section (A).

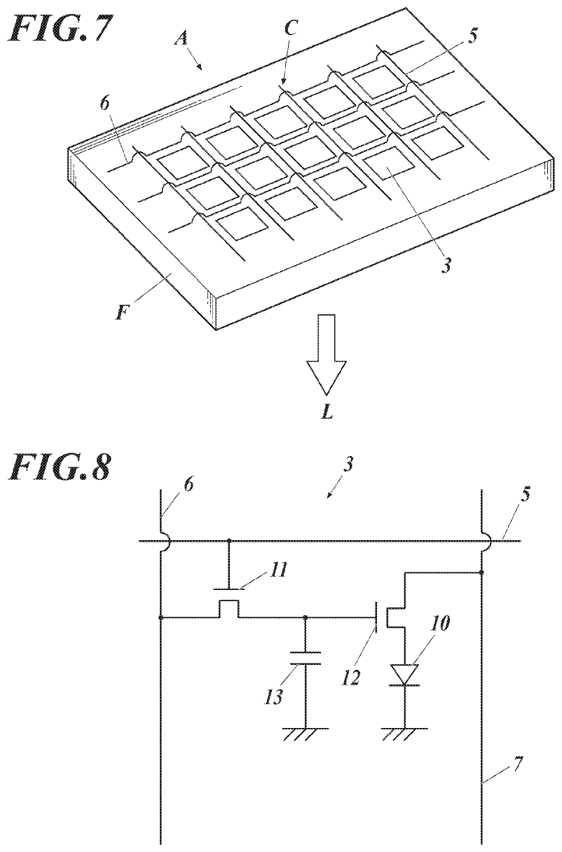

FIG. 7 is a schematic drawing illustrating an example of the configuration of a display device by the active matrix method.

In FIG. 7, the display section (A) is provided with the wiring section (C), which contains plural scanning lines (5) and data lines (6), and plural pixels (3) on a substrate (F).

FIG. 7 illustrates a case where the emitted light (L) emitted in the pixel (3) is extracted in the direction of the white arrow (downward) which is the substrate (F) side.

The scanning lines (5) and the plural data lines (6) in the wiring section (C) each are composed of a conductive material, and the scanning lines (5) and the data lines (6) are perpendicular in a grid form and are connected to pixels (3) at the right-angled crossing points (in FIG. 7, a detailed configuration such as the connection method is not shown).

The pixel (3) receives an image data from the data line (6) when a scanning signal is applied from the scanning line (5) and emits light according to the received image data.

Full-color image display is possible by appropriately arranging pixels having an emission color in a red region, pixels in a green region and pixels in a blue region, side by side on the same substrate.

Next, an emission process of a pixel will be explained.

FIG. 8 is a schematic wiring diagram showing an example of a circuit of a light emitting pixel.

Each pixel (3) is equipped with an organic EL element (10), a switching transistor (11), an operating transistor (12) and a capacitor (13). Red (R), green (G) and blue (B) emitting organic EL elements (10) are utilized as the organic EL element (10) for plural pixels (3), and full-color display device is possible by arranging these side by side on the same substrate.

In FIG. 8, an image data signal is applied on the drain of the switching transistor (11) via the data line (6) from the control section (not illustrated). Then, when a scanning signal is applied on the gate of the switching transistor (11) via the scanning line (5) from the control section (not illustrated), operation of switching transistor (11) is on to transmit the image data signal applied on the drain to the gates of the capacitor (13) and the operating transistor (12).

The operating transistor (12) is on, simultaneously with the capacitor (13) being charged depending on the potential of an image data signal, by transmission of an image data signal. In the operating transistor (12), the drain is connected to an electric source line (7) and the source is connected to the electrode of the organic EL element (10), and an electric current is supplied from the electric source line (7) to the organic EL element (10) depending on the potential of an image data applied on the gate.

When a scanning signal is transferred to the next scanning line (5) by successive scanning of the control section (not illustrated), operation of the switching transistor (11) is off. However, since the capacitor (13) keeps the charged potential of an image data signal even when operation of the switching transistor (11) is off, operation of the operating transistor (12) is kept on to continue emission of the organic EL element (10) until the next scanning signal is applied. When the next scanning signal is applied by successive scanning, the operating transistor (12) operates depending on the potential of an image data signal synchronized to the scanning signal and the organic EL element (10) emits light.

That is, light emission of each organic EL element (10) of the plural pixels (3) is performed by providing the switching transistor (11) and the operating transistor (12) against each organic EL element (10) of plural pixels (3). Such a light emission method is called as an active matrix mode.

Herein, emission of the organic EL element (10) may be either emission of plural gradations based on a multiple-valued image data signal having plural number of gradation potentials or on and off of a predetermined emission quantity based on a binary image data signal. Further, potential hold of the capacitor (13) may be either continuously maintained until the next scanning signal application or discharged immediately before the next scanning signal application.

In the present invention, emission operation is not necessarily limited to the above-described active matrix mode, but may be a passive matrix mode in which organic EL element (10) is emitted based on a data signal only when a scanning signal is scanned.

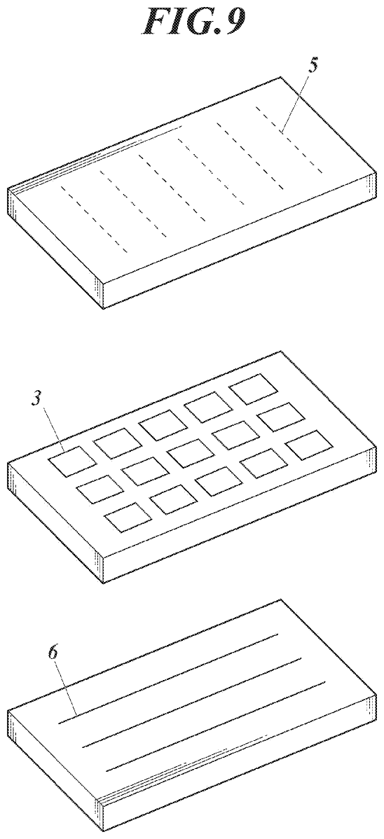

FIG. 9 is schematic perspective view illustrating an example of the configuration of a display device based on a passive matrix method.

In FIG. 9, a plurality of scanning lines (5) and a plurality of image data lines (6) are arranged grid-wise, opposing to each other and sandwiching the pixels (3).

When a scanning signal of the scanning line (5) is applied by successive scanning, the pixel (3) connected to the scanning line (5) applied with the signal emits light depending on an image data signal.

Since the pixel (3) is provided with no active element in a passive matrix mode, decrease of manufacturing cost is possible.

By employing the organic EL element of the present invention, it was possible to obtain a display device having improved luminous efficiency.

### <<Light Emitting Device>>

An organic EL element of the present invention may be used for a light emitting device.

An organic EL element of the present invention may be provided with a resonator structure. The intended uses of the organic EL element provided with a resonator structure are: a light source of a light memory media, a light source of an electrophotographic copier, a light source of a light communication processor, and a light source of a light sensor, however, it is not limited to them. It may be used for the above-described purposes by making to emit a laser.

Further, an organic EL element of the present invention may be used for a kind of lamp such as for illumination or exposure. It may be used for a projection device for projecting an image, or may be used for a display device (display) to directly observe a still image or a moving image thereon.

The driving mode used for a display device of a moving image reproduction may be any one of a passive matrix mode and an active matrix mode. By employing two or more kinds of organic EL elements of the present invention emitting a different emission color, it may produce a full color display device.

In addition, the compound of the present invention may be applied to a lighting device equipped with an organic EL element that produces substantially white light emission. For example, when a plurality of light emitting materials are employed, white light may be obtained by mixing colors of a plurality of emission colors. As a combination of the plurality of emission colors, it may be a combination of red, green and blue having emission maximum wavelength of three primary colors, or it may be a combination of colors having two emission maximum wavelength making use of the relationship of two complementary colors of blue and yellow, or blue-green and orange.

A production method of an organic EL element of the present invention is done by placing a mask only during formation of a light emitting layer, a hole transport layer and an electron transport layer. It may be produced by coating with a mask to make simple arrangement. Since other layers are common, there is no need of pattering with a mask For example, it may produce an electrode uniformly with a vapor deposition method, a cast method, a spin coating method, an inkjet method, and a printing method. The production yield will be improved.

According to this method, unlike the white organic EL device in which light emitting elements of a plurality of colors are arranged in parallel in an array, the elements themselves emit white light.

### One Embodiment of Lighting Device of the Present Invention

One embodiment of lighting devices of the present invention provided with an organic EL element of the present invention will be described.

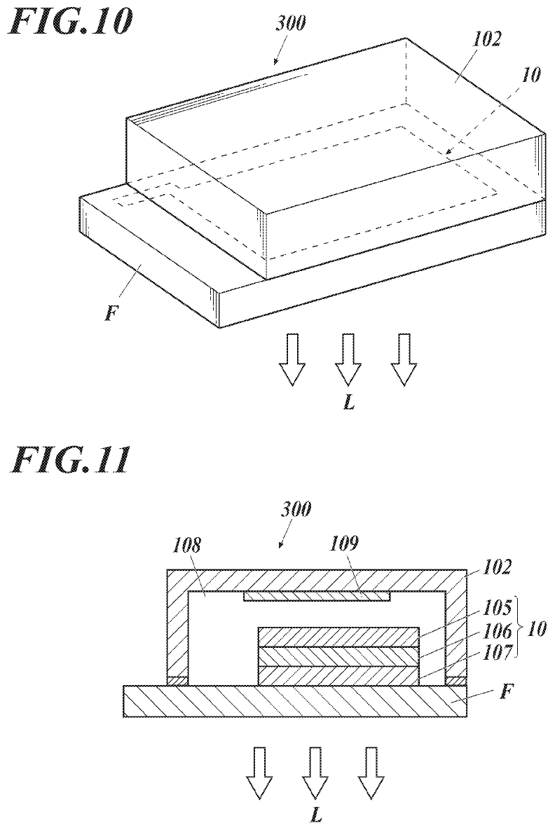

The non-light emitting surface of the organic EL element of the present invention was covered with a glass case, and a 300 μm thick glass substrate was employed as a sealing substrate. An epoxy based light curable type adhesive (LUXTRACK LC0629B produced by Toagosei Co., Ltd.) was employed in the periphery as a sealing material. The resulting one was superimposed on the aforesaid cathode to be brought into close contact with the aforesaid transparent support substrate, and curing and sealing were carried out via exposure of UV radiation onto the glass substrate side, whereby the lighting device (300) illustrated in FIG. 10 and FIG. 11, was formed.

FIG. 10 is a schematic perspective view of a lighting device (300). An organic EL element of the present invention (organic EL element (10) in a light emitting device) formed on a substrate (F) is covered with glass cover (102). Incidentally, sealing by the glass cover was carried out in a globe box under nitrogen ambience (under an ambience of high purity nitrogen gas at a purity of at least 99.999%) so that organic EL Element (10) was not brought into contact with atmosphere).

FIG. 11 is a cross-sectional view of a lighting device (300). In FIG. 11, F represents a substrate, 105 represents a cathode, 106 represents an organic functional layer group, and 107 represents a glass substrate fitted with a transparent electrode (anode). Further, the interior of glass cover (102) is filled with nitrogen gas (108) and water catching agent (109) is provided.

By employing an organic EL element of the present invention, it was possible to obtain a lighting device having improved luminous efficiency.

### EXAMPLES

Hereafter, the present invention will be described specifically by referring to Examples, however, the present invention is not limited to them. The indication of “%” is used in Examples. Unless particularly mentioned, it represents “mass %”.

### <<Compounds Used in Examples>>

First, the method for synthesizing the compounds used in the examples shown below and their structures will be described.

### [Synthesis of Compound Having Structure Represented by Formula (1)]

### (Ethylene Linker Type Compound: Synthesis of Exemplified Compound E-1)

In accordance with the description in Non-patent document N. K. Garg, et al., J. Am. Chem. Soc., 2004, 126, 9552-9553, 3-ethynyl-9-phenylcarbazole, 2-(4-bromophenyl)-4,6-diphenyl-1,3,5-triazine, Pd/C (Pd 10%) made by Tokyo Chemical Industry co., Ltd, and tetrahydrofuran were mixed. The reaction mixture was stirred under hydrogen atmosphere (1 atm) at room temperature for 3 hours to obtain a crude product of Exemplified compound E-1. Thereafter, column chromatography, recrystallization and sublimation purification were performed to obtain a high purity product of an exemplified compound E-1.

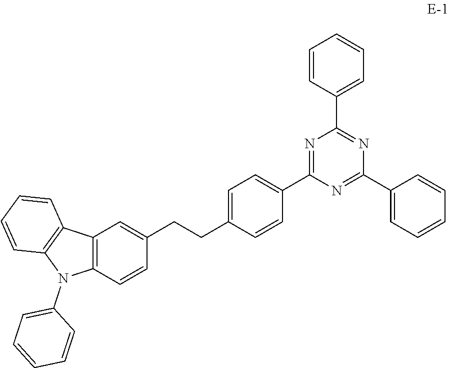 [SMILES: c1ccc(-c2nc(-c3ccccc3)nc(-c3ccc(CCc4ccc5c(c4)c4ccccc4n5-c4ccccc4)cc3)n2)cc1]

### (Other Ethylene Linker Type Compounds)

The following ethylene linker type compounds were synthesized in the same manner as the method of synthesizing the above-mentioned exemplified compound E-1.

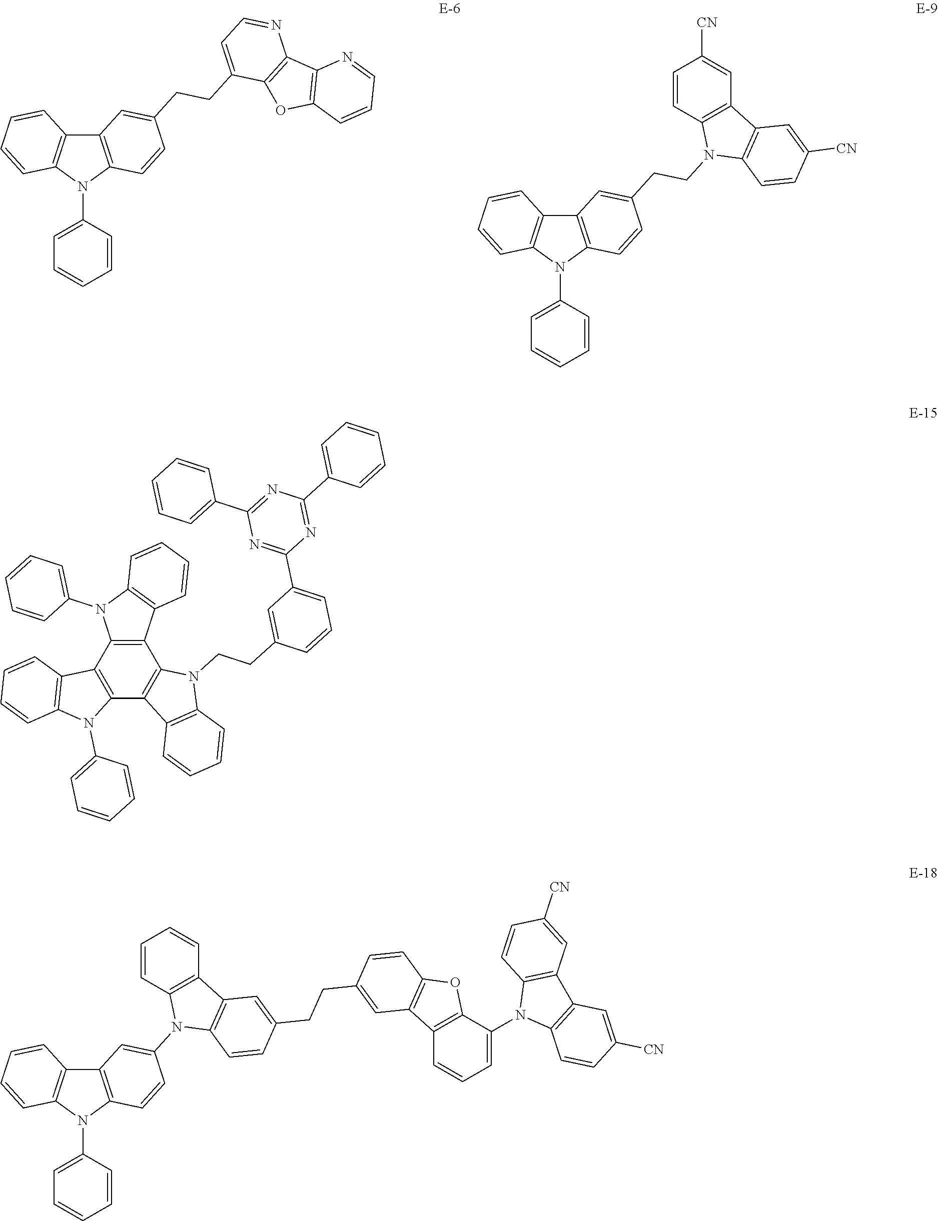 [SMILES: c1ccc(-n2c3ccccc3c3cc(CCc4ccnc5c4oc4cccnc45)ccc32)cc1 ; N#Cc1ccc2c(c1)c1cc(C#N)ccc1n2CCc1ccc2c(c1)c1ccccc1n2-c1ccccc1 ; c1ccc(-c2nc(-c3ccccc3)nc(-c3cccc(CCn4c5ccccc5c5c4c4c6ccccc6n(-c6ccccc6)c4c4c6ccccc6n(-c6ccccc6)c54)c3)n2)cc1 ; N#Cc1ccc2c(c1)c1cc(C#N)ccc1n2-c1cccc2c1oc1ccc(CCc3ccc4c(c3)c3ccccc3n4-c3ccc4c(c3)c3ccccc3n4-c3ccccc3)cc12]

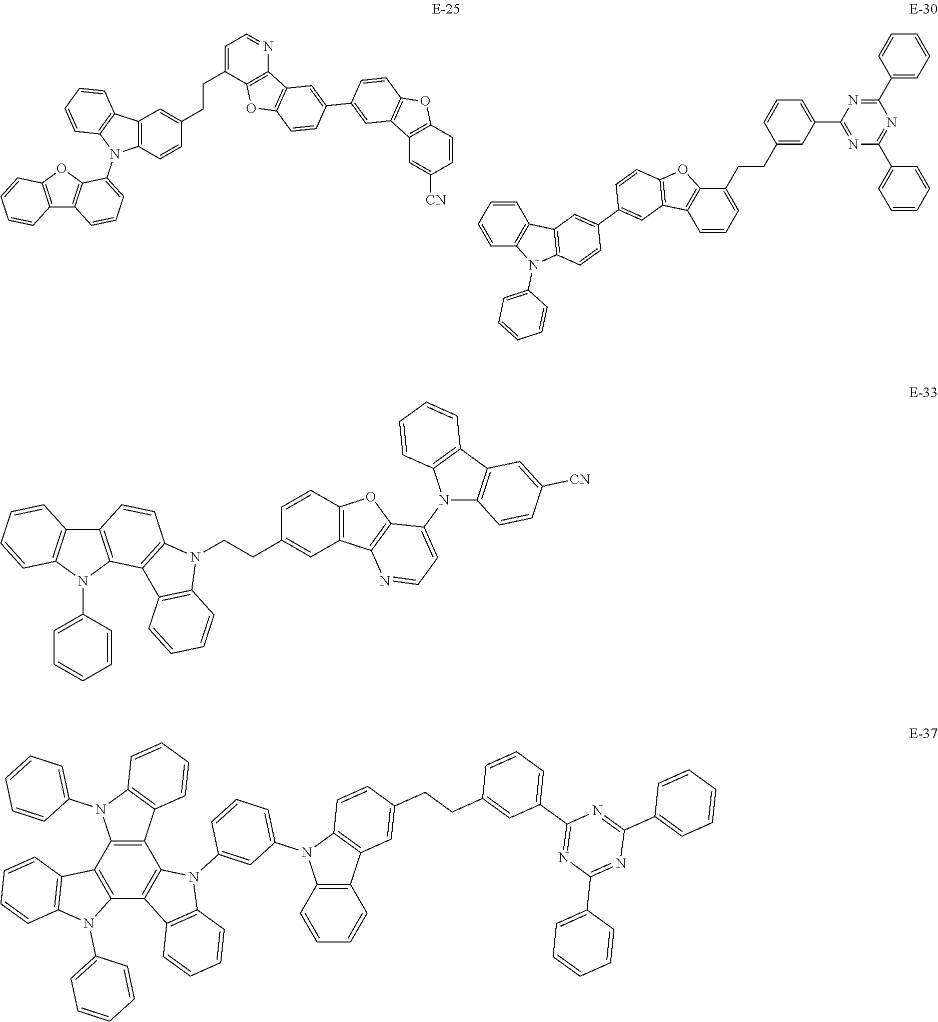 [SMILES: N#Cc1ccc2oc3ccc(-c4ccc5oc6c(CCc7ccc8c(c7)c7ccccc7n8-c7cccc8c7oc7ccccc78)ccnc6c5c4)cc3c2c1 ; c1ccc(-c2nc(-c3ccccc3)nc(-c3cccc(CCc4cccc5c4oc4ccc(-c6ccc7c(c6)c6ccccc6n7-c6ccccc6)cc45)c3)n2)cc1 ; N#Cc1ccc2c(c1)c1ccccc1n2-c1ccnc2c1oc1ccc(CCn3c4ccccc4c4c3ccc3c5ccccc5n(-c5ccccc5)c34)cc12 ; c1ccc(-c2nc(-c3ccccc3)nc(-c3cccc(CCc4ccc5c(c4)c4ccccc4n5-c4cccc(-n5c6ccccc6c6c7c(c8ccccc8n7-c7ccccc7)c7c(c8ccccc8n7-c7ccccc7)c65)c4)c3)n2)cc1]

### (Cyclohexyl Linker Type Compound: Synthesis of Exemplified Compound E-77)

In accordance with the description in Non-patent document M. Linseis, et al., J. Am. Chem. Soc., 2012, 134, 16671-16692, 1,2-bis-(4-bromophenyl)-cyclohex-1-ene was obtained. Next, 1,2-bis-(4-bromophenyl)-cyclohex-1-ene, (1,5-cyclooctadiene)(pyridine)(tricyclohexylphosphine)-iridium (I) hexafluorophosphate made by Sigma Aldrich, and dichloroethane were mixed. The reaction mixture was stirred under hydrogen atmosphere (1 atm) at room temperature for 8 hours to obtain 1,2-bis-(4-bromophenyl)-cyclohexane. Next, 1,2-bis-(4-bromophenyl)-cyclohexane, 9H-carbazole manufactured by Tokyo Chemical Industry Co., Ltd., palladium (II) acetate, tri-t-butyl phosphine, sodium-t-butoxide, and o-xylene were mixed. The mixture was mixed and heated at 130° C. with stirring for 6 hours to obtain an exemplified compound E-77 precursor P.

Next, the Exemplified compound E-77 precursor P, 9H-carbazole-3,6-dicarbonitrile, palladium (II) acetate manufactured by Tokyo Chemical Industry Co., Ltd., tri-t-butylphosphine, sodium-t-butoxide, and o-xylene were mixed. The mixture was mixed and heated at 130° C. with stirring for 6 hours to obtain a crude product of Exemplified compound E-77. Thereafter, column chromatography, recrystallization and sublimation purification were performed to obtain a high purity product of an exemplified compound E-77.

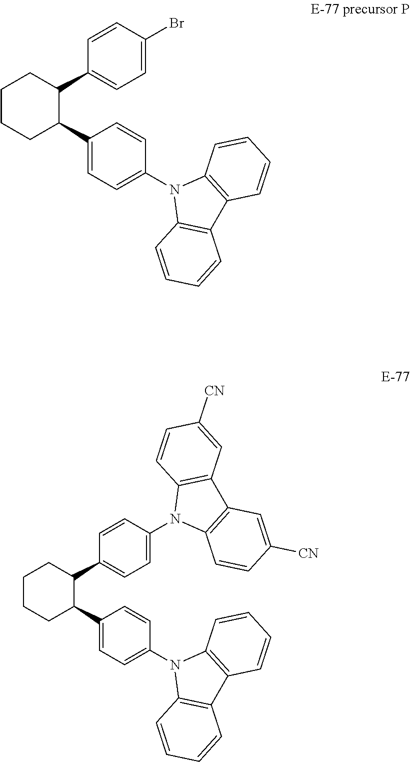

### (Other Cyclohexyl Linker Type Compounds)

The following cyclohexyl linker type compounds were synthesized in the same manner as the method of synthesizing the above-mentioned exemplified compound E-78.

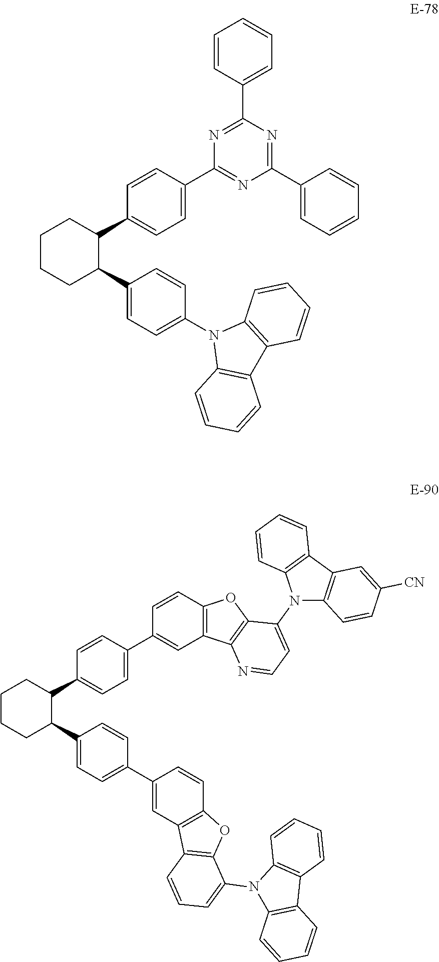

The HOMO and LUMO values of the substituents corresponding to D-H and A-H in the compound having a structure represented by Formula (1) used in the examples are indicated in Table I.

TABLE I

| | HOMO | | LUMO | |
|---|---|---|---|---|
| | D-H | A-H | D-H | A-H |
| Exemplified | −5.33 | −6.65 | −0.65 | −1.80 |
| compound 1 | | | | |
| Exemplified | −5.33 | −6.57 | −0.65 | −1.62 |
| compound 6 | | | | |
| Exemplified | −5.33 | −6.45 | −0.65 | −1.71 |
| compound 9 | | | | |
| Exemplified | −4.84 | −6.65 | −0.62 | −1.80 |
| compound 15 | | | | |
| Exemplified | −5.13 | −6.22 | −0.95 | −1.68 |
| compound 18 | | | | |
| Exemplified | −5.33 | −6.13 | −1.18 | −1.51 |
| compound 25 | | | | |
| Exemplified | −5.22 | −6.65 | −0.89 | −1.80 |
| compound 30 | | | | |
| Exemplified | −5.03 | −6.01 | −0.61 | −1.78 |
| compound 33 | | | | |
| Exemplified | −4.88 | −6.65 | −0.74 | −1.80 |
| compound 37 | | | | |
| Exemplified | −5.33 | −6.25 | −0.65 | −1.62 |
| compound 77 | | | | |
| Exemplified | −5.33 | −6.65 | −0.65 | −1.80 |
| compound 78 | | | | |
| Exemplified | −5.34 | −6.01 | −1.21 | −1.79 |
| compound 90 | | | | |

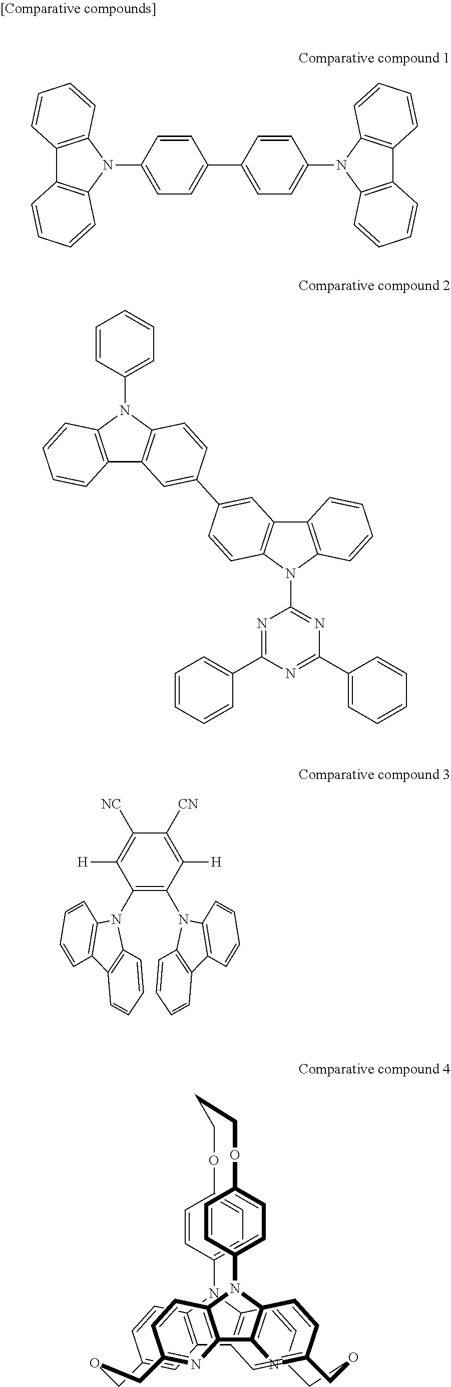

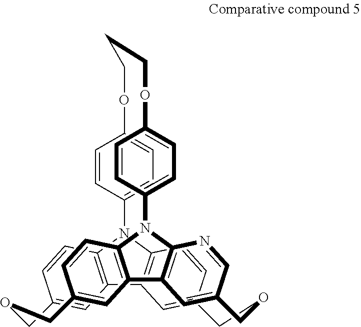

### [Material for Forming Organic Functional Layer of Organic EL Element]

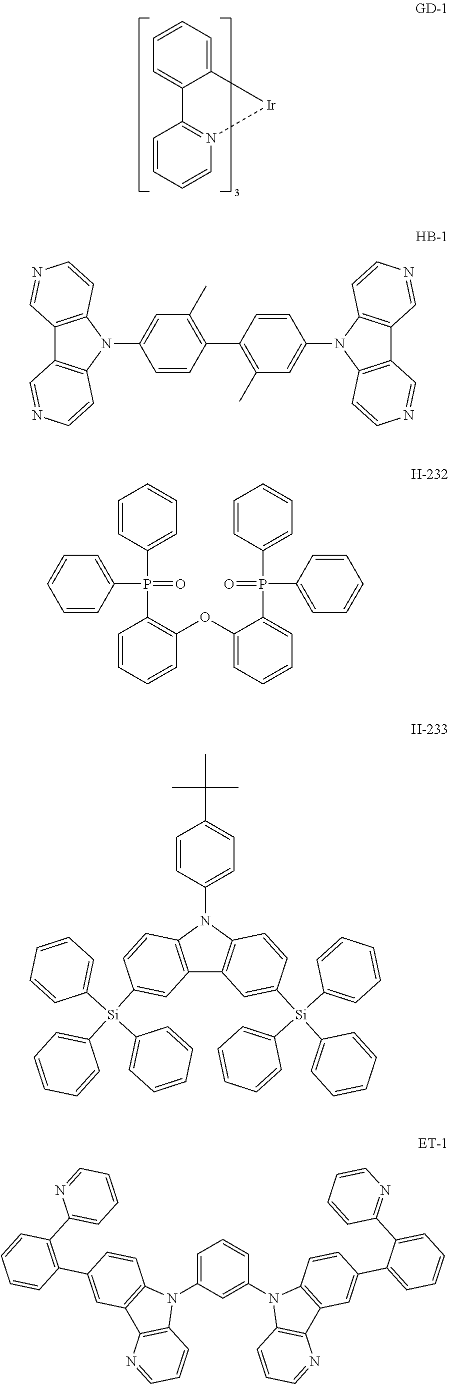

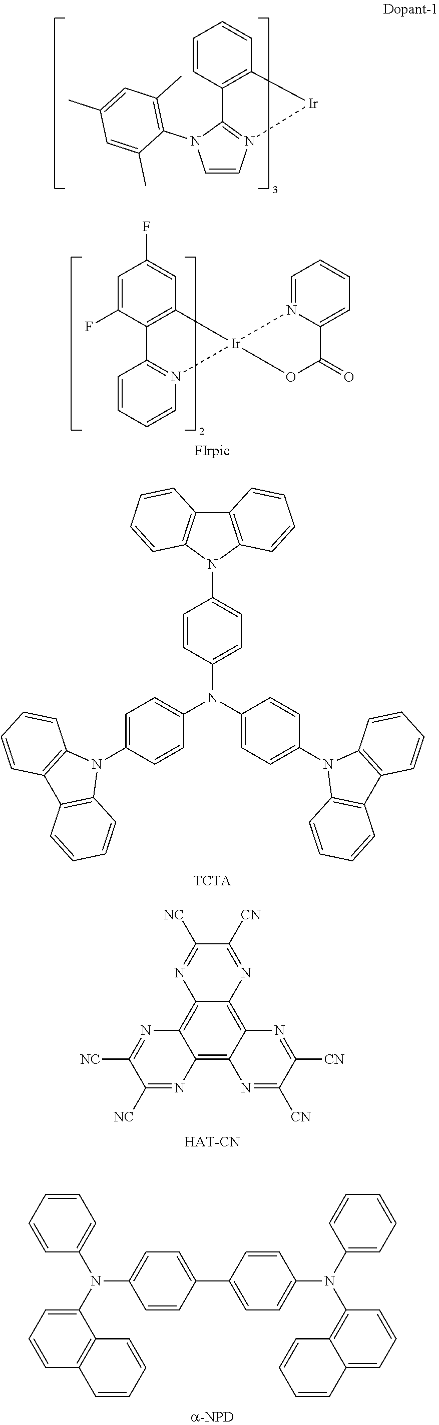

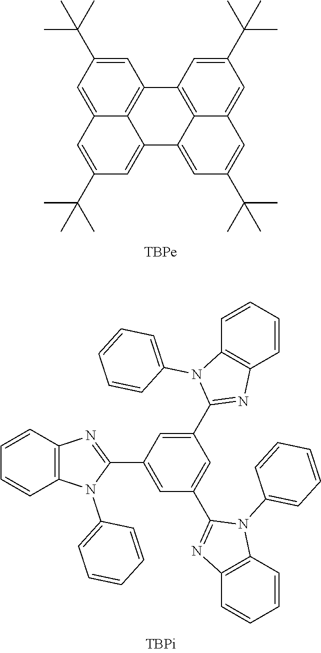

### Example 1

### [Preparation of Organic EL Element]

### (Preparation of Organic EL Element 1-1)

An ITO transparent electrode (anode) was prepared on a transparent substrate made of a glass substrate of 50 mm×50 mm with a thickness of 0.7 mm by forming a film of ITO (indium tin oxide) with a thickness of 150 nm using a commercially available vacuum deposition apparatus, then by making patterning to it. The transparent substrate provided with the ITO transparent electrode was subjected to ultrasonic washing with isopropyl alcohol, followed by drying with desiccated nitrogen gas, and it was subjected to UV ozone washing for 5 minutes. Thereafter, the transparent substrate was fixed to a substrate holder of a commercial vacuum deposition apparatus.

The constituting materials for each layer were loaded in each resistance heating boat for vapor deposition in the vacuum deposition apparatus with an optimum amount. As a resistance heating boat for vapor deposition, a resistance heating boat made of molybdenum or tungsten was used.

After reducing the pressure of a vacuum tank to 4×10⁻⁴ Pa, the resistance heating boat containing HAT-CN (1,4,5,8,9,12-hexaazatriphenylene hexacarbonitrile) was heated via application of electric current and vapor deposition was made onto the ITO transparent electrode at a deposition rate of 0.1 nm/second, whereby a hole injection layer having a thickness of 10 nm was produced.

Then, α-NPD (4,4′-bis[N-(naphthyl)-N-phenylamino]biphenyl) was deposited onto the hole injection layer at a deposition rate of 0.1 nm/second, whereby a hole transport layer having a thickness of 40 nm was produced.

Then, the resistance heating boat containing a comparative compound 1 as a host compound, and the resistance heating boat containing GD-1 were heated via application of electric current and vapor co-deposition was made onto the hole transport layer at a deposition rate of 0.1 nm/second, and 0.010 nm/second respectively, whereby a light emitting layer having a thickness of 40 nm was produced.

Then, HB-1 was vapor deposited on the light emitting layer at a vapor deposition rate of 0.1 nm/sec to form a first electron transport layer having a thickness of 5 nm.

Further, ET-1 was vapor deposited at a deposition rate of 0.1 nm/sec on the first electron transport layer to form a second electron transport layer having a thickness of 45 nm.

Further, after forming a lithium fluoride layer having a thickness of 0.5 nm on the second electron transport layer, 100 nm thick aluminum was vapor deposited to form a cathode. Thus, an organic EL element 1-1 was prepared.

### (Preparation of Organic EL Elements 1-2 to 1-23)

Organic EL elements 1-2 to 1-23 were prepared in the same manner as preparation of the organic EL element 1-1 except that the light emitting compound and the host compound used in the formation of the light emitting layer were changed to the combination of the light emitting compound and the host compound as described in Table II.

### [Evaluation of Organic El Element]

### (Measurement of Relative Luminous Efficiency)

Each organic EL element thus produced was allowed to emit light by applying a constant electric current of 2.5 mA/cm² at room temperature (about 25° C.). The light emission luminance immediately after starting to emit light was measured with Spectroradiometer CS-2000 (produced by Konica Minolta, Inc.).

Then, the light emission luminance of the obtained organic EL element 1-1 was set to 100%, the relative light emission luminance of each organic EL element was determined, and this was regarded as the relative luminous efficiency. The results were indicated in Table II. The larger the value, the better the luminous efficiency.

TABLE II

| | | | Relative | |
|---|---|---|---|---|
| Organic | Light | | luminous | |
| EL | emitting | | efficiency | |
| element | compound | Host compound | (%) | Remarks |
| 1-1 | GD-1 | Comparative | 100 | Comparative |
| | | compound 1 | | example |
| 1-2 | GD-1 | Comparative | 95 | Comparative |
| | | compound 2 | | example |
| 1-3 | GD-1 | Comparative | 80 | Comparative |
| | | compound 3 | | example |
| 1-4 | GD-1 | Comparative | 70 | Comparative |
| | | compound 4 | | example |
| 1-5 | GD-1 | Comparative | 75 | Comparative |
| | | compound 5 | | example |
| 1-6 | GD-1 | Exemplified | 130 | Present |
| | | compound E-1 | | invention |
| 1-7 | GD-1 | Exemplified | 153 | Present |
| | | compound E-6 | | invention |
| 1-8 | GD-1 | Exemplified | 165 | Present |
| | | compound E-9 | | invention |
| 1-9 | GD-1 | Exemplified | 148 | Present |
| | | compound E-15 | | invention |
| 1-10 | GD-1 | Exemplified | 170 | Present |
| | | compound E-18 | | invention |
| 1-11 | GD-1 | Exemplified | 172 | Present |
| | | compound E-25 | | invention |
| 1-12 | GD-1 | Exemplified | 151 | Present |
| | | compound E-30 | | invention |
| 1-13 | GD-1 | Exemplified | 152 | Present |
| | | compound E-33 | | invention |
| 1-14 | GD-1 | Exemplified | 160 | Present |
| | | compound E-37 | | invention |
| 1-15 | GD-1 | Exemplified | 146 | Present |
| | | compound E-77 | | invention |
| 1-16 | GD-1 | Exemplified | 142 | Present |
| | | compound E-78 | | invention |
| 1-17 | GD-1 | Exemplified | 156 | Present |
| | | compound E-90 | | invention |
| 1-18 | FIrPic | Exemplified | 137 | Present |
| | | compound E-15 | | invention |
| 1-19 | FIrPic | Exemplified | 142 | Present |
| | | compound E-18 | | invention |
| 1-20 | FIrPic | Exemplified | 129 | Present |
| | | compound E-77 | | invention |
| 1-21 | Dopant-1 | Exemplified | 147 | Present |
| | | compound E-15 | | invention |
| 1-22 | Dopant-1 | Exemplified | 135 | Present |
| | | compound E-18 | | invention |
| 1-23 | Dopant-1 | Exemplified | 138 | Present |
| | | compound E-77 | | invention |

As is clear from the results described in Table II, the organic EL element of the present invention using the compound of the present invention having a structure represented by Formula (1) as a host compound is excellent in luminous efficiency than the comparative example.

### Example 2

### [Preparation of Organic EL Element]

### (Preparation of Organic EL Element 2-1)

An ITO transparent electrode (anode) was prepared on a glass substrate of 50 mm×50 mm with a thickness of 0.7 mm by forming a film of ITO (indium tin oxide) with a thickness of 150 nm using a commercially available vacuum deposition apparatus, then by making patterning to it. The transparent substrate provided with the ITO transparent electrode was subjected to ultrasonic washing with isopropyl alcohol, followed by drying with desiccated nitrogen gas, and it was subjected to UV ozone washing for 5 minutes. Thereafter, the transparent substrate was fixed to a substrate holder of a commercial vacuum deposition apparatus.

The constituting materials for each layer were loaded in each resistance heating boat for vapor deposition in the vacuum deposition apparatus with an optimum amount. As a resistance heating boat for vapor deposition, a resistance heating boat made of molybdenum or tungsten was used.

After reducing the pressure of a vacuum tank to 4×10⁻⁴ Pa, the resistance heating boat containing α-NPD (4,4′-bis[N-(naphthyl)-N-phenylamino]biphenyl) was heated via application of electric current and vapor deposition was made onto the ITO transparent electrode at a deposition rate of 0.1 nm/second, whereby a hole injection layer having a thickness of 10 nm was produced.

Then, TCTA (tris(4-carbazoyl-9-ylphenyl)amine) was deposited on the hole injection layer at a deposition rate of 0.1 nm/sec to form a first hole transport layer having a thickness of 20 nm.

Further, H-233 was vapor-deposited on the first hole transport layer at a vapor deposition rate of 0.1 nm/sec to form a second hole transport layer having a thickness of 10 nm.

Then, the resistance heating boat containing a comparative compound 1 as a host compound and the resistance heating boat containing TBPe (2,5,8,11-tetra-tert-butylperylene) as a light emitting compound each were heated via application via application of electric current and vapor co-deposition was made onto the hole transport layer at a deposition rate of 0.1 nm/second, and 0.010 nm/second respectively, whereby a light emitting layer having a thickness of 20 nm was produced.

Then, H-232 was deposited on the light emitting layer at a deposition rate of 0.1 nm/sec to form a first electron transport layer having a thickness of 10 nm.

Further, TBPi (1,3,5-tris(1-phenyl-1H-benzimidazol-2-yl)benzene) was vapor deposited at a deposition rate of 0.1 nm/sec on the first electron transport layer. Thus, a second electron transport layer having a thickness of 30 nm was formed.

After forming a lithium fluoride layer having a thickness of 0.5 nm on the second electron transport layer, 100 nm thick aluminum was vapor deposited to form a cathode. Thus, an organic EL element 2-1 was prepared.

### (Preparation of Organic EL Elements 2-2 to 2-17)

Organic EL elements 2-2 to 2-17 were prepared in the same manner as preparation of the organic EL element 2-1 except that the comparative compound 1 used as a host compound in the formation of the light emitting layer was changed to the host compound as described in Table III.

### [Evaluation of Organic El Element]

### (Measurement of Relative Luminous Efficiency)

Each organic EL element thus produced was allowed to emit light by applying a constant electric current of 2.5 mA/cm² at room temperature (about 25° C.). The light emission luminance immediately after starting to emit light was measured with Spectroradiometer CS-2000 (produced by Konica Minolta, Inc.).

Then, the light emission luminance of the obtained organic EL element 2-1 was set to 100%, the relative light emission luminance of each organic EL element was determined, and this was regarded as the relative luminous efficiency. The results were indicated in Table III. The larger the value, the better the luminous efficiency.

TABLE III

| | | | Relative | |
|---|---|---|---|---|
| Organic | Light | | luminous | |
| EL | emitting | Host | efficiency | |
| element | compound | compound | (%) | Remarks |
| 2-1 | TBPe | Comparative | 100 | Comparative |
| | | compound 1 | | example |
| 2-2 | TBPe | Comparative | 78 | Comparative |
| | | compound 2 | | example |
| 2-3 | TBPe | Comparative | 92 | Comparative |
| | | compound 3 | | example |
| 2-4 | TBPe | Comparative | 74 | Comparative |
| | | compound 4 | | example |
| 2-5 | TBPe | Comparative | 70 | Comparative |
| | | compound 5 | | example |
| 2-6 | TBPe | Exemplified | 121 | Present invention |
| | | compound E-1 | | |
| 2-7 | TBPe | Exemplified | 162 | Present invention |
| | | compound E-6 | | |
| 2-8 | TBPe | Exemplified | 155 | Present invention |
| | | compound E-9 | | |
| 2-9 | TBPe | Exemplified | 150 | Present invention |
| | | compound E-15 | | |
| 2-10 | TBPe | Exemplified | 166 | Present invention |
| | | compound E-18 | | |
| 2-11 | TBPe | Exemplified | 168 | Present invention |
| | | compound E-25 | | |
| 2-12 | TBPe | Exemplified | 140 | Present invention |
| | | compound E-30 | | |
| 2-13 | TBPe | Exemplified | 153 | Present invention |
| | | compound E-33 | | |
| 2-14 | TBPe | Exemplified | 127 | Present invention |
| | | compound E-37 | | |
| 2-15 | TBPe | Exemplified | 133 | Present invention |
| | | compound E-77 | | |
| 2-16 | TBPe | Exemplified | 141 | Present invention |
| | | compound E-78 | | |
| 2-17 | TBPe | Exemplified | 155 | Present invention |
| | | compound E-90 | | |

As is clear from the results described in Table III, the organic EL element of the present invention using the compound of the present invention having a structure represented by Formula (1) as a host compound is excellent in luminous efficiency than the comparative example.

### Example 3

### [Preparation of Organic EL Element]

### (Preparation of Organic EL Element 3-1)

An ITO transparent electrode (anode) was prepared on a glass substrate of 50 mm×50 mm with a thickness of 0.7 mm by forming a film of ITO (indium tin oxide) with a thickness of 150 nm using a commercially available vacuum deposition apparatus, then by making patterning to it. The transparent substrate provided with the ITO transparent electrode was subjected to ultrasonic washing with isopropyl alcohol, followed by drying with desiccated nitrogen gas, and it was subjected to UV ozone washing for 5 minutes. Thereafter, the transparent substrate was fixed to a substrate holder of a commercial vacuum deposition apparatus.

The constituting materials for each layer were loaded in each resistance heating boat for vapor deposition in the vacuum deposition apparatus with an optimum amount. As a resistance heating boat for vapor deposition, a resistance heating boat made of molybdenum or tungsten was used.

After reducing the pressure of a vacuum tank to 4×10⁻⁴ Pa, the resistance heating boat containing HAT-CN (1,4,5,8,9,12-hexaazatriphenylene hexacarbonitrile) was heated via application of electric current and vapor deposition was made onto the ITO transparent electrode at a deposition rate of 0.1 nm/second, whereby a hole injection layer having a thickness of 10 nm was produced.

Then, α-NPD (4,4′-bis[N-(naphthyl)-N-phenylamino]biphenyl) was deposited onto the hole injection layer at a deposition rate of 0.1 nm/second, whereby a hole transport layer having a thickness of 40 nm was produced.

Then, H-232 as a host compound and a comparative compound 2 as a light emitting compound were co-deposited on the hole transport layer at a deposition rate of 0.1 nm/sec so as to have 94% and 6% by volume, respectively. A light emitting layer having a thickness of 30 nm was formed.

Thereafter, BCP (2,9-dimethyl-4,7-diphenyl-1,10-phenanthroline) was deposited on the light emitting layer at a deposition rate of 0.1 nm/sec to form an electron transport layer having a thickness of 30 nm.

After forming a lithium fluoride layer having a thickness of 0.5 nm on the electron transport layer, 100 nm thick aluminum was vapor deposited to form a cathode.

The non-light emitting surface side of the produced element was sealed by a glass case having a can shape under an ambience of high purity nitrogen gas having a purity of at least 99.999%. The electrode taken out wiring was set to obtain an organic EL element 3-1.

### (Preparation of Organic EL Elements 3-2 to 3-20)

Organic EL elements 3-2 to 3-20 were prepared in the same manner as preparation of the organic EL element 3-1 except that the type of the light emitting compound used for forming the light emitting layer and the presence or absence of the host compound were changed to the configuration described in Table IV.

### [Evaluation of Organic El Element]

### (Measurement of Relative Luminous Efficiency)

Each organic EL element thus produced was allowed to emit light by applying a constant electric current of 2.5 mA/cm² at room temperature (about 25° C.). The light emission luminance immediately after starting to emit light was measured with Spectroradiometer CS-2000 (produced by Konica Minolta, Inc.).

Then, the light emission luminance of the obtained organic EL element 3-1 was set to 100%, the relative light emission luminance of each organic EL element was determined, and this was regarded as the relative luminous efficiency. The results were indicated in Table IV. The larger the value, the better the luminous efficiency.

TABLE IV

| | | | Relative | |
|---|---|---|---|---|
| Organic | | | luminous | |
| EL | Light emitting | Host | efficiency | |
| element | compound | compound | (%) | Remarks |
| 3-1 | Comparative | H-232 | 100 | Comparative |
| | compound 2 | | | example |
| 3-2 | Comparative | — | 70 | Comparative |
| | compound 2 | | | example |
| 3-3 | Comparative | — | 78 | Comparative |
| | compound 4 | | | example |
| 3-4 | Comparative | — | 68 | Comparative |
| | compound 5 | | | example |
| 3-5 | Exemplified | — | 153 | Present |
| | compound E-1 | | | invention |
| 3-6 | Exemplified | H-232 | 148 | Present |
| | compound E-1 | | | invention |
| 3-7 | Exemplified | — | 146 | Present |
| | compound E-6 | | | invention |
| 3-8 | Exemplified | — | 140 | Present |
| | compound E-9 | | | invention |
| 3-9 | Exemplified | — | 142 | Present |
| | compound E-15 | | | invention |
| 3-10 | Exemplified | — | 149 | Present |
| | compound E-18 | | | invention |
| 3-11 | Exemplified | — | 150 | Present |
| | compound E-25 | | | invention |
| 3-12 | Exemplified | H-232 | 154 | Present |
| | compound E-25 | | | invention |
| 3-13 | Exemplified | — | 138 | Present |
| | compound E-30 | | | invention |
| 3-14 | Exemplified | — | 141 | Present |
| | compound E-33 | | | invention |
| 3-15 | Exemplified | — | 135 | Present |
| | compound E-37 | | | invention |
| 3-16 | Exemplified | H-232 | 145 | Present |
| | compound E-37 | | | invention |
| 3-17 | Exemplified | — | 142 | Present |
| | compound E-77 | | | invention |
| 3-18 | Exemplified | H-232 | 132 | Present |
| | compound E-77 | | | invention |
| 3-19 | Exemplified | — | 140 | Present |
| | compound E-78 | | | invention |
| 3-20 | Exemplified | — | 145 | Present |
| | compound E-90 | | | invention |

As is clear from the results described in Table IV, the organic EL element of the present invention using the compound of the present invention having a structure represented by Formula (1) as a light emitting compound is excellent in luminous efficiency than the comparative example.

### Example 4

### [Preparation of Organic EL Element]

### (Preparation of Organic EL Element 4-1)

An anode was prepared by making patterning to a glass substrate of 100 mm×100 mm×1.1 mm (NA45, produced by NH Techno Glass Corp.) on which ITO (indium tin oxide) was formed with a thickness of 100 mm. Thereafter, the above transparent support substrate provided with the ITO transparent electrode was subjected to ultrasonic washing with isopropyl alcohol, followed by drying with desiccated nitrogen gas, and it was subjected to UV ozone washing for 5 minutes.

On the transparent support substrate thus prepared was applied a 70% solution of poly (3,4-ethylenedioxythiphene)-polystyrene sulfonate (PEDOT/PSS, Baytron P AI4083, made by Bayer AG.) diluted with water by using a spin coating method at 3,000 rpm for 30 seconds to form a film, and then it was dried at 200° C. for one hour. Thus, a hole injection layer having a thickness of 20 nm was prepared.

The resulting transparent support substrate was fixed to a substrate holder of a commercial vacuum deposition apparatus. The constituting materials for each layer were loaded in each resistance heating boat for vapor deposition in the vacuum deposition apparatus with an optimum amount. As a resistance heating boat for vapor deposition, a resistance heating boat made of molybdenum or tungsten was used.

After reducing the pressure of a vacuum tank to 4×10⁻⁴ Pa, α-NPD was deposited onto the hole injection layer at a deposition rate of 0.1 nm/second, whereby it was produced a hole transport layer having a thickness of 40 nm.

Then, H-234 as a host compound and 2,5,8,11-tetra-t-butylperylene (TBPe) as a light emitting compound were co-deposited on the hole transport layer at a deposition rate of 0.1 nm/sec so as to have 97% and 3% by volume, respectively. A light emitting layer having a thickness of 30 nm was formed.

Thereafter, TPBi (1,3,5-tris (N-phenylbenzimidazol-2-yl) was deposited on the light emitting layer at a deposition rate of 0.1 nm/sec to form an electron transport layer having a thickness of 30 nm.

Further, after forming sodium fluoride with a film thickness of 1 nm on the electron transport layer, aluminum was vapor-deposited under the condition that the layer thickness becomes 100 nm to form a cathode.

The non-light emitting surface side of the produced element was sealed by a glass case having a can shape under an ambience of high purity nitrogen gas having a purity of at least 99.999%. The electrode taken out wiring was set to obtain an organic EL element 4-1.

### (Preparation of Organic EL Element 4-2)

An organic EL element 4-2 was prepared in the same manner as preparation of the organic EL element 4-1 except that the formation of the light emitting layer was changed. The light emitting layer was formed by using H-234 as a host compound, 2,5,8,11-Tetra-t-butylperylene (TBPe) as a light emitting compound, and a comparative compound 1 as a third component in such a way that the proportion of each component becomes 82%, 3%, and 15% by volume.

### (Preparation of Organic EL Elements 4-3 to 4-6)

Organic EL elements 4-3 to 4-6 were prepared in the same manner as preparation of the organic EL elements 4-2 except that the third compound was changed to a compound described in Table V.

### [Evaluation of Organic El Element]

### (Measurement of Relative Luminous Efficiency)

Each organic EL element thus produced was allowed to emit light by applying a constant electric current of 2.5 mA/cm² at room temperature (about 25° C.). The light emission luminance immediately after starting to emit light was measured with Spectroradiometer CS-2000 (produced by Konica Minolta, Inc.).

Then, the light emission luminance of the obtained organic EL element 4-1 was set to 100%, the relative light emission luminance of each organic EL element was determined, and this was regarded as the relative luminous efficiency. The results were indicated in Table V. The larger the value, the better the luminous efficiency.

TABLE V

| | | | | Relative | |
|---|---|---|---|---|---|
| Organic | Light | | | luminous | |
| EL | emitting | Host | Third | efficiency | |
| element | compound | compound | component | (%) | Remarks |
| 4-1 | TBPe | H-234 | — | 100 | Comparative |
| | | | | | example |
| 4-2 | TBPe | H-234 | Comparative | 110 | Comparative |
| | | | compound 2 | | example |
| 4-3 | TBPe | H-234 | Exemplified | 142 | Present |
| | | | compound | | invention |
| | | | E-6 | | |
| 4-4 | TBPe | H-234 | Exemplified | 151 | Present |
| | | | compound | | invention |
| | | | E-18 | | |
| 4-5 | TBPe | H-234 | Exemplified | 147 | Present |
| | | | compound | | invention |
| | | | E-37 | | |
| 4-6 | TBPe | H-234 | Exemplified | 138 | Present |
| | | | compound | | invention |
| | | | E-78 | | |

As is clear from the results described in Table V, the organic EL element of the present invention using the compound of the present invention having a structure represented by Formula (1) as a third component (assist-dopant) of the light emitting layer is excellent in luminous efficiency than the comparative example.

### INDUSTRIAL APPLICABILITY

The organic EL element material of the present invention is excellent in luminous efficiency, and an organic EL element to which the organic EL element material is applied may be used for the following apparatuses: display devices for television sets, personal computers, mobile devices, AV devices, teletext displays, and information in cars; and various lighting devices for home lighting, car interior lighting, backlights in watches and liquid crystals, billboard advertisements, traffic lights, light sources of optical storage media, light sources of light communication processors, and light sources of light sensors.

### DESCRIPTION OF SYMBOLS

1: Display

3: Pixel

5: Scanning line

6: Data line

7: Electric source line

10: Organic EL element

11: Switching transistor

12: Operating transistor

13: Capacitor

102: Glass cover

105: Cathode

106: Organic functional layer group

107: Glass substrate having a transparent electrode

108: Nitrogen gas

109: Water catching agent

200: Display device

300: Lighting device

A: Display section

B: Control section

C: Wiring section

F: Substrate

L: Emitting light

1. An organic electroluminescent element material comprising a compound having a structure represented by Formula (1), 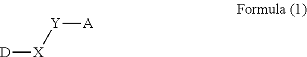
wherein D and A each represent a substituent; X and Y each represent a carbon atom, a nitrogen atom, an oxygen atom or a silicon atom which may have a hydrogen atom or a substituent; and at least one of X and Y is a carbon atom,
provided that in the substituent represented by D, when a structure in which a linking portion to a linker (X—Y) is replaced with a hydrogen atom is D-H; and in the substituent represented by A, when a structure in which a linking portion to the linker (X—Y) is replaced with a hydrogen atom is A-H, D-H has a higher energy level of a highest occupied molecular orbital (HOMO) than A-H, and A-H has a lower energy level of a lowest unoccupied molecular orbital (LUMO) than D-H,
wherein the substituent represented by D has a number of ring structures in the range of 3 to 15, and each of the ring structures may be bonded or condensed to each other; and
the structure represented by Formula (1) may further have one or more substituents, a plurality of the substituents may be bonded to each other to form a ring structure, and one saturated ring containing X and Y as ring member atoms may be formed.

2. The organic electroluminescent element material described in claim 1, wherein the ring structure represented by D in Formula (1) is a 5- or 6-membered aromatic hydrocarbon ring or heteroaromatic ring, and the compound contains three or more aforesaid ring structures.

3. The organic electroluminescent element material described in claim 1, wherein the substituent represented by A in Formula (1) has a ring structure, the ring structure is a 5- or 6-membered aromatic hydrocarbon ring or heteroaromatic ring, and the compound contains at least one aforesaid ring structure.

4. The organic electroluminescent element material described in claim 1, wherein the substituent represented by D in Formula (1) has one selected from the group consisting of a carbazole ring, an indolocarbazole ring, a diindolocarbazole ring, an acridan ring, and an indoloindole ring.

5. The organic electroluminescent element material described in claim 1, wherein the substituent represented by A in Formula (1) is at least one selected from the group consisting of a pyridine ring, a pyrimidine ring, a triazine ring, a dibenzofuran ring, an azadibenzofuran ring, a diazadibenzofuran ring, a carboline ring, a diazacarbazole ring, and a benzene ring containing at least one selected from a cyano group, a trifluoromethyl group and a halogen atom.

6. The organic electroluminescent element material described in claim 1, wherein the substituent represented by A in Formula (1) has two or more hetero atoms.

7. The organic electroluminescent element material described in claim 1, wherein X and Y in Formula (1) forms an ethylene linker.

8. The organic electroluminescent element material described in claim 1, wherein in Formula (1), the ring formed by bonding the substituents on X and Y to each other is a cyclohexyl ring, and the substituent represented by D and the substituent represented by A are each bonded to the cyclohexyl ring by syn addition.

9. The organic electroluminescent element material described in claim 1, wherein the electroluminescent element material is a light emitting material.

10. The organic electroluminescent element material described in claim 1, wherein the electroluminescent element material is a charge transport material.

11. The organic electroluminescent element material described in claim 1, wherein the compound having a structure represented by Formula (1) is a compound that forms an intramolecular or intermolecular exciplex.

12. An organic electroluminescent element having an anode, a cathode, and a light emitting layer between the anode and the cathode, wherein at least one of the light emitting layers contains the electroluminescent element material described in claim 1.

13. The organic electroluminescent element described in claim 12, wherein the light emitting layer further contains a host compound.

14. The organic electroluminescent element described in claim 12, wherein the light emitting layer further contains at least one of a fluorescence emitting compound and a phosphorescence emitting compound.

15. The organic electroluminescent element described in claim 12, wherein the light emitting layer further contains a host compound and at least one of a fluorescence emitting compound and a phosphorescence emitting compound.

16. A display device provided with the organic electroluminescent element described in claim 12.

17. A lighting device provided with the organic electroluminescent element described in claim 12.

18. A compound having a structure represented by Formula (1), 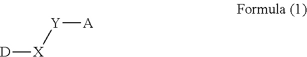
wherein D and A each represent a substituent; X and Y each represent a carbon atom, a nitrogen atom, an oxygen atom or a silicon atom which may have a hydrogen atom or a substituent; and at least one of X and Y is a carbon atom,
provided that in the substituent represented by D, when a structure in which a linking portion to a linker (X—Y) is replaced with a hydrogen atom is D-H; and in the substituent represented by A, when a structure in which a linking portion to the linker (X—Y) is replaced with a hydrogen atom is A-H, D-H has a higher energy level of a highest occupied molecular orbital (HOMO) than A-H, and A-H has a lower energy level of a lowest unoccupied molecular orbital (LUMO) than D-H,
wherein the substituent represented by D has a number of ring structures in the range of 3 to 15, and each of the ring structures may be bonded or condensed to each other; and
the structure represented by Formula (1) may further have one or more substituents, a plurality of the substituents may be bonded to each other to form a ring structure, and one saturated ring containing X and Y as ring member atoms may be formed.

19. The compound described in claim 18, wherein the ring structure resented by D in Formula (1) is a 5- or 6-membered aromatic hydrocarbon ring or heteroaromatic ring, and the compound contains three or more aforesaid ring structures.

20. The compound described in claim 18, wherein the substituent represented by A in Formula (1) has a ring structure having a 5- or 6-membered aromatic hydrocarbon ring or heteroaromatic ring, and the compound contains at least one aforesaid ring structure.

21. The compound described in claim 18, wherein the substituent represented by D in Formula (1) has one selected from the group consisting of a carbazole ring, an indolocarbazole ring, a diindolocarbazole ring, an acridan ring, and an indoloindole ring.

22. The compound described in claim 18, wherein the substituent represented by A in Formula (1) is at least one selected from the group consisting of a pyridine ring, a pyrimidine ring, a triazine ring, a dibenzofuran ring, an azadibenzofuran ring, a diazadibenzofuran ring, a carboline ring, a diazacarbazole ring, and a benzene ring containing at least one selected from a cyano group, a trifluoromethyl group and a halogen atom.

23. The compound described in claim 18, wherein the substituent represented by A in Formula (1) contains two or more hetero atoms.

24. The compound described in claim 18, wherein X and Y in Formula (1) forms an ethylene linker.

25. The compound described in claim 18, wherein in Formula (1), the ring formed by bonding the substituents on X and Y to each other is a cyclohexyl ring, and the substituent represented by D and the substituent represented by A are each bonded to the cyclohexyl ring by syn addition.
